(19) United States
(12) Patent Application Publication
(10) Pub. No.: US 2020/0000355 A1
(43) Pub. Date: Jan. 2, 2020

(54) ELECTRIC BIOPOTENTIAL SIGNAL MAPPING CALIBRATION, ESTIMATION, SOURCE SEPARATION, SOURCE LOCALIZATION, STIMULATION, AND NEUTRALIZATION.

(71) Applicant: Mohammad Mohammad Khair, Streamwood, IL (US)

(72) Inventor: Mohammad Mohammad Khair, Streamwood, IL (US)

(21) Appl. No.: 16/026,027

(22) Filed: Jul. 2, 2018

(51) Int. Cl. A61B 5/0408; A61B 5/04; A61B 5/044; A61B 5/00; A61B 5/0448; A61B 5/0478; A61B 5/0492; A61N 1/36; A61N 1/365

(52) U.S. Cl. CPC A61B 5/0408; A61B 2560/0223; A61B 5/044; A61B 5/0006; A61B 5/0448; A61B 5/0478; A61B 5/0492; A61B 5/04001; A61B 5/04087; A61B 5/6826; A61N 1/36103; A61B 5/746; A61B 5/0082; A61N 1/36507; A61N 1/36071; A61B 5/04012

(57) ABSTRACT

A leadless wireless ECG measurement system for measuring of bio-potentials includes at least one multi-contact bio-potential electrode assembly adapted for attachment to the patient's body to measure ECG. A processing unit is configured to produce a transfer function which computes estimated long-lead ECG signals based on the measured short-lead ECG signals from the plurality of contact points. This invention describes calibration process of short-lead ECG with standard lead ECG without requiring use of lead wires, only using a flexibly moving body part (finger) to calibrate patch. The invention describes use of algorithms for biopotential signal separation from mixed sources. The invention describes use of signal separation for identification of abnormal signals and the localization of biopotential source tissue. This invention describes effective biopotential evoked potential stimulation. The invention also describes biopotential neutralization of undesired biopotential.

### BACKGROUND OF THE INVENTION

### 1. Technical Field

This invention relates generally to electrophysiologic bio-potential signals measurement, source separation, localization, mapping, calibration and estimation, neutralization, and stimulation devices and methods. Said bio-potential signals include cardiac electrocardiographs (ECG) and cardiac muscle electrograms (EGM), fetal ECG, the brain electroencephalographs (EEG), neural activity, and Muscle electromyograms (EMG), evoked biopotentials and pacing signals, and artifacts signals.

In one particular aspect, it relates to a new and improved leadless wireless ECG measurement system and method for measuring of bio-potential electrical activity of the heart that uses measurements obtained across a much smaller separation distance between electrode contact points, called short leads, but yet retains the presentation of ECG waveforms which corresponds closely to existing ECG measurement across standard electrode contact locations, thereby preserving the biopotential signal waveform morphology, amplitude, and frequency components. A system and method for calibration and transformation of the short leads (i.e. source leads) waveforms to long leads (i.e standard leads, or target leads) waveforms is described. For the purpose of this invention, the term long-lead is defined as a biopotential signal measured across at least two electrode contacts located on the body tissue that represent at least one of: a target standard lead, a target non-standard lead, a lead with longer spacing between contacts, a lead to be estimated, or an output signal from a model. Whereas the term short-lead is defined as a biopotential signal measured across at least two electrode contacts located on the body tissue that represent at least one of: a source standard lead, a source non-standard lead, a lead with shorter spacing between contacts, a lead to be measured, an input signal into a model. Short leads and long leads can be measured either invasively or noninvasively. In some embodiments, a long-lead that is estimated as an output of a model can also be subsequently reused as an input to another model similar to a short lead.

Also described are systems and methods for source separation of desired biopotential signal from mixed desired and undesired biopotential signals, such as separating fetal ECG from mixed fetal and maternal ECG, or separating EEG from mixed EMG and EEG, which may be overlapping in the frequency spectral content.

Also we describe systems and methods for stimulation of evoked biopotential or cardiac pacing (using stimulation with an externally provided electric current) to affect a target tissue site with a desired particular signal waveform morphology and timing so as to generate a desired evoked potential and its effect. Such methods advantageously enable effective stimulation yet using remote noninvasive application of stimulation devices reducing the risk of surgically implanted devices to the patient, and reducing cost as well.

Also we describe systems and methods for localization (mapping) of sources of undesired biopotential signal physiologic sources (such as cardiac arrhythmia or fibrillation, or brain seizures or tremors).

Finally, we describe systems and methods for temporary neutralization of undesired biopotentials including those biopotentials causing cardiac arrhythmia or fibrillation.

Related U.S. Pat. No. 8,838,218, and divisional patent application US20160022161A1 are incorporated herein in their entirety. Additionally this non-provisional patent application is based on provisional patent application 62/529,079.

### 2. Prior Art

As is generally known in the art, an electrocardiograph is widely used by the medical profession in order to obtain an electrocardiogram (ECG) which is a measurement of bio-potential electrical activity of the heart from the surface of the skin. The conventional 12-lead electrocardiograph typically requires at least 10 wires to be attached via electrodes to the body of the patient at one end and to the electrocardiograph at the other end so as to measure the bio-potentials representing heart-signals and to transfer them via bipolar and unipolar leads into a 12-lead electrocardiogram.

ECG measurements have been conducted for over 200 years, and a standard configuration of the measurement vector leads have been adopted by the medical and engineering communities. This standard of leads formation and configuration require substantial separation of points of measurements on the surface of the skin, which necessitates connection of two remote points by lead wires into an instrumentation amplifier. This large separation between electrode contact points maximizes the surface area of the skin between the measurement electrode points and therefore maximizes the impedance, and measured voltage potential across the contact electrodes.

In early years, this was necessary for measurement of ECG due to lack of electronics that satisfy the measurement quality, signal-to-noise ratio, and cost constraints. Today, however, current electronics resolution, noise rejection, and amplification strength allows for ECG measurements across a much smaller separation distance between the contacting electrodes. ECG measurement standards, however, have been largely set and adopted with the original configuration of contact points preserving large separation distances between contacting electrodes.

The instrumentation amplifier is ideally used for the measurement of the ECG. The instrumentation amplifier typically rejects common mode noise using a common reference electrode to its two bipolar inputs, and amplifies the difference in potential between the two measurement electrodes as the measured bio-potential value. This bio-potential changes dynamically with the cardiac contraction and dilation due to depolarization and re-polarization of the cardiac muscle. The electric activity emanates from the Sinoatrial node (SA node) and spreads through the Purkinji fibers from the Atrial upper portion to the ventricular portion of the cardiac muscle.

The electric activity surfaces from the cardiac muscle to the skin and dissipates throughout the conductive skin layer. Since the skin has electric impedances, the conductivity of the electric current varies depending on the direction of the measurement and the separation distance of between the measurement electrodes. The skin impedance varies dynamically depending on multiple factors, including the hydration status of the skin, blood flow vasodilators or vasoconstrictors, medications, cardiac output to name a few.

It will be noted that the 12-lead ECG provides spatial information about the heart's electrical activity in 3 approximately orthogonal directions. The orthogonal directions are namely (1) Right to Left, (2) Superior to Inferior, and (3) Anterior to Posterior. Thus, the standard ECG measurement involves the attachment of six electrodes to the chest or precordial area of the patient to obtain recordings of leads V1 through V6 and the attachment of four electrodes to the arms and legs in order to obtain recordings of leads I, II, III, AVR, AVL, and AVF. Subsequent to the attachment of the ten electrodes to the patient, there is then required the connecting of ten specific wires between each particular electrocardiograph terminal and the related electrodes of predetermined position.

One of the disadvantages encountered in the operation of the conventional ECG devices is that they utilize a large separation spacing between the electrode contact points which requires maximum surface area of the skin and thus maximizes impedance and measured voltage potential across the contact electrodes. Another disadvantage suffered by the prior art ECG devices is that the numerous lengthy terminal wires coupled to the electrodes will frequently obstruct the patient and limit the freedom of movement of the patient. Further, the terminal wires often become intertangled with one another during their use, thereby rendering them difficult and cumbersome for the physician and/or technician. In addition, the conventional ECG devices and their attaching electrodes suffer from the problem of having a relatively large footprint.

In U.S. Pat. No. 6,441,747 to Khair et al. and U.S. Pat. No. 6,496,705 to Ng et al., there are disclosed a wireless, programmable system for bio-potential signal acquisition which includes a base unit and a plurality of individual wireless, remotely programmable transceivers connected to patch electrodes. The base unit manages the transceivers by issuing registration, configuration, data acquisition, and transmission commands using wireless techniques. The bio-potential signals from the wireless transceivers are demultiplexed and supplied via a standard interface to a conventional ECG monitor for display. Further, there is shown in U.S. Pat. No. 7,403,808 to Istvan et al. a cardiac monitoring system for detecting electrical signals from a patient's heart and wirelessly transmit the signals digitally to a remote base station via telemetry. The base station converts the digital signals to analog signals which can be read by an ECG monitor. In U.S. Pat. No. 5,862,803 to Besson et al., there is described a wireless medical diagnosis and monitoring equipment which includes an evaluation station and a plurality of electrodes which are arranged on a patient. Each of the plurality of electrodes includes elementary sensors, sensor control, transceivers, and transmission control units which are integrated in one single semiconductor chip. In U.S. Pat. No. 4,981,141 to Segalowitz, there is disclosed an electrocardiographic monitoring system in which the heart-signal sensing electrodes are each coupled to the heart-signal monitor/recorder by respective wireless transmitters and corresponding respective receiving wireless receivers in a base unit.

Therefore, it would be desirable to provide a leadless wireless ECG measurement system and method for measuring of bio-potential electrical activity of the heart which operates on a more efficient and effective basis. Further, it would be expedient that the ECG measurement system overcomes all of the afore-mentioned shortcomings of the prior art discussed in connection with the application of the conventional electro-cardiographs used to obtain the electrocardiogram. The present invention represents a significant improvement over the aforementioned prior art U.S. Pat. Nos. 6,441,747; 6,496,705; 7,403,808; 5,862,803; and 4,981,141 which are hereby incorporated by reference in their entirety.

### BRIEF SUMMARY OF THE INVENTION

In one aspect of this invention, a wireless ECG measurement system of the present invention performs measurements of ECG in a leadless configuration using a minimal number of bio-potential measurements across a short distance (approximately 1 to 3 inches and referred to as “short-leads”) which represent input waveforms to a model, and then maps these ECG measurements using a plurality of identified mathematical models (or functions) to the standard 12-lead configuration representing the model outputs. The final output is presented to the end user as an estimated calculated ECG of up to 12 standard ECG leads. The short lead measurements do not require extended lead wires due to the proximity of distance between the measurement points, and the measurement electrode contact points can be integrated in a single electrode patch that has a plurality of contact points with the surface of the skin for conducting the measurements.

In view of the foregoing background, it is therefore an object of the present invention to provide a leadless wireless ECG measurement system and method for measuring of bio-potential electrical activity of the heart of improved design and performance. It is another object of the present invention to provide a leadless wireless ECG measurement system and method for measuring of bio-potential electrical activity of the heart which uses measurements across a much smaller separation distance between the electrode contact points. It is still another object of the present invention to provide an ECG measurement system and method which is much more compact in its form and coverage area between the contacting points on the surface of the skin. It is still yet another object of the present invention to provide an ECG measurement system and method which produces a higher degree of comfort for the patient by eliminating lead wires extending to distal electrodes, is easier to use and has more flexibility of placement for the clinician without a tradeoff of accuracy, and has a smaller footprint than the conventional ECG devices. Another object of this invention is to provide novel calibration methods between short lead ECG signals and long lead ECG signals (or their components) with improved usability and human factors that promote ease of use and reduced user error in application of the device, especially during remote or mobile ECG monitoring when a clinician is not available.

The term patient in this document refers to non-healthy or healthy subjects using the systems or methods described herein.

These and other objects, features and advantages of the invention are provided by a leadless wireless ECG measurement system for measuring of bio-potential electrical activity of the heart in a patient's body which includes at least one multi-contact bio-potential electrode assembly adapted for contact to the patient's body.

In one embodiment, the electrode assembly is formed of an electronic patch layer and a disposable electrode layer. The disposable electrode layer has a plurality of contact points for engagement with the surface of the patient's body and is configured to measure short-lead ECG signals in response to electrical activity in the heart. An adhesive layer on the disposable electrode functions for attachment of the electrode layer and the patch to the patient's body.

In a second embodiment, the electrode layer for the ECG sensing device is a dry electrode that is in contact with the patient's skin or tissue. The dry electrode can be in the form of conductive contact point or contact surface or contact line segment or wire that when in touch with the skin or tissue conducts the ECG electric current generated by the heart for measurement of cardiac electric bio-potential. In a preferred embodiment, the dry electrode are adapted to a body part, including a finger, arm, wrist, hand, or neck, but preferably a flexibly moving finger or wrist, whereas the attached ECG sensing device including a patch, wrist watch, a bracelet, a ring, or a band is in touch or contact with the skin and providing a reference contact point for measuring bio-potential.

Calibration: The ECG sensing device measuring the electric bio-potential between the reference contact on the skin or tissue, and at least a second relative contact on the body, whereas this relative contact is preferably representing a contact point of the defined standard ECG leads points on the body. In an embodiment, the reference contact electrode is on the interior surface of the ECG sensing device while the relative contact electrode is on the exterior of the ECG sensing device, or its extension. The process can iterate to acquire multiple bio-potentials between the same reference contact and a plurality of relative contact points, in each step measuring the bio-potentials as first component and second components defining a standard lead. This can be accomplished with an ECG biopotential measurement or sensing device attached to a flexibly moving body part, such as a finger, a hand, or an arm, and take the form of a device, preferably mobile device that is connected or tethered to a body attached sensor, or a wearable sensory device, including a wrist watch, a bracelet, a ring, a band, or a necklace. The ECG biopotential measurement or sensing device comprises of an electrical components for sensing and digitization of the biopotential, telecommunication electronics for wireless communication of the data, a computational processing unit and memory electrical components, and includes at least two electrodes that are either integrated or in extended attachment with the device. The electrodes can be disposable but are preferably dry electrodes that are reusable. In one embodiment, the calibration probe has one electrode on its exterior surface and another on its interior surface that both act as conductive dry electrodes used for measurement of the biopotential and interface to the patient skin or tissue. The ECG measurement device can be adapted with an extension (tethered) sensory calibration probe comprised of patient's skin contacting electrodes, preferably dry electrodes, that are connected to the device with conductive wires. In a preferred embodiment, the calibration probe is comprised of a finger covering calibration probe that has an electrode that is conductive on the exterior surface and conductive on the interior surface, with an insulating layer in between the two said surfaces. The sensing device measuring differential electric biopotential between the two conductive surfaces and can be attached to said sensing conductive surfaces or tethered with a cable containing two conductive wires coupled to each of the said conductive surfaces. The sensing device is adapted for obtaining ECG measurements from the flexibly moving body part, preferably a finger, but also including a wrist, or an arm, or a hand.

The ECG measurement device in connection with the body, wrist, arm, hand, or finger and using either tethered electrodes or integrated electrodes for sensing electric biopotential from the surface of the skin is used to obtain an ECG lead between the two electrodes contact points to the skin, one contact on the flexibly moving body part and the other on the chest, using the interior conductive surface of the sensing calibration probe, and a second contact point on another body skin area using the exterior conductive surface of the sensing calibration probe, said second contact point being preferably defined as part of the standard contacts locations for measuring the 12 lead configuration ECG. This differential biopotential signal between said first and second contacts is then used, for calibration purposes, as a long ECG lead signal, that represents an output signal being estimated by the transfer function, or model, while using a short ECG lead as an input signal to said model. The short ECG lead signal is measured across a small spacing from the wearable ECG patch. The transfer function is adaptively identified using any of the herein mentioned nonlinear or linear system identification methods, preferably state-space linear models.

The ECG sensing calibration probe is a device comprised of electrodes and attached to a finger (ring or finger probe), or a hand (band), or a wrist (watch or bracelet or band), or an arm (band) using a connected measuring device (either directly attached or using a tethered extension cable). The ECG sensing calibration probe is used in touching the plurality of contact points on the body forming the desired standard leads. In one embodiment, the calibration probe device is adapted for engagement with any of the five fingers, on either the right or left hand, including the thumb or index or middle finger or ring or pinky fingers. The flexibly moving body part is brought in contact, for a brief calibration time duration, with a plurality of patient's skin contact points, using biopotential electrodes, preferably dry electrodes, to measure the plurality of calibration long-leads of ECG biopotentials between each of these contact points on the body and a common reference contact point on the moving body part (finger or hand or arm). During the calibration (mapping) process, each bio-potential voltage can be measured between a reference contact (on the flexibly moving body part) and the relative contact points on the body forming a biopotential calibration lead(s) ECG signal over the iteration of the process. During each step, a calibration mapping transfer function is generated between the target calibration lead (long lead) and the short lead(s) measured across a smaller electrode spacing using a wearable patch ECG sensing device.

The calibration process involves repetitive measurement steps of different calibration leads, each representing the biopotential measured between the ECG measurement sensing device at a first reference contact on the body, including the wrist, or arm, or hand, or fingers, and the second relative contact at the standard ECG contact point on the body. A plurality of a pair of calibration leads are selected and are either added or subtracted (based on their polarity) to obtain the desired standard ECG long leads in order to create the desired standard ECG leads sets and to present their signals for clinical diagnosis.

The biopotential is the voltage difference between the electrode on the calibration probe's (ECG sensing device) exterior conductive surface (preferably dry electrode) or its extension and the interior conductive surface (preferably dry electrode). This electric bio-potential will be measured to form a first component of a standard lead measurement. This process is repeated for measuring an electric bio-potential between the same reference contact and a second relative contact forming a second component of a standard lead measurement, whereas the first relative contact and the second relative contact represent the two contacts of the standard lead (for any of the bipolar or unipolar standard leads). The subtraction of the first component of the standard lead from the second component of the standard lead (or vice versa based on polarity) will result in the desired standard lead itself. The process is repeated for the rest of the standard leads and their components using the contact points defining the standard leads and the common reference point on the flexibly moving body part. This calibration process advantageously uses the flexibly moving body part as a conductive layer (using electric current conduction through the skin) for measuring biopotential across two contact points on the body, defined by their shared common reference point contact on the moving body part itself. This advantageously eliminates the use of the wires or lead set during the calibration process, and reduces confusion with the multiple contact electrodes, and wire colors, and eliminates the time consumed in sorting entangled wires or disinfecting them, and associated errors as a result of confusing leadsets. This novel calibration process uses a common reference based on the moving body part (finger) through a series of touch points on the body's standard ECG lead contact points, without any lead wires, to advantageously yield an equivalent result to a calibration process that uses traditional, more cumbersome, lead wires. The result is an easier, more manageable, calibration process that the patient can much more easily self-calibrate their wearable patch sensors at home, preferably with the guidance of graphical display training software, without the assistance of professional clinicians.

In a first step, the calibrating device measures the short leads as input signals and measures a first component of the standard lead as output signals and identifies a first model using linear or non-linear system identification methods, preferably linear state-space models. In a second step, the calibrating device measures the short leads as input signals and measures a second component of the standard lead as output signals and identifies a second model using linear or non-linear system identification methods, preferably linear state-space models. This completes the calibration process for the target standard lead that can be calculated as completing a Kirchhoff's voltage loop between the first and second components measured during the calibration step. In a subsequent monitoring step, the system measures the short lead ECG signals only used as input signals to the previously identified (during calibration) first model to estimate said first component of the target standard lead, and also applies the same short lead signals as input signals to the previously identified (during calibration) second model to estimate said second component of the target standard lead. The standard lead itself is then estimated using the estimated first and second components by completing the Kirchhoff's voltage loop by either adding or subtracting the said estimated first component and said second component of the standard lead (per polarity configuration of the leads) to result in the estimated target standard lead. Note that the subtraction of two estimated unipolar leads simultaneously acquired or estimated results in a bipolar acquired or estimated lead.

Advantageously, the electronic layer of the patch or the body part attached device in the present invention further is coupled to or includes a transceiver unit for transmitting and receiving wireless communications with a base station or with other patch electrode assemblies.

Further, a processing unit is provided and is configured to produce a transfer function which computes as output signals the standard long-lead ECG signals, or their first and second components, based on the input signals of measured short-lead ECG signals from the plurality of contact points. The transfer functions are identified adaptively using any of the linear or non-linear system identification methods referenced herein, preferably linear state-space models.

The calibration measurement device or electrode calibration probe is preferably attached to a flexibly moving body part and is in either wired, or preferably wireless, communications of the sensed ECG signals, in either analog or digital form, to another device that is measuring the short lead ECG signals, including an ECG patch. In another embodiment, both the calibration device attached to a flexibly moving body part is in either wired communications, or more preferably wireless communications, of the sensed ECG signals (in either analog or digital form) to a receiving base station that is shared in communication with the short lead ECG sensing device, including an ECG patch. The base station includes a computational processor, memory, antenna, and a wireless transceiver for transmitting and receiving communications with the plurality of contact points in the electrode layer. The wireless communications received by the wireless transceiver includes the standard long-lead ECG signals, and/or its plurality of components as defined by biopotential between the reference contact point and the standard contact points sensed by the calibration device, as well as the short lead ECG signals from the patch contact points.

In addition, a display monitor is coupled to receive the long-lead ECG signals from the base station for displaying meaningful information.

Also key disadvantage of current implantable devices for stimulation of cardiac pacing or neuromodulation (such as vagus nerve neuromodulation) is that they require surgical procedures to implant and are invasively inserting electric current to target tissue. Thus introducing significant risk to the patient, and require surgical maintenance or service procedures.

Furthermore, current procedures to neutralize a source of cardiac arrhythmia or tissue depicting abnormal electrical activity (such as brain seizure activity) are limited to ablation or surgical removal of tissue which is very invasive results in permanent tissue trauma. Drug therapies reduce but do not cure the abnormal electrical conduction issues with the tissue.

These and other features and advantages of the disclosed system and methods reside in the construction of parts and the combination thereof, the mode of operation and use, as will become more apparent from the following description, reference being made to the accompanying drawings that form a part of this specification wherein like reference characters designate corresponding parts in the several views. The embodiments and features thereof are described and illustrated in conjunction with systems, tools and methods which are meant to exemplify and to illustrate, not being limiting in scope.

### BRIEF DESCRIPTION OF THE SEVERAL VIEWS OF THE DRAWING

FIG. 1 is a schematic representation of a wireless ECG measurement system for use with a patient so as to acquire ECG signals via “short leads” and to deliver them to an ECG monitor, constructed in accordance with the principles of the present invention;

FIGS. 1(a) through 1(f) depict the various preferred locations that a plurality of electrode assemblies of the present invention may be placed on a patient's body;

FIG. 1(g) depicts the electrodes 16a-16d and the electrodes 18a-18f disposed on the patient's body to obtain the respective limb lead signals and precordial lead signals in the standard ECG measurement system;

FIG. 2 is an enlarged, perspective view of the multi-contact bio-potential electrode assembly of FIG. 1, with the electronic patch layer being illustrated separate and apart from the disposable electrode layer;

FIGS. 2(a) through 2(g) depict various configurations of the disposable electrode layer each having a plurality of contact points arranged on its surface;

FIG. 3 is a block diagram of the electronic patch layer of FIGS. 1 and 2;

FIG. 4 is a block diagram of the base station of FIG. 1;

FIG. 5 is a system transfer function which illustrates the time domain and frequency domain relationships between the input and output time series;

FIG. 6 is flow diagram of an exemplary embodiment of a calibration procedure;

FIG. 7 is flow diagram of an exemplary embodiment of an alternate calibration procedure; and

FIG. 8 is flow diagram of an exemplary embodiment of a re-calibration procedure;

FIGS. 9(a), 9(b), 9(c), and 9(d) depict various configurations of locations that the electrode assembly may be placed on the head's scalp surface of a patient;

FIG. 10 is a flow diagram of an exemplary embodiment of a physiologic parameter monitoring procedure; and

FIGS. 11(a), 11(b), 11(c), and 11(d) are sample plots depicting estimated long standard leads I, II, and III from measured short leads at locations RA, LA, and LL.

FIG. 12a depicts wrist watch or bracelet use for calibration of two components of a long leads of a target standard lead with the patch short leads. Kirchoff's voltage loop components are measured between finger point interior of calibration probe and its exterior surface touching a contact point of a standard ECG lead. When two such components are measured for two contact points forming a standard lead, then the standard lead is computed by subtracting the two measured long lead components.

FIG. 12b depicts a Patch and Watch attached to patient.

FIG. 13 depicts interior contact conductor surface in touch with the skin, covered by an insulator, and then an exterior contact conductor surface of a wrist watch or bracelet device.

FIG. 14 depicts interior contact and exterior contact of a wrist watch or bracelet device with an extension lead wire to a finger.

FIG. 15 depicts a watch device displaying the ECG waveform that is either acquired on the device or computed estimate of an ECG waveform or received by wired or wireless transmission from another device.

FIG. 16 depicts the ECG patch.

FIG. 17 depicts the ECG patch attachment on a patient's body.

FIG. 18 depicts the ECG patch with an exterior dry contact electrode for touching by either the left or right hand to form a biopotential measurement with the interior electrode layer at the bottom of the patch attached to the patient's skin and sensing the short lead(s).

FIG. 19 depicts the patch interior electrode layer at the bottom of the patch attached to the patient's skin and sensing the short lead(s).

FIG. 20 depicts fetal ECG detection with two patches. We acquire from a first site primarily maternal ECG, and from a second abdominal site mixed maternal and fetal ECG waveforms.

FIG. 21 depicts first configuration for fetal ECG detection with a single patch and extension electrodes. We acquire from a first site primarily maternal ECG, and from a second abdominal site mixed maternal and fetal ECG waveforms.

FIG. 22(a) depicts second configuration for fetal ECG detection with a single patch and extension electrodes. We acquire from a first site primarily maternal ECG, and from a second abdominal site mixed maternal and fetal ECG waveforms.

FIG. 22(b) depicts a configuration for fetal ECG detection for multiple fetuses, such as twins, with a plurality of abdominal patches for mixed maternal-fetal ECG, and a single maternal-only patch.

FIG. 23 depicts fetal ECG separation (extraction) from mixed maternal and fetal ECG waveforms.

FIG. 24 depicts the surface ECG estimation using pacemaker acquired electrogram.

FIG. 25 depicts the cardiovascular system with its circulatory system of pulmonary and systemic circulations and electrogram measuring calibration probe and electrodes extending out of the calibration probe in a cardiac chamber.

FIG. 26 depicts the leadless ECG system including a finger calibration probe sensor tethered to a mobile device via a communicating interface unit that is used for acquiring the long lead ECG signals. The mobile device is in simultaneous wired or wireless communication of the long lead signals as well as a wireless communication of wearable ECG sensor patch used to obtain short lead ECG signals. This configuration enables the system identification of a system transfer function for calibration mapping and estimation of output long leads ECG output signals using the input short leads ECG signals. The mobile device display is used to guide the user through the calibration process of all desired standard or long ECG leads. One of the wearable sensor patch, or the mobile device, or the interface unit can be configured for computation of the calibration function and/or estimation of output ECG leads and other useful information. The mobile device can also be used for long range communication of acquired ECG signals or estimated output long lead ECG signals or related useful information on patient health.

FIG. 27 depicts the leadless ECG system including a finger calibration probe sensor tethered to a wearable sensor patch via a communicating interface unit that is used for acquiring the long lead ECG signals. The finger calibration probe is in simultaneous communication of the long lead ECG signals with wearable sensor patch which used to obtain short lead ECG signals, which enables the system identification of a system transfer function for calibration mapping and estimation of output long leads ECG output signals using the input short leads ECG signals. The mobile device display is used to guide the user through the calibration process of all desired standard or long ECG leads. Either the wearable sensor patch or the mobile device can be configured for computation of calibration function and/or estimation of ECG leads and other useful information. The mobile device can also be used for long range communication of acquired ECG signals or estimated output long lead ECG signals or related useful information on patient health.

FIG. 28 depicts the leadless ECG system including a finger calibration probe sensor that is used for acquiring and wireless communicating of the long lead ECG signals. The wireless calibration probe is in simultaneous wireless communication of the long lead signals with a wireless wearable ECG sensor patch used to acquire and communicate short lead ECG signals, which enables the system identification of a system transfer function for calibration mapping and estimation of output long leads ECG output signals using the input short leads ECG signals. The mobile device display is used to guide the user through the calibration process of all desired standard or long ECG leads. The mobile device can also be configured for communicating with the wireless finger calibration probe and/or the wearable sensor patch for computation and estimation of ECG leads and other useful information. The mobile device can also be used for long range communication of acquired ECG signals or estimated ECG signals or related useful information on patient health. In an alternative embodiment, the computation of transfer function and/or output long lead ECG signal estimates can take place in the wearable sensor patch itself.

FIG. 29 depicts the leadless ECG system finger calibration probe sensor used to obtain long lead signals for calibration transfer function mapping and estimation of short leads ECG signals acquired by the patch or wearable sensor device.

FIG. 30 depicts the calibration steps of component leads forming a Kirchoff's voltage loop with a standard ECG lead.

FIGS. 31(a), 31(b), and 31(c) depict example placement, and process steps flow chart, respectively, for neutralization of undesired biopotential at a target tissue site using an access tissue site stimulation of biopotential.

FIGS. 32(a), 32(b), and 32(c) depict example placement, and process steps flow chart, respectively, for stimulation of desired biopotential at a target tissue site using an access tissue site stimulation of biopotential.

FIGS. 33(a), 33(b), 33(c), and 33(d) depict example placement, and process steps flow chart, respectively, for completing a neural conduction connection between a source neural activity 3321 that has its neural conduction pathway 3327 disconnected from its target neural destination 3322. This is accomplished by measurement of a source biopotential 3323, for example a neural biopotential of the spinal nerve or similarly any other neural pathway(s) or muscular tissue, or its estimation using a remote access tissue site 3324 (using two contact electrodes, for example on the skin) and a first monitoring transfer function model, preferably defined using system identification and more preferably using linear state space models, then determination or estimation of the biopotential waveform at the source neural pathway 3322, then communication of this estimated source neural biopotential waveform to a second stimulation access tissue site 3326, whereby an estimation is made using a second model transfer function, preferably defined using system identification and more preferably using linear state space models, used to estimate required input waveform to be injected at a stimulation access tissue site 3326 (using two contact electrodes) to result in an evoked biopotential stimulus of desired waveform at the target tissue site 3325 of neural pathway for continuation of neural activity. The transfer functions are preferably identified using linear or nonlinear system identification methods, and more preferably using a linear state space system model representation. In an embodiment, both the measurement access tissue site 3324 and the stimulation access tissue site 3326 are both the same electrode contact points, however, they can also be different sites and electrode contact points, depending on the clinical needs of the patient.

### DETAILED DESCRIPTION OF THE INVENTION

In one aspect of this invention, the system and methods provides means of noninvasive effective stimulation of biopotential electrical activity to a remote tissue by controlling the timing and morphology of the waveform to maximize effective desired stimulation signal and induced behavior of muscular or nerve or tissue biopotential stimulation.

In another aspect of the invention, the system and methods provide for inducing a noninvasive effective reverse stimulation of biopotential electrical activity to neutralize an abnormal tissue electrical activity to reduce or eliminate its impact on the muscle, nerve, or tissue. This is accomplished by coupling the modeling of electrical tissue characteristics between point of insertion of stimulus signal and point of effective control signal with an adaptive control system to continuously adapt and vary the stimulus signal to yield optimum effective neutralization and control of the undesired signal at the target tissue of interest. The modeling of tissue characteristics can be applied bi-directionally between target tissue and stimulus tissue locations. In one diagnostic step the biopotentials at the target are used as inputs into the model and the monitored biopotentials at the stimulus tissue area are used as model outputs. This enables the remote monitoring of the biopotential activity within the target tissue. In another stimulation step the reverse is done, whereby the inserted stimulus biopotential activity at the stimulus tissue is used as model inputs and the evoked biopotential at the target tissue are used as model outputs. The diagnostic and stimulation models represent transfer functions depicting the relationships between input biopotentials and output biopotentials and are best identified using linear and/or nonlinear system identification methods, and preferably using linear state space models. The diagnostic step is used to first evaluate the abnormal biopotential at the source target tissue and in a second step determine effective desired neutralization pattern at the source target tissue that is needed to effectively neutralize need to be inserted during the stimulation step to effectively neutralize abnormal activity at that target tissue. An adaptive control system can include model-based predictive adaptive control, known in the art, or model-free adaptive control, such as described by patent U.S. Pat. No. 6,055,524 incorporated herein by reference.

In one embodiment, an electrode assembly is formed of an electronic patch layer and a disposable electrode layer. The disposable electrode layer has a plurality of contact points for engagement with the surface of the patient's body and is configured to either measure short-lead biopotential signals in response to electrical activity in the body and/or to stimulate a desired biopotential activity in the body at a target location(s), and/or to neutralize an undesired biopotential activity in the body at a target location(s). An adhesive layer on the disposable electrode functions for attachment of the electrode layer and the patch to the patient's body.

In yet another aspect of the current invention, neural stimulation is affected to function as an electronic means to bridge a nonfunctional or cut conduction pathway between two ends of a nerve, effectively rendering the nerve conduction pathway functional again. The neural pathway is bridged by two steps, the first a measurement between the source nerve signaling and a monitoring access point, followed by wired or wireless communication between the monitoring access point and a stimulation access point, which then applies the biopotential signal to stimulate an effective pacing current at the target nerve, thus completing the conduction pathway electronically across the two nerve endings.

In another aspect, the present invention shown in the drawings and described in detail in association with a leadless wireless ECG system for measuring of bio-potential electrical activity of the heart is not intended to serve as a limitation upon the scope or teachings thereof, but is to be considered merely for the purpose of convenience of illustration of one example of its application. The same system structure and methods are applicable to other electrophysiologic signals including brain EEG waveforms, muscular EMG waveforms, neural biopotential activity, ocular muscle waveforms, and respiratory biopotential waveforms.

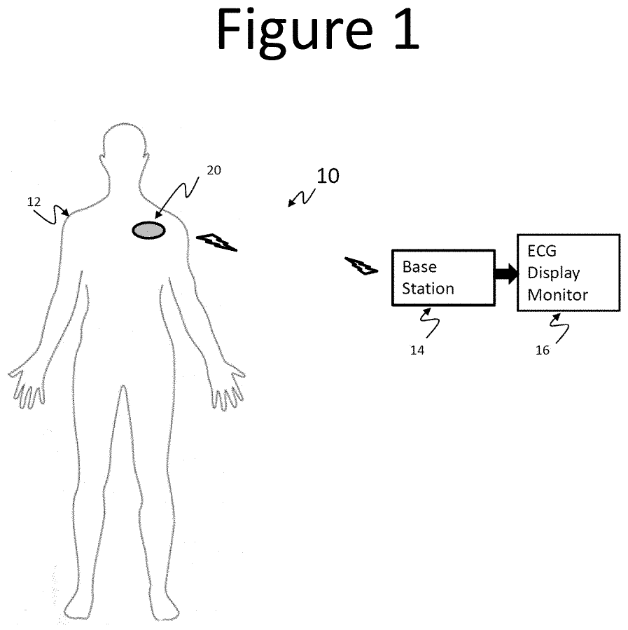

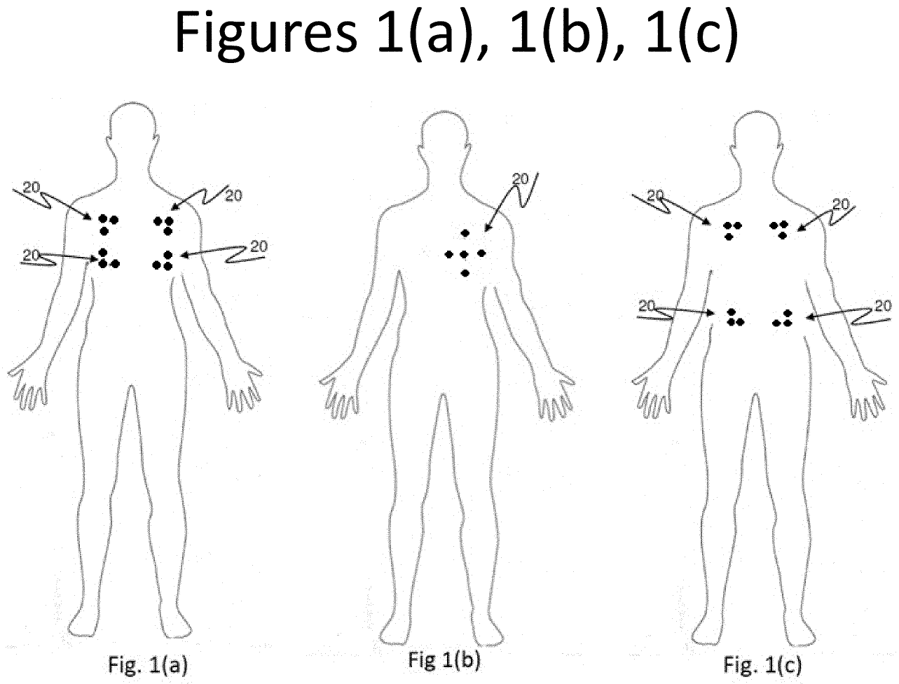

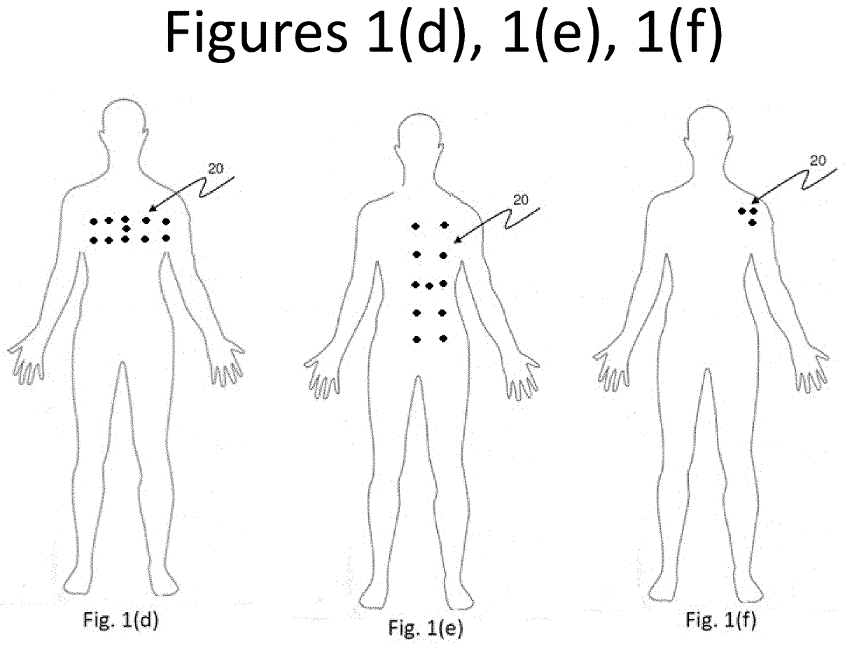

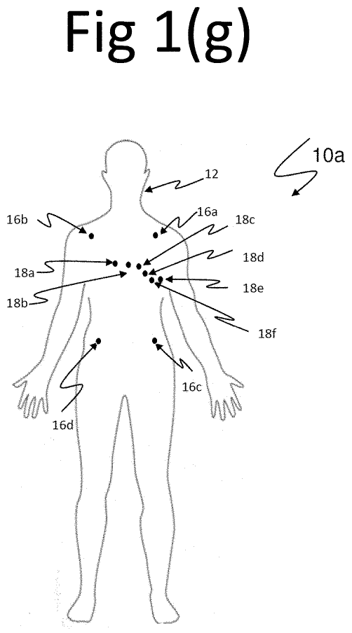

Referring now in detail to the various views of the drawings, there is illustrated in FIG. 1 a leadless wireless ECG measurement system for measuring of bio-potential electrical activity of the heart in a patient's body, which is designated generally by reference numeral 10 and is constructed in accordance with the principles of the present invention. The ECG measurement system 10 acquires “short-lead” ECG heart-signals from the body 12 of the patient and transmits them wirelessly to a receiving base station or unit 14. The receiving base station 14 transmits the heart-signals to an ECG monitor 16 for displaying meaningful information to a physician or technician.

As shown in FIG. 1(g), the standard ECG measurement system 10a includes a plurality of electrodes or contact points 16a, 16b and 16c disposed on the patient's body in order to obtain limb lead signals I, II, and III. The electrode or contact point 16a is attached to the patient's left arm or shoulder (LA). The electrode 16b is attached to the patient's right arm or shoulder (RA). The electrode 16c is attached to the patient's left leg or lower left abdomen (LL). The electrode 16d is attached to the patient's right leg or lower right abdomen (RL). In addition, there is illustrated in FIG. 1(g) a plurality of electrodes or contact points 18a, 18b, 18c, 18d, 18e and 18f which are attached to the chest or precordial area so as to obtain the pre-cordial lead signals V1, V2, V3, V4, V5 and V6. In summary, the standard 12-lead ECG system is defined by the following:

Lead I between LA and RA

Lead II between RA and LL

Lead III between LA and LL

Calculated augmented leads aVF, aVR, and aVL

V1: right 4th intercostal space

V2: left 4th intercostal space

V3: halfway between V2 and V4

V4: left 5th intercostal space, mid-clavicular line

V5: horizontal to V4, anterior axillary line

V6: horizontal to V5, mid-axillary line

Further, the ECG measurement system 10 of the present invention includes at least one multi-contact bio-potential electrode assembly 20 also disposed on the patient's body 12 which is comprised of an electronic patch layer 22 and a disposable electrode layer 24 to which the patch layer 22 is attached on top thereof.

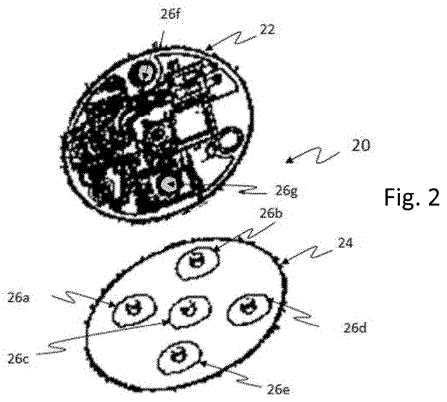

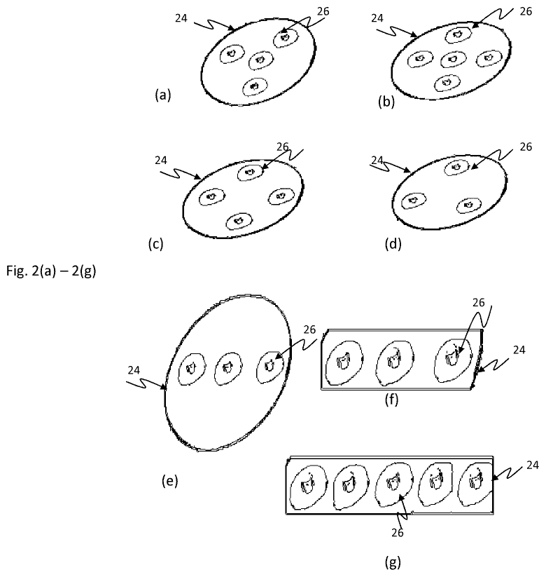

As can be best seen in FIG. 2, the electrode layer 24 includes a plurality (five) of contact or sensor points defining contacts 26a through 26e for engagement with the surface of the patient's skin for conducting the measurements. While the electrode layer 24 depicted with five contacts, it should be clearly understood by those skilled in the art that any number of contacts, more or less, may be used alternately as desired. However, it is preferable that three, four, or five contacts be utilized.

For reference, the ECG waveforms measured across the contacts 26a-26e of the electrode layer 24 are referred to as “short-leads” to distinguish them from the standard contact lead configuration “standard-leads” or “long-leads”, which are ECG measurements obtained from the electrodes 16a-16d and 18a-18f in FIG. 1(g). The electronic patch layer 22 includes all of the electronics for performing the measurements which can be integrated into a single micro-chip. The plurality of contacts (preferably two or three) represented by 26f and 26g on the electronic patch layer 22 can be used at calibration time to connect a plurality of remote long leads (preferably two or three) via an external or extended lead wire. When connected, contacts 26f and 26g act as the reference electrode of the patch to the standard long-lead electrodes which allow acquisition of referential unipolar channels. For example, to obtain a bipolar channel such as Lead I, one can subtract the two referential leads 26f-16a (LA) and 26g-16b (RA). Because this calibration step is done only briefly, the connecting lead to the standard electrodes locations can be to made to standard adhering electrodes placed at these locations, or preferably the extended leads have non-adhering conductive surface at its end that allows to measure the bio-potentials as it temporarily comes in contact with the surface of the skin for a short time (about 5-10 secs). The reference used for measuring the long leads can be different from the reference used to measure the short leads, or alternatively it can be the same reference contact point.

FIGS. 2(a) through 2(g) depicts various configurations of the disposable electrode layer 24 each having a plurality of contact points 26 arranged on its surface. The electrode layer 24 can be made from a conductive contact that is contacting the skin with an impedance matching conductive layer such as silver-silver chloride gel (AgCl) and an adhesive layer to attach to the skin, in conventional fashion as regular ECG electrodes would be, except the electrode layer 24 of the present invention has multiple contact points or contacts 26a-26e on the same electrode layer instead of a single contact point. A plurality of ECG channels can be measured from the single electronic patch layer 22 attached to the electrode layer 24 in the “short-lead” configuration.

As a wireless device, the electronic patch layer 22 has to conserve all available battery power during operation and that means using power efficient electronics components, and design, as well as computationally efficient software subsystem and algorithms. LED lights may be provided on the surface of the patch layer to communicate information to the patient such as to indicate connectivity link status, patch grouping status, or additionally to indicate alarm or operational status of the electronic patch layer. Further, LED color and/or LED blinking and/or speaker sound annunciation and/or display and/or wireless transmission of information can all be used in various ways as a means of indicating such as operational status or alarms or user instructions for calibration process or history of use and records of interest in memory.

This invention advantageously defines methods of calibration that emphasize ease of use by the user and enhance the usability of the leadless ECG device for better patient adoption and wearability comfort. Elderly patients are especially confused by multiple lead wires used during the calibration process and require an easier more familiar way of calibrating the device. The calibration per patient is necessary to define the transfer functions used to estimate long lead(s) (standard lead(s)) from the continuously acquired short lead(s) using the transfer function model obtained during the calibration step. In the description below we describe how we can achieve such enhancement of calibration process with benefits to human factors.

FIGS. 1(a) through 1(f) depict various preferred locations that a plurality of electrode assemblies 20 of the present invention may be placed on a patient's body 12. The multi-contact bio-potential electrode assembly 20 is preferably placed on top of the cardiac area on the surface of the patient's skin for the strongest possible signal with rich informational content, as shown in FIG. 1(b). One of the electrode assembly's plurality of contact points serves as a reference electrode contact point for the remaining contact points. Orthogonal “short-lead” directions produce signal content that is not redundant but independent and richer in its content.

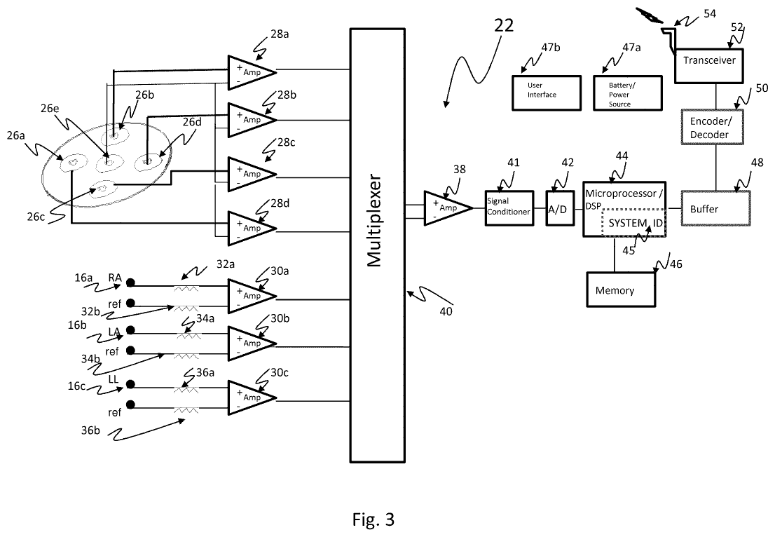

In another embodiment, a plurality of electrode assemblies 20 can be placed in proximity to the heart area but not necessarily directly on top of it, for example, next to or near the side of the heart, off from its central position, as shown in FIG. 1(a). Similarly, when being used for females, the electrode assembly can be placed more comfortably on top of the breast surface rather than underneath it. In still another embodiment the electrode assembly can be placed anywhere else on the skin, such as the torso, left shoulder, right shoulder, right or left hip area (FIG. 1(c)), or the back of the body such as the back shoulder or the back of the cardiac area on the skin. This flexibility of positional location of the electrode assembly attachment adds significant advantages in the comfort of use of the ECG measurement patch, thereby not restricting its placement. The leadless ECG measurement system of the present invention importantly does not require extension of lead wires (such as wires 32a, 32b shown in FIG. 3 to remotely distant electrodes during the continuous measurement phase, but only during the brief calibration phase.

The plurality of electrode assemblies (at least one) include an electronic patch layer preferably enabled with wireless transceivers for wireless communication with a remote wireless base station or for wireless communication across the plurality of electrode assemblies. In a preferred embodiment, the plurality of electrode assemblies and base station operate, preferably, in a known-in-the-art wireless mesh network topology (for example, as enabled using Zigbee wireless transceivers). In another preferred embodiment, the plurality of electrode assemblies and base station operate, preferably, in a known-in-the-art wireless star network topology (for example, as enabled by either Zigbee or Bluetooth wireless transceivers) enabling electrode assembly communication with a single base station. The mesh network topology has the added advantage of robustness and redundancy of communication pathway between the plurality of electrode assemblies and the remote base station as compared to the star network topology. In a mesh network, if communications between one or more wireless electrode assemblies with the remote monitor were lost, perhaps due to fading or interrupted RF channel pathway, then these electrode assemblies will try to communicate their data via any of the remaining communicating wireless electrode assemblies. A plurality of electrode assemblies can share information about the measured or estimated bio-potentials such as short-leads, or long-leads, or produced transfer function, or other useful information with each other or with the base station.

In FIG. 3, there is shown a block diagram of the electronic patch layer 22 of FIG. 2 connected with the plurality of contacts 26a through 26e of the disposable electrode layer 24. The electrical heart-signals defining the “short-lead” signals from the plurality of contacts 26a, 26b, 26d and 26e are supplied to a first input of a respective pre-amplifier stage 28a through 28d. The contact 26c provides a ground reference and is connected as a second input to each of the pre-amplifier stages 28a through 28d. Also, the heart signal defining the “standard-lead” or “long-lead” signal from the electrode 16a (FIG. 1(g)) and a reference signal are connected to a pre-amplifier stage 30a via external lead wires 32a and 32b. The heart signal defining the “standard-lead” or “long-lead” signal from the electrode 16b (FIG. 1(g)) and the reference signal are connected to a pre-amplifier stage 30b via external lead wires 34a and 34b. The heart signal defining the “standard-lead” or “long-lead” signal from the electrode 16c (FIG. 1(g)) and the reference signal are connected to a pre-amplifier stage 30c via external lead wires 36a and 36b.

The outputs of the pre-amplifier stages 28a-28d are fed to a high-gain amplifier 38 via multiplexer 40. The analog signals from the output of the high-gain amplifier 38 is fed to a signal conditioner 41 consisting of a high pass filter for baseline offset removal, a sampling anti-aliasing low pass filter, and a noise notch filter, and then to an a A/D converter 42 where they are filtered, sampled and converted to digital signals. These digitized signals are supplied to a microcontroller/Digital Signal Processor 44, which includes a System ID Processing device 45. Similarly, the outputs of the pre-amplifier stages 30a-30c are fed to the signal conditioner 41 and the A/D converter 42 via the multiplexer 40.

The System ID Model Processing device 45 performs a mapping function which relates the plurality of input measurements ECG “short-lead” waveforms vectors (from the pre-amplifier stages 28a-28d) with the plurality of output standard “long-lead” ECG waveform vectors (from the pre-amplifier stages 30a-30c). The identification system model structure, order and parameters describing the system relationship between the input “short-lead” signals and the output “long-lead” signals are stored in a memory 46. The microcontroller or digital signal processing unit 44 also processes commands and messages from the receiving base station 14 and executes programmed instructions stored in the memory 46. The processed digital ECG signals are then sent to a buffer 48 and an encoder/decoder 50 which are fed to a RF transceiver module 52 for transmission to the base station 14 via a low power built-in RF antenna 54. A battery/power source 47a is operatively connected to the various components for supplying DC power. A user interface 47b is provided which includes buttons, LEDs, or a display screen to permit a user to control and input various desired commands.

As should be clearly understood by those skilled in the art, for each such “short-lead” measurement, a minimum of three contacts are required to make a bipolar measurement (a positive contact, a negative contact, and a reference contact). The bio-potential at each of the contacts 26a-26e are presented to the amplifier 38 similar to the normal standard ECG measurement, however, the gain of such “short-lead” amplifier may be higher than the gain of standard lead amplifiers. Typical voltages detected across “short-leads” will be in the order of 10's to 100's of microvolts, while in standard “long-leads” (longer leads) the voltages will be generally from 1 millivolt to 10's of millivolts. It will also be understood the amplifiers 28a-28d and the high gain amplifier 38 can be configured to operate as a conventional instrumentation amplifier.

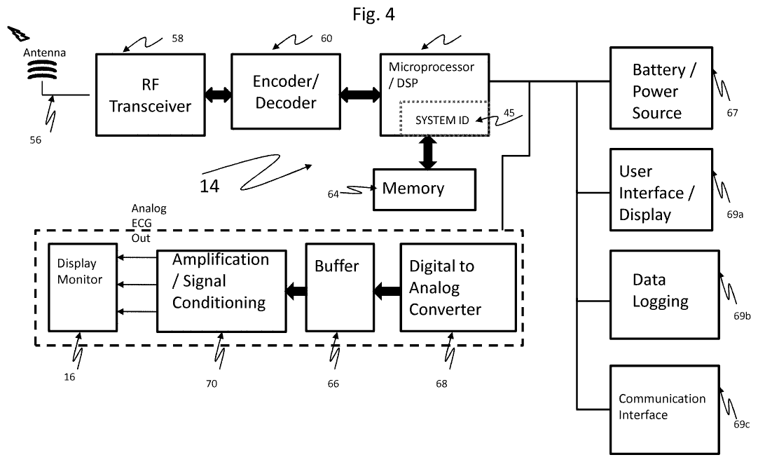

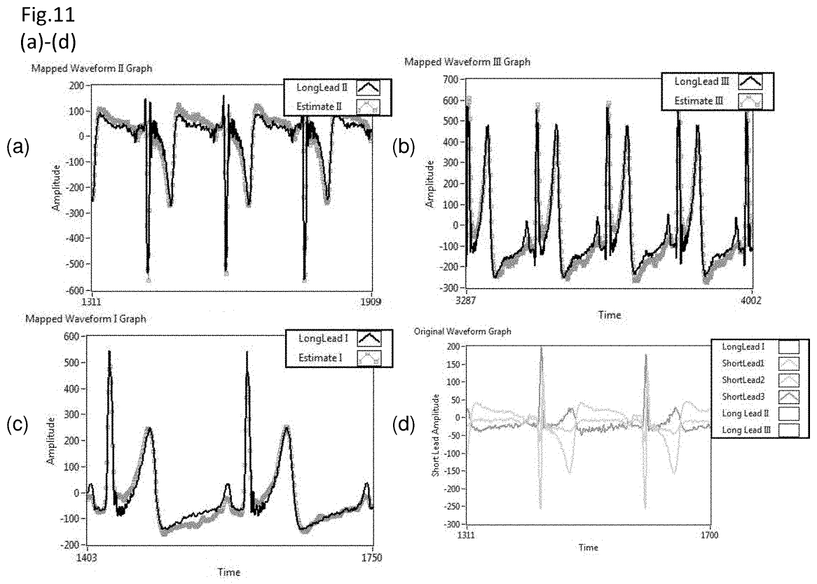

With reference to FIG. 4, there is shown a block diagram of the receiving base station 14 of FIG. 1. The base station 14 includes a low power RF antenna 56. In one embodiment, the base station 14 receives the transmitted “measured short-leads” digital ECG signals from the multi-contact electrode assembly 20. Optionally, the base station 14 receives the “measured long-leads” digital ECG signals from the electrodes 16a-16c (18a-18f) in FIG. 1(g) during calibration. In another preferred embodiment, the base station 14 receives the transmitted “estimated long-lead” digital ECG signals from the multi-contact electrode assembly 20. In FIGS. 11(a)-11(d), there are provided depictions of the mapping of short-leads to measured standard long-leads Lead I, Lead II, and Lead II producing a transfer function, preferably a linear state-space model in MISO structure, that outputs estimated long-leads based on short-leads only. The base station also includes a RF transceiver module 58 coupled to the RF antenna for receiving the processed digital ECG signals and feeds them to an encoder/decoder 60. A microcontroller/Digital Signal Processor (DSP) 62 is connected to the output of the encoder/decoder 60 for processing the digital ECG signals based on the programmed instructions for execution stored in a memory 64. The digital ECG signals from the microcontroller are supplied to a D/A converter 68 and a buffer 66 so as to convert the digital data to an analog form. The analog signals are sent to an amplifier/signal conditioner 70 and then to the ECG monitor 16 where the signals are treated as if they were generated from the standard ECG “long-lead” wire electrodes.

A battery/power source 67a is operatively connected to the various components for supplying DC power. A user interface device 69a is operatively connected to the microcontroller/DSP 62 to permit the user to control and input the various desired commands, wherein the user interface can also include a display monitor for displaying such received signals and derived valuable information as well as alerts and configuration information, and status information of the plurality of electrode assemblies. A data logger 69b allows saving of all system information to persistent memory/hard drive. A data communication interface 69c allows communication of all available digital information to external systems, including for example transmission of information via the internet or over TCP/IP protocols, or over cellular mobile networks, or local wireless networks.

While the System ID Model Processing device 45 is illustrated as being located in the microcontroller/DSP 44 of the electrode assembly 20, the System ID Processing device 45 shown in phantom may be located alternatively in the microcontroller/DSP 62 of the receiving base station 14 in order to reduce the size requirement of the power supply needed for the electronics in the patch layer 22 of the electrode assembly. The System ID Model Processing device 45 produces a system model which can be identified adaptively for each patient using two methods: (1) direct system identification and (2) blind system identification.

The direct system identification method is by application of conventional system identification methods for identifying adaptively the transfer function or mapping model relating the two waveform vectors between the “short-lead” measurement and the standard ECG “long-lead” measurement. By treating the “short-lead” signals as an input (stimulus) to a system with a certain transfer function characteristics that maps the input to a desired output (response) representing the standard “long-lead”, a variety of conventional system identification tools can then be applied for determining the optimal system order, system structure, and parameter values describing the system relationship adaptively.

While there are available many tools and system identification strategies, the preferred embodiment of the present invention utilizes a direct system identification which uses state space methods, and preferably in a multiple input single output (MISO) configuration, as more fully discussed below. The state-space MISO system identification allows implementation of system identification methods in the novel application of leadless ECG measurement, and extends the science of system identification to the realization of a feasible leadless ECG application.

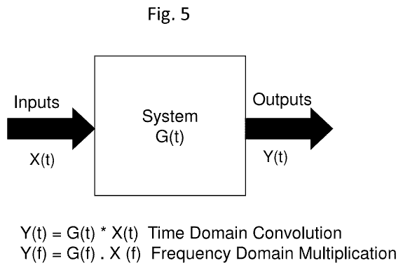

In FIG. 5, there is depicted a system transfer function G(t) which illustrates the relationships in the time and frequency domains between the inputs X(t) and outputs Y(t). As can be seen, in the time domain the inputs X(t) are convoluted with the transfer function G(t) to produce the outputs Y(t). On the other hand, in the frequency domain the inputs X(f) are simply multiplied in order to obtain the outputs Y(f). Equivalently, any linear transfer function can be represented in different linear models structures or forms including non-parametric models (impulse response, frequency response) or parametric models (such as state-space, transfer functions, and polynomial models including Auto-progressive AR, Auto-regressive with noise ARX, Auto-regressive Moving Average ARMA, Auto-regressive Moving Average with noise ARMAX, Box Jenkins, Output Error, Generalized Linear) that describes. In a preferred embodiment, the linear state-space model structure representation of the transfer function and state-space model identification function allows ease of adaptation of the system model using a change to its model order to optimize its approximation or estimation of the system outputs, given its system inputs.

The state space models (reference 4 to Kailath in the Appendix) are defined as

x(k+1)=Ax(k)+Bu(k)

y(k)=Cx(k)+Du(k)+n(k)

Where u(k), y(k), and x(k) are time series of real numbers representing the input, output, and state, respectively, of the system, and n(k) is time series of real numbers representing the noise term which is assumed to be independent of the input sequence u(k). A, B, C, and D indicate the coefficients vectors.

By using the Fourier transform on both sides of both equations of the state space model, we obtain the following, where sup. indicating superscript, and exp. indicates the exponent:

(exp.sup.jw)X(w)=AX(w)+BU(w)

Y(w)=CY(w)+DU(w)+N(w)

Where w is the frequency term, j indicates an imaginary number, and Y(w), U(w), X(w), N(w) are the frequency transformed output, input, noise, and state variables. The variables A, B, C, D indicate the coefficients vectors. Where

G(exp.sup.jw)=G(z) at z=exp.sup.jw=D+C((zI−A).sup.-1)B at z=exp.sup.jw and,

Y(w)=G(exp.sup.jw)U(w)+N(w)

is the frequency response function (FRF) of the system, I represents the identity matrix and sup.-1 represents a matrix inverse, sup represents superscript.

It will be noted that many other conventional System Identification methods exist and can provide substantially equivalent implementations to the state-space methods preferred in the present invention. These include (a) linear system identification (SYSID) methods, (b) nonlinear system identification methods, and (c) blind system identification methods. The background of each is discussed in details with algorithms describing prior art implementation details of such methods. The methods described apply for SISO (single input single output), MISO (multiple input single output), SIMO (single input (stimulus) multiple outputs (responses)), and MIMO (multiple inputs (stimuli) multiple outputs (responses)) configurations. This instant invention can equivalently, without loss of generality, use any of the other system identification methods mentioned below, and in any configuration of the inputs and outputs mentioned previously.

For example, as described in the references 1 through 6 listed in the Appendix which describe linear systems estimation and system identification methods, which are incorporated herein by reference, the linear SYSID parametric and non-parametric methods include:

AR

ARX

ARMA

ARMAX

Generalized Linear

Output Error

Box-Jones

Continuous transfer function

Discrete transfer function

Impulse realization

User defined model

Principal components subspace identification

Discrete frequency transfer function from a frequency response function

Continuous frequency transfer function from a frequency response function

Maximum likelihood methods

The nonlinear SYSID methods include:

Neural Networks

Fuzzy Logic

Volterra Series

Weiner models (LMS, or recursive least square based)

Wavelets analysis

Nonlinear state-space models

The blind system identification methods include:

Laguerre model based

Deconvolution methods

The system identification of the present invention can be either a SISO, if for example a single input channel was mapped to a single output channel, or, preferably, a MISO if the identified system was determined by mapping (or relating) multiple input channels (stimuli, measured “short-leads”) into a single output channel (response, standard “long-leads”). Multiple inputs mapping provides greater informational content and therefore a better mapping accuracy to the output. Alternatively, a MIMO configuration can be used where the input measured signals (“short-leads”) are used to calculate multiple output responses (standard “long-leads”) in a single application of system identification methods rather than multiple applications of multiple subsystem identifications. Alternatively, a SIMO configuration can be applied where a single “short-lead” is used as an input to determine system relationship with multiple outputs (standard “long-leads”).

Furthermore, the new calculated output leads from the primary systems identified can also be used as inputs (stimuli) into other secondary identified transfer functions relating them to other outputs (response, standard leads). This process can be again repeated for determining tertiary identified transfer functions, etc. as needed. However, with each such transfer function estimation, using estimates to calculate further estimates degrades the quality of the overall estimation. The overall final transfer function will be a multiplication (in the frequency domain) of all the primary and secondary stage transfer functions.

Recursive modeling builds towards an optimal total model, which involves predicting a model and using the predicted model output as input (stimulus) into predicting another model. To accomplish this, the first model has to be identified with high confidence and quality yielding excellent goodness of fit of model predicted outputs and actual outputs. Optimal models identified in the first stage will avoid the build up of errors and avoid rapid degradation of prediction quality in the identification of subsequent secondary models and the estimation of their responses.

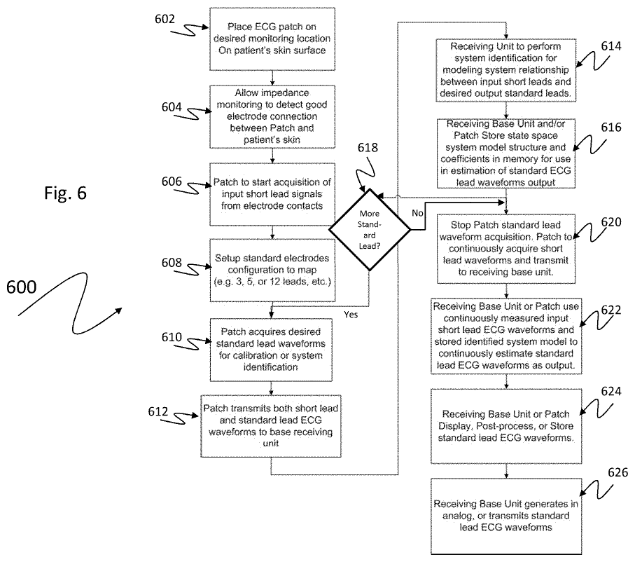

FIG. 6 shows a flow diagram 600 of calibration procedure for use where the standard “long-leads” are used as outputs for the direct system identification device. Initially, at step 602 the electrode assembly 20 is placed or attached on a desired location on the patient's skin, as shown in FIGS. 1(a)-1(f). At step 604, impedance monitoring is allowed to determine if a good electrical connection has been established between the electrode layer 24 and the patient's skin. At step 606, the electrode assembly 20 starts the ECG data acquisition of the input “short-lead” signals. Alternatively, the receiving base station 14 instructs all of the electrical contacts, such as 26(a)-26(e) on the electrode layer 24 in FIG. 2 to start the ECG data acquisition of the input “short-lead” signals. The step 606 may be initiated in response to a user prompt by means of any suitable user interface, such as device 69a associated with the receiving base station 14 in FIG. 4 or the user interface 47b on the electrode assembly 20.

At step 608, at least two of the standard electrodes 16(a)-16(d) and 18(a)-18(f) at the locations shown in FIG. 1(g) are used to obtain a calibration waveform substantially simultaneously to obtaining a “short-lead” signal during the process of calibration or system identification of the transfer function in the signal processor residing either in the receiving base station 14 or in the electrode assembly 20 is configured to map the number of “long-leads” used in the standard ECG system, such as 1, 3, 5, or 12 leads with the locations of the standard electrodes. Again, the configuration can be achieved through the user interface device 69a associated with the base station 14 or the electrode assembly 20. In step 610, the electrode assembly 20 acquires desired standard “long-lead” waveforms used for calibration or system identification. At step 612, the electrode assembly 20 transmits substantially simultaneously both the “short-lead” and the standard “long-lead” ECG waveforms to the receiving base station 14 for calculation of the transfer function system identification model.

In particular, in one embodiment, one extended lead wire connecting the electrode assembly 20 at the contact point 20a (or optionally 20b) to any of the standard electrode locations on the body 16(a)-16(d) and 18(a)-18(f) in FIG. 1(g), can provide means for obtaining a referential (unipolar) voltage potential between the two contact points. The process is repeated at least once to obtain a second referential voltage potential with a second standard electrode body location. The results of the two referential potential measurements allow the calculation of the differential potential between the two standard contact points. Such differential (bipolar) potential represents one of the standard ECG bipolar “long-leads”. If a “long-lead” is in fact unipolar, then a single referential measurement can represent such “long-lead”. The process of measurement of the “long-lead” is repeated until all “long-leads” are mapped in the calibration process. In another embodiment, at least two extended lead wires each connecting the electrode assembly 20 at the contact point 20a or 20b to at least any two of the standard electrodes 16(a)-16(d) and 18(a)-18(f) at locations on the body in FIG. 1(g), can provide means for obtaining a differential (bipolar) voltage potential between the two contact points for each extended lead wire. This embodiment accelerates the calibration mapping process. An extended lead wire is preferably external to the device, or it can be tethered and retractable into the patch electrode assembly, since it is used only during the calibration process, and containing conductive contact ends suitable for placement on the skin's surface for measuring bio-potential. Each time a referential or differential bio-potential is obtained for a “long-lead”, at least one “short-lead” bio-potential signal is also measured substantially simultaneously between any of the internal electrodes in the patch electrode assembly 20 that are in contact with the patient's skin. The contacts such as 32(a)-36(a) and 32(b)-36(b) in FIG. 3 are connected temporarily from the standard lead contact points 16(a)-16(c) to the measurement electronics of the patch layer 22 in the electrode assembly 20 so as to permit the acquisition of the standard “long-leads” and the short-leads” substantially simultaneously. The input “short-lead” ECG waveforms and the output “long-lead” ECG waveforms are alternately transmitted for a short period of time, e.g., about 5-10 seconds. This measurement is only needed for a brief time period, preferably a few seconds, for each “long-lead” in the desired configuration. The substantially simultaneously measured “long-lead” signals (outputs) and “short-lead” signals (inputs) are then processed by the signal processor to calculate the calibration transfer function or model, preferably in the form of a linear state-space model. In step 614, the base receiving station 14 performs system identification via the DSP 62 for modeling system relationship between measured input “short-lead” ECG waveforms and the desired output standard “long-lead” ECG waveforms.

The state space system model structure and coefficients are stored in the memory 64 associated with the base station 14 or in the memory 46 associated with the electrode assembly 20 in step 616 for use in the estimation of standard ECG “long-lead” waveforms. In step 618, it is determined whether there are additional standard leads to be measured. If the answer is “yes”, then the steps 610 through 616 are repeated. If on the other hand, the answer is “no”, the procedure will proceed to step 620.

In the step 620, acquisition of the standard “long-lead” waveforms from the electrode assembly 20 is stopped and the lead wires connected temporary between the standard lead contact points and the electrode assembly are then removed. Further, the electrode assembly 20 will then be configured to function in a continuous measurement operational mode, using the system identification model previously determined to continuously acquire “short-lead” waveforms and to transmit them to the receiving base station 14. In step 622, the receiving base station or the electrode assembly will use the continuously measured input ECG “short-lead” waveforms and the system identification model stored in memory to continuously estimate the output standard ECG “long-lead” waveforms. In step 624, the base station or electrode assembly will display, perform post-processing operations, or store the standard ECG “long-lead” waveforms. Finally, in step 626 the base station generates in analog form or transmits the standard ECG “long-lead” waveforms to the monitor 16 for displaying meaningful information to the physician or user.

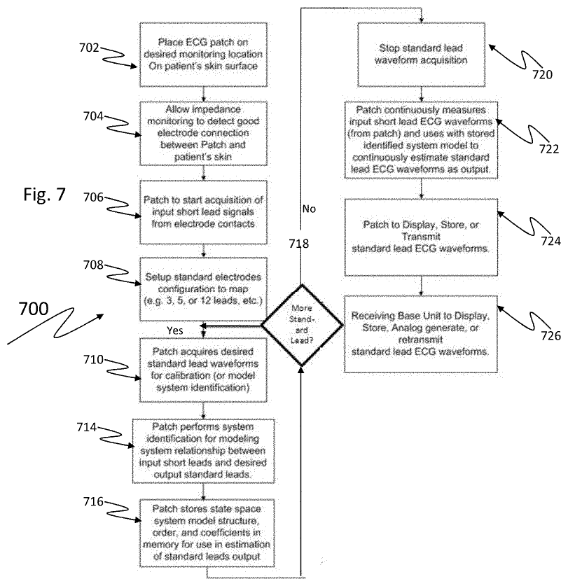

FIG. 7 depicts a flow chart 700 of an alternate calibration procedure for use where the standard “long-leads” are used as outputs for the direct system identification device. It will be noted that the calibration procedure of FIG. 7 is substantially identical to the procedure of FIG. 6, except that the system identification for modeling system relationship between the input “short-leads” and the desired output “standard-leads” is performed in the electrode assembly 20 instead of in the base station 14.

Specifically, at step 702 the electrode assembly is placed or attached on a desired location on the patient's skin. At step 704, impedance monitoring is allowed to determine if a good electrical connection has been established between the electrode layer and the patient's skin. At step 706, the electrical contacts on the electrode layer start the ECG data acquisition of the input “short-lead” signals. The step 706 may be initiated automatically by the electrode assembly 20 or in response to a user prompt by means of any suitable user interface associated either with the electrode assembly or the base station.

At step 708, the standard electrodes are set up and the electrode assembly is configured to map the number of leads used in a standard ECG system, such as 1, 3, 5, or 12 leads. Again, the configuration can be achieved through the user interface associated with the electrode assembly or the base station. In step 710, the electrode assembly acquires desired standard “long-lead” waveforms used for calibration or system identification. In particular, extended lead wires are connected temporarily from the standard lead contact points to the measurement electronics of the patch layer in the electrode assembly so as to permit the acquisition of the standard “long-leads” and the short-leads” substantially simultaneously.

In step 714, the electrode assembly performs system identification via the DSP for modeling system relationship between measured input “short-lead” ECG waveforms and the desired output standard “long-lead” ECG waveforms. The state space system model structure and coefficients are stored in the memory associated with the electrode assembly in step 716 for use in the estimation of standard ECG “long-lead” waveforms. In step 718, it is determined whether there are additional standard leads to be measured. If the answer is “yes”, then the steps 710 through 716 are repeated. If on the other hand, the answer is “no”, the procedure will proceed to step 720.

In the step 720, acquisition of the standard “long-lead” waveforms from the electrode assembly is stopped and the lead wires connected temporary between the standard lead contact points and the electrode assembly are then removed. Further, the electrode assembly will be configured to function in a continuous measurement operational mode, using the system identification model previously determined to continuously acquire “short-lead” waveforms. In step 722, the electrode assembly will use the continuously measured input ECG “short-lead” waveforms and the system identification model stored in memory to continuously estimate the output standard ECG “long-lead” waveforms. In step 724, the electrode assembly will display, perform post-processing operations, or transmit to the base station estimated standard ECG “long-lead” waveforms. Finally, in step 726 the base station will display, store, generates in analog form or re-transmits the estimated standard ECG “long-lead” waveforms to the monitor for displaying the waveforms and related meaningful information to the physician or user.

If degradation in quality of the reported standard lead signal estimation necessitates a recalibration step being necessary, due to a recommended time duration from a previous calibration being exceeded, or substantial change in impedance values monitored by the electrode assembly, or due to repositioning, removal, and replacement, of the electrode assembly. The system identification model can also allow reuse of previous calibration models, inputs and/or outputs stored in memory for comparative purposes of quality of signal, identifying changed components, or for accepting a previous or new calibration for continuous operation purposes.

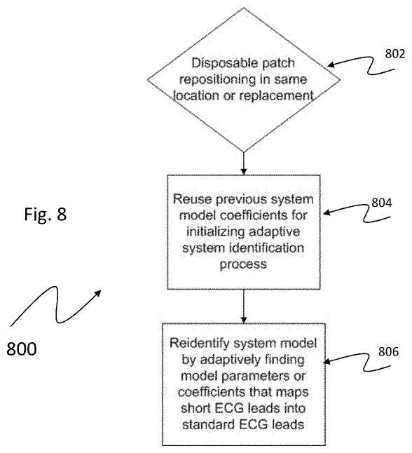

In FIG. 8, there is illustrated a flow chart 800 of a re-calibration procedure system model which re-uses previously identified system model parameters or coefficients as an initialization point to the new model system identification process. In other words, new system model parameters are found by beginning with the old system as the initialization point. In particular, in step 802, the disposable electrode layer is re-positioned in the same location on the patient's body. In step 804, the previous system model coefficients are re-used for initializing adaptive system identification process. In step 806, the previous system model created by adaptively finds model parameters or coefficients that maps the ECG “short-leads” into the standard ECG “long-leads” are re-identified.

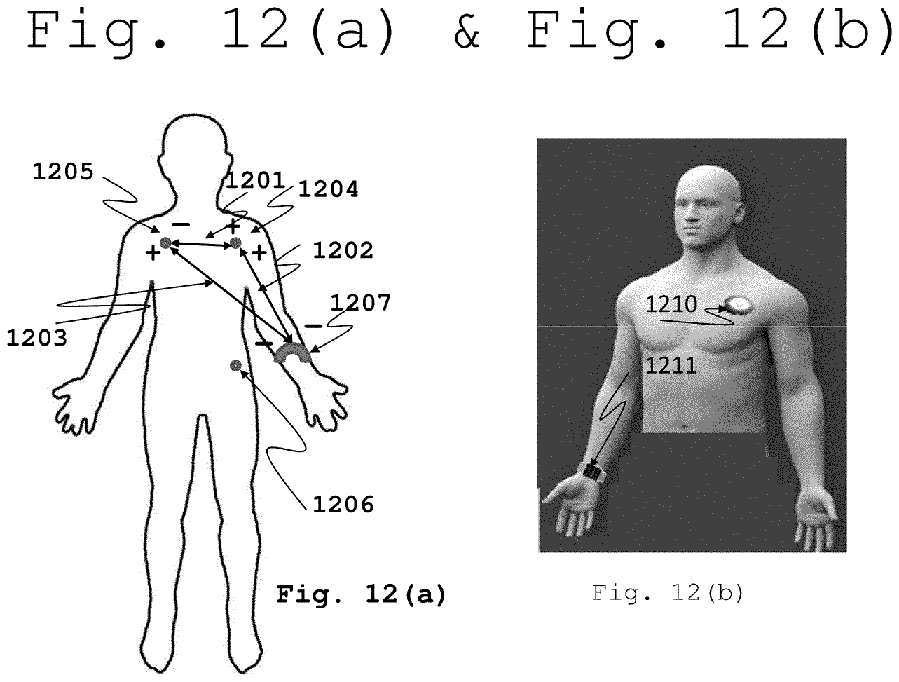

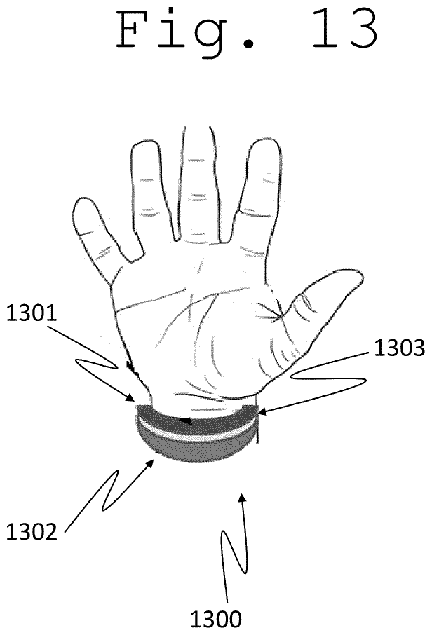

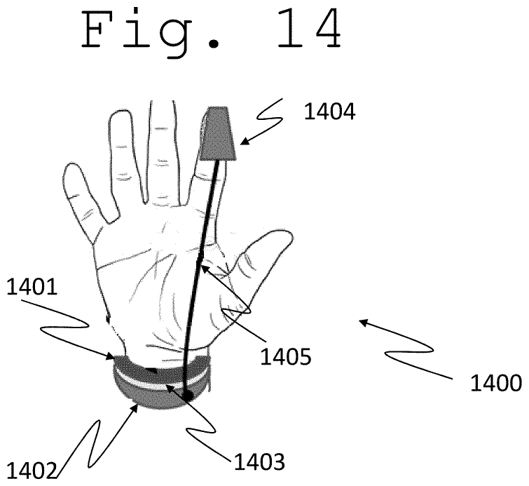

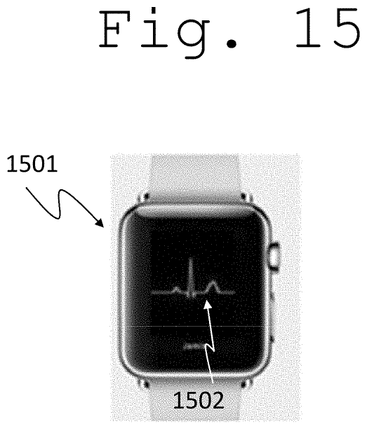

The following is in reference to FIGS. 12, 13, 14, 26, 27, 28, 29, and 30 relate to the discussion below.

FIG. 26 depicts an embodiment with finger calibration probe 2602 being tethered via a wire 2603 to the mobile device 2604 (smart phone or tablet) directly via a data acquisition interface unit 2605 that acquires ECG calibration signals (long leads) and communicate it to the mobile device. Additionally the patch 2601 is placed on patient's body (2607, or 2608) and communicates its acquired short leads ECG signals via wireless communication 2606, preferably Bluetooth, to the mobile device 2604.

FIG. 27 depicts an embodiment with finger calibration probe 2702 being tethered via a wire cable 2703 to the patch 2701 directly for data acquisition of calibration long leads ECG signals. The patch being placed on the patient's body (2707, or 2708) communicates calibration ECG and acquired short leads ECG signals to the mobile device 2704 (smart phone or tablet) via wireless communication 2706, preferably Bluetooth.

FIG. 28 depicts an embodiment with a wireless finger calibration probe 2802 used for data acquisition and wireless communication 2808 of calibration long leads ECG signals to the wearable smart patch 2801 and/or mobile device 2804 (smart phone or tablet) via wireless communication 2806, preferably Bluetooth. The wearable sensor patch 2801 being placed on patient's body (2807 or 2809) is used for data acquisition and communication of short lead ECG signals and/or estimated ECG signals to the mobile device.

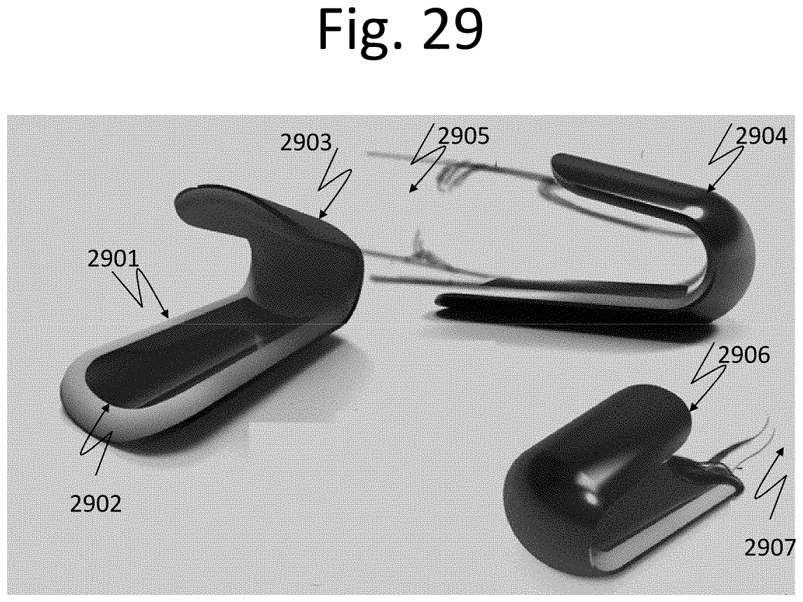

FIG. 29 depicts the finger calibration probe sensor 2904 being placed on patient's finger 2905 and used in the calibration process of mapping input ECG short leads from the wearable patch to output ECG long leads, preferably representing standard ECG leads and/or their components. The finger calibration probe can be either tethered with a wire cable 2907 to a ECG data acquisition device as in configuration 2906, or can contain the data acquisition electronics itself and communicates wirelessly the acquired signals as in configuration 2904. The finger calibration probe apparatus is constructed of at least an external conductive surface 2903, followed by an inner insulating layer 2901, followed by an inner conductive layer 2902 that is in contact with the patient's finger skin. Additionally, the finger calibration probe may contain internal data acquisition, battery, computational processing unit, memory, and wireless communication, preferably Bluetooth, electronic components to enable the data acquisition and wireless transmission of ECG data if not tethered to a separate data acquisition device.

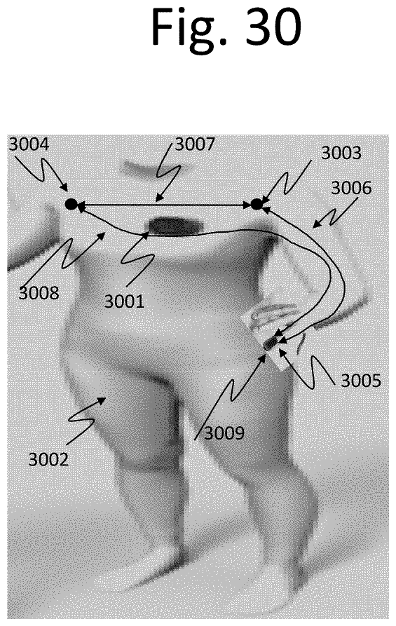

FIG. 30 depicts an wireless ECG patch 3001 being placed on patient's body 3002 during the calibration process steps for a standard ECG lead 3007 using first component ECG lead 3006 and second component ECG lead 3008 of a standard ECG lead 3007, making a closed Kirchoff's electric voltage loop. The first component 3006 is between a first standard contact 3003 and a common reference contact 3005 on the finger calibration probe 3009, and the second component 3008 is between a second standard contact 3004 and the same common reference contact 3005 on the finger calibration probe 3009. Wherein the standard lead biopotential is measured between contacts 3004 and 3003. This process is repeated to complete calibration for remaining standard leads.

In a preferred embodiment, the electrode layer is a dry electrode that is in contact with the patient's skin or tissue at a reference contact point. The dry electrode can be in the form of a conductive contact point, surface, line segment, perimeter, circumference, or wire that when in touch with the skin or tissue conducts the bio-potential electric current generated by the heart for measurement of the ECG. In an alternative embodiment, wet electrodes (similar to commonly known silver-silver chloride (Ag—AgCl) gel electrodes) are also possible to use for longer term contact, such as when making use of adhesive layer to secure attachment, however they are disposable in nature, and when removed may leave residue on the skin that requires cleaning. In a preferred embodiment, the ECG sensing device and the dry electrode(s) (used for short term), or wet electrode(s) (used for longer term), is/are adapted for attachment to a body part, including the front or back or sides of the torso, shoulders, chest area, neck, or hips whereas the attached device, including a patch, flat band, neck band, or arm band, is used for acquisition of ECG bio-potential waveforms between the electrode contact points.

In another preferred embodiment, as depicted in FIGS. 12(a), 12(b), 13, 14, 15, 16, 17, 18, and 19 the calibration probe is coupled to or includes wet (disposable) or preferably dry (reusable) electrode(s) is/are adapted for attachment to a flexibly moving body part, including a finger, arm, wrist, hand whereas the attached calibration probe device 1211, including a wrist watch 1501, a bracelet, a ring, finger probe, or a band, measures the electric bio-potential leads 1202 and 1203 between the electrode contact points (1207, 1204) and (1207, 1205), respectively, including between the flexibly moving body part's reference contact point 1207 and a plurality of other relative contact points on the rest of the body's surface skin or tissue 1204 and 1205, as defined by the standard ECG leads 1201 contact locations.

While the flexibly moving body part, with the ECG sensing device 1211 attached to it, is simultaneously in touch with the reference contact point (preferably using the interior electrode of a finger calibration probe 1207 in contact with the finger) and the (plurality of) relative contact points on the body 1204, 1205, 1206, preferably using the exterior electrode of a finger calibration probe 1207 in contact with the standard ECG contacts on the surface of the abdomen skin, the device 1211 acquires long lead ECG waveforms, or components of a standard lead, that are used for the calibration step(s) with the short leads acquired substantially simultaneously by the patch device 1702 (or equivalently 1601, 1801, or 1901) attached to the patient's body 1701 using the contact points of the electrode layer 1802 (upper view) or 1902 (bottom view). These short leads are used as model inputs while the long lead waveforms are used as model outputs in order to identify a transfer function model(s) that map the relationship(s) between said short lead(s) ECG waveform(s) as model input(s) and said long lead(s) ECG waveform(s) as model output(s). The said short lead(s) are acquired substantially simultaneously by an ECG sensing device 1210, including a wearable leadless wireless patch. The ECG sensing devices 1210 and 1211 are in communication with each other, and/or in communication with a base station to transfer such input and output waveforms and model parameters and other related useful information such as model order, type, structure, coefficients, and sampling rate. These identified models are later used with continuously acquired short leads inputs to estimate long lead output ECG waveforms.

The process is repeated during the calibration step(s) with the flexibly moving body part (i.e. finger, hand, arm, wrist) 1211 temporarily touching a plurality of contact points on the body, such as 1204, 1205, 1206. With each calibration iteration step, we identify the plurality of transfer function(s) for the plurality of long leads 1201, 1202, 1203 connecting the reference contact 1207 and the relative contacts point(s) 1204, 1205, 1206 defining the target standard leads, including Lead I (1201) (between contacts 1204 and 1205), Lead II (between contacts 1205 and 1206), and Lead III (between contacts 1206 and 1204), on the body. Each of the calibration steps define component waveforms (e.g. 1202, 1203) of the final standard or target lead(s) (e.g. 1201), such as limb leads I, II, III (representing bipolar biopotential between contact points located at Right Arm, Left Arm, and Left Leg or equivalently right shoulder, left shoulder, and left hip), and pre-cordial leads V1, V2, V3, V4, V5, and V6 (with contact points located on the chest around the heart area and representing a uni-polar potential relative to the right leg or equivalently right hip contact point).

The calibration probe's ECG sensing device 1211 can be in several embodiments including a wrist watch 1501, a bracelet, a finger probe, a ring, or a band has a conductive (preferably dry) electrode 1207 in the form of a contact point, surface, line segment, perimeter, or circumference on the exterior surface of the ECG sensing device, as depicted in FIG. 12(a), FIG. 12(b), FIG. 13, FIG. 14, and FIG. 15. The exterior conductive surface contact 1302 on the hand or wrist or arm attached sensing device 1211 is used to contact the relative points on the body 1204, 1205 to form an ECG lead 1201 bio-potential with the reference contact electrode on the interior conductive surface 1301 of the ECG sensing device 1207 attached to or in engagement with the skin or tissue (i.e Short leads). An insulating layer 1303 is in between the conductive layers 1301 and 1302. Alternatively an extension line 1405 tethered contact dry electrode 1404 can be attached to a patient's finger (facilitating reach and pointing), as depicted in FIG. 14, and can be connected to the ECG sensing device exterior contact electrode with conductive wire(s) cable. The extension dry electrode 1404 can be placed on a flexibly moving body part, including any of the patient's fingers (thumb, index, middle, ring, or pinky). This electrode is used temporarily for touching relative contact points 1204, 1205, 1206 during the calibration steps, and can be removed after calibration. Measured long and/or short leads 1502 can be displayed on the wrist mounted device 1501.

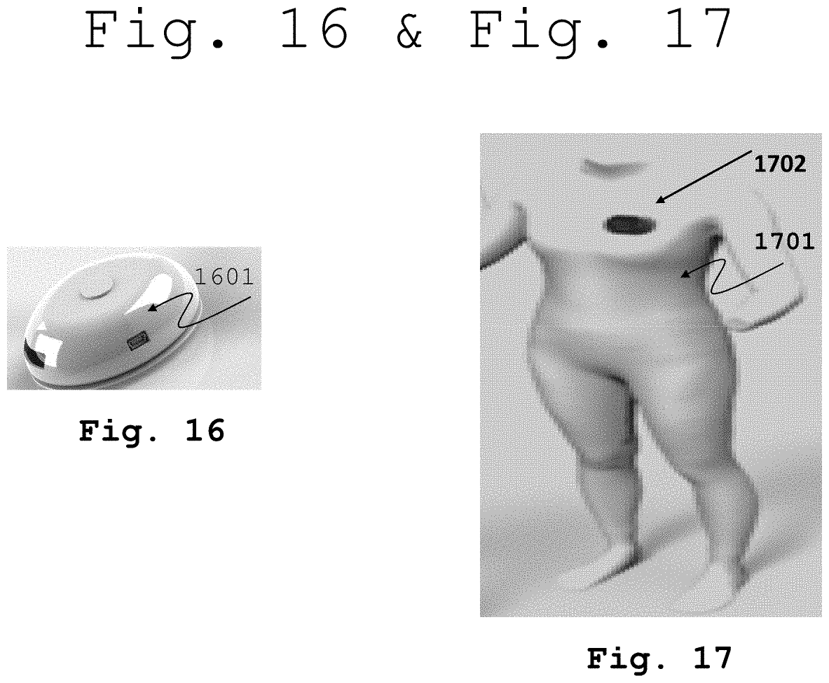

FIG. 16 depicts the leadless wireless ECG patch 1601, which is positioned in FIG. 17 as 1702 on the patient's body 1701.

In reference to FIG. 30 on the calibration process, the flexibly moving body part with the dry electrode (or its tethered extension) touches a first standard ECG lead relative contact point on the body. A bio-potential difference can be measured between the reference contact point, on the interior of the surface of ECG sensing calibration probe, and the relative contact points on the body using an electrode, preferably dry electrode, using the exterior of the ECG sensing calibration probe or its tethered extension. The bio-potential waveform is acquired between the two contact points, a reference contact and a relative contact, using the ECG sensing device. This bio-potential waveform will represent a first long lead waveform as a first model output. An ECG sensing device, a wearable ECG patch, measures the short lead(s) (as a first model input(s)) substantially simultaneously, and a processor is configured to compute and identify the first model transfer function coefficients that represents the relationship between the said first model input(s) and the first model output. Said transfer function identification is preferably performed using system identification methods, and more preferably using linear state space model identification. The first long lead waveform represents a first component waveform of a standard ECG lead waveform measurement. This first component waveform can be subsequently estimated continuously as an output waveform using the said identified (first model) transfer function and continuously acquired short lead(s) waveform(s) as model input(s).

The calibration process is then repeated with another relative contact point of the target standard ECG lead to obtain a second component waveform of a standard ECG lead waveform measurement. This bio-potential waveform will be measured as a second long lead waveform forming a second component waveform of a standard ECG lead waveform measurement. This second component can be subsequently estimated continuously using the (second) transfer function model and the continuously acquired short lead(s) waveform(s) as model input(s).

As we measure the standard lead that forms a Kirchoff's voltage loop with the first component long lead and the second component long lead waveforms (used as model outputs), we simultaneously measure the short distance ECG lead waveform(s) (used as model inputs). The model inputs and outputs are used to identify a transfer functions with system identification methods, preferably linear state space model, which maps the relationship between the inputs and outputs as a calibration transfer function. Once identified, the transfer function (model) is then used in a second step to estimate the long lead(s) model output(s) (components of the standard ECG lead(s)) given the identified model (i.e. transfer function's structure and coefficients) and the short lead(s) as model input(s). The first component waveform can then be continuously estimated using the short leads continuously acquired and the first transfer function.

The second component waveform can then be continuously estimated using the short leads continuously acquired and the second transfer function. Whereas, the mathematical difference between the first estimated component waveform and second estimated component waveform of the standard lead will represent the estimated standard lead waveform in its defined standard form or morphology. The three leads (target standard lead, first component lead, and second component lead) form an electrical circuit with a Kirchoff voltage loop using the skin impedance between the standard lead's contact points and the reference contact point on the ECG sensing device attached to the flexibly moving body part.

The calibration process, as described in FIG. 30, is repeated until all target standard leads component waveform(s) are measured, and used as model outputs along with, substantially simultaneously, measured short lead(s) to identify or define transfer function(s) model(s). In a subsequent step, the continuously acquired short lead(s) are then used as input(s) with the previously identified transfer function(s) to estimate the long lead(s) component waveform(s). At least two components waveforms (forming the Kirchoff loop with the target standard lead) estimates are then used in estimating each of the target standard lead. The two components estimates are subtracted to form the desired standard lead per Kirchoff voltage loop law as depicted in FIG. 12(a).

A subsequent recalibration between the same input contact points and output contact points can be performed for optimizing the model parameters, for example, when tissue characteristics are varied such as due to hydration status or pain or stress level. In an embodiment, the recalibration step for the model coefficient can be initialized from the previous identified model coefficients for faster convergence on new model parameters yielding an optimized performance.

In another embodiment, as depicted in FIGS. 13 and 15, the contact point(s) of the body part attached device used for monitoring ECG is/are represented by the inner surface segment or circumference of the device attached to the body part, including that of a wrist watch, a wrist band, bracelet, hand band, finger ring, arm band, leg band, ankle band or bracelet, neck band, or necklace in contact with the skin or tissue. Preferably the watch is used to measure the ECG from the reference contact point that is placed either at the inner surface of the watch's head in contact with the skin or tissue, or using the inner surface of the watch's band in contact with the skin or tissue. Preferably the watch can be used to display the various ECG leads information including the waveform, measured parameters, heart rate, patient information, detected abnormalities, time since last abnormality, battery status of the ECG sensing device, alarm information, configuration information, and relevant clinical diagnostic information and reference information.

To detect the need for recalibration due to hydration status or tissue characteristics variation, a transfer function model between the short leads is identified, preferably using system identification methods defined herein, and preferably using state space linear system model. The model uses continuously acquired short lead(s) waveforms as input(s) and other short lead(s) as output(s) and finds the coefficients of the model mapping the short lead(s) input(s) to output(s). This process is repeated intermittently periodically to examine the degree of variation of the identified model coefficients from previous iteration and to determine if body characteristics changed significantly from previous calibration point to trigger a prompt for the user to recalibrate the transfer function defining the relationship between the long lead(s) with the short lead(s) from it's previous baseline point. The short lead input signals can be used with the transfer function during calibration time to estimate the output short leads. Then we can compare these estimated output short leads and actual short lead output signals to measure a degree of correlation among them and compare new correlation coefficients with previous correlation coefficients. If a variation greater than a desired target correlation coefficient threshold is detected then recalibration of short lead/long lead models is required by the user to maintain long lead estimation accuracy. Alternatively, one short lead can be used as input signal and another short lead as an output signal to identify a transfer function model between them during the calibration step. The transfer function identified by nonlinear or linear system identification methods, preferably linear state space models, Furthermore, for greater accuracy and convergence of the identified transfer functions, each recalibration of a model relationship between the short lead input signal and short lead output signal can be preferably initialized from the previous calibration coefficients to allow for faster convergence.

The body or the body part attached ECG measuring devices described herein, including patch, bracelet, wrist watch, hand band, neck or arm band, necklace or finger ring, are adapted to measure at least one of the short lead ECG waveforms and the long lead ECG waveforms.

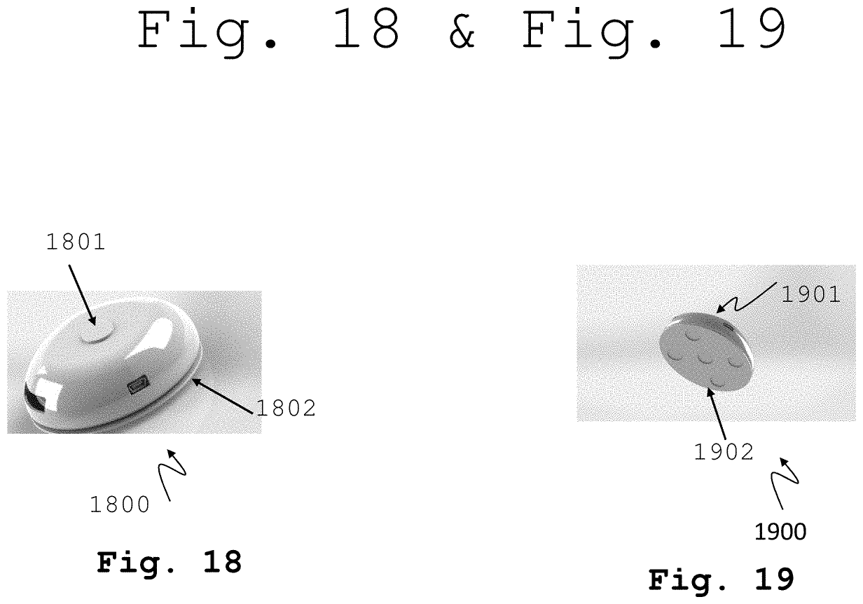

In yet another embodiment, as depicted in FIGS. 18 and 19, the ECG sensing device, in the form of a patch or flat band or arm band, is attached to the body, preferably upper front or side torso or chest area, for continuously acquiring short lead ECG signals using the electrode layer contact points on the interior of the device engaging the body in attachment, while the exterior surface of the device is enabled with dry contact electrode(s) layer representing a reference contact on the device that the patient uses to touch their left hand, finger, wrist, or arm and their right hand, finger, wrist, or arm to establish calibration of the short leads with a standard lead, including lead I. In this embodiment, the estimation of the standard lead is performed by subtraction of two estimated component waveforms. The first component waveform is between the left hand, finger, wrist, or arm relative contact point and the reference contact on the ECG sensing device. The second component waveform is between the right hand, finger, wrist, or arm relative contact point and the reference contact on the ECG sensing device. In a first step, we obtain a first calibration transfer function model between the short lead(s) input(s) from the patch and the first component waveform from the long lead being calibrated to as an output signal. In a second step, we obtain a second calibration transfer function model between the short leads inputs from the patch and the second component waveform from the long lead being calibrated to as an output signal. In a third step, we use the continuously acquired short lead waveforms as model input(s) into the first transfer function model to estimate continuously the said first component waveform as first model output, and also, substantially simultaneously, we use the same continuously acquired short lead waveforms as model input(s) into the second transfer function model to estimate continuously the second component waveform as a second model output. In a fourth step, we subtract the estimated first component calibration signal and estimated second component calibration signal (model outputs) to yield the estimated target standard lead, including lead I, between the right arm (right hand, finger, wrist, or arm) and the left arm (left hand, finger, wrist, or arm).

In summary, we described a system a biopotential measurement and calibration system for leadless electrocardiographic (ECG) measurement of electrical activity of the heart in a subject's body, the system comprising: at least one multi-contact bio-potential electrode assembly adapted for attachment to the subject's body, said electrode assembly being formed of an electronic layer and an electrode layer; said electrode layer having a plurality of contact points for engagement with the surface of the subject's body and configured to measure short-lead ECG signal(s) in response to electrical activity of the heart; a calibration probe having an interior conductive surface and an exterior conductive surface, wherein said calibration probe is capable of sensing one or more ECG biopotential calibration long leads when (1) said interior conductive surface is in contact with a patient's finger or wrist, and (2) said exterior conductive surface is in contact with patient's body; and a processing unit configured to produce a transfer function during calibration based on the measured short-lead ECG signal(s) from said plurality of contact points and said one or more calibration long leads; and thereafter uses said transfer function to compute estimated calibration long-lead ECG signal(s) based on the measured short-lead ECG signal(s) from said plurality of contact points. In an embodiment, said ECG biopotential calibration long lead represents a component of a closed Kirchoff's voltage loop with a standard ECG long lead. In an embodiment, said ECG biopotential calibration long lead represents a standard ECG long lead signal. In an embodiment, the system further comprising a monitor in communication with at least one multi-contact electrode assembly, wherein said monitor is configured to receive at least one of said transfer function, measured short lead ECG signal(s), measured or estimated calibration long lead ECG signal(s), measured or estimated long lead ECG signal(s), measured or estimated standard long lead ECG signal(s), for displaying said ECG signal(s) and other meaningful information. In an embodiment, said electrode assembly is coupled to a transceiver unit to receive at least one of said transfer function, measured short lead ECG signal(s), measured or estimated calibration long lead ECG signal(s), measured or estimated long lead ECG signal(s), measured or estimated standard long lead ECG signal(s), for processing said ECG signal(s) and other meaningful information. In an embodiment, said leadless ECG system is wireless, said electronic layer is coupled to or includes a transceiver unit for transmitting and receiving wireless communications with a base station or a monitor, and said base station or monitor includes a wireless transceiver for transmitting and receiving communications with said contacts of the electrode assembly, wherein said wireless communications received by said wireless transceiver include at least one of said transfer function, measured short lead ECG signal(s), measured or estimated calibration long lead ECG signal(s), measured or estimated long lead ECG signal(s), or measured or estimated standard long lead ECG signal(s). In an embodiment, said leadless ECG system is wireless, and said calibration probe includes a transceiver unit for transmitting and receiving wireless communications with a base station or a monitor, and said base station or monitor includes a wireless transceiver for transmitting and receiving communications with said calibration probe, wherein said wireless communications received by said wireless transceiver include at least one of said transfer function, measured short lead ECG signal(s), measured or estimated calibration long lead ECG signal(s), measured or estimated long lead ECG signal(s), or measured or estimated standard long lead ECG signal(s). In an embodiment, said calibration probe is wireless, and said calibration probe includes a transceiver unit for transmitting and receiving wireless communications with a base station or a monitor, and said base station or monitor includes a wireless transceiver for transmitting and receiving communications with said calibration probe, wherein said wireless communications received by said wireless transceiver include at least one of said transfer function, measured short lead ECG signal(s), measured or estimated calibration long lead ECG signal(s), measured or estimated long lead ECG signal(s), or measured or estimated standard long lead ECG signal(s). In an embodiment, said at least one multi-contact bio-potential electrode assembly is in communication with at least a second multi-contact bio-potential electrode assembly, said communications including at least one of said transfer function, measured short lead ECG signal(s), measured or estimated calibration long lead ECG signal(s), measured or estimated long lead ECG signal(s), measured or estimated standard long lead ECG signal(s), and other meaningful information. In an embodiment, said processing unit is disposed in said electronic layer of said electrode assembly. In an embodiment, said processing unit is disposed in said calibration probe. In an embodiment, said processing unit is disposed in said base station or monitor. In an embodiment, said transfer function is identified using a system identification method. In an embodiment, said transfer function is identified using a system identification method employing a linear state-space model. In an embodiment, said processing unit determines the need for a new calibration step to re-identify the transfer function. In an embodiment, transfer function computes estimated long-lead ECG signal(s) based on at least one other estimated long-lead ECG signal(s), measured long-lead ECG signal(s), or measured short-lead ECG signal(s) from said plurality of contact points. In an embodiment, said processing unit employs signal processing and analysis on said measured and estimated ECG signal(s) to detect and indicate abnormalities in ECG rhythm or patient's health state. In an embodiment, said electronic layer includes a plurality of electrical contacts for attaching a plurality of extended lead wires for measurement of a plurality of long-lead signal(s). In an embodiment, said long-lead signal(s) represent standard ECG lead(s) with standard ECG electrode locations on the body. In an embodiment, said electrode assembly is placed on top of or at proximity to the cardiac area, including next to or near the heart. In an embodiment, said electrode assembly is placed within proximity to a fetal area of the maternal abdomen, and the bio-potential electrical activity contains fetal ECG (fECG) for monitoring fetal heart electrical activity. In an embodiment, the bio-potential electrical activity represents evoked potentials (EVP) of any tissue. In an embodiment, said electrode assembly is placed in contact with the heart muscle and the bio-potential electrical activity represents cardiac electrograms (EGM) for monitoring heart muscle activity.

In summary, we described a method whereby a biopotential measurement and calibration method for leadless electrocardiographic (ECG) measurement of electrical activity of the heart in a subject's body, comprising: providing at least one multi-contact bio-potential electrode assembly adapted for attachment to the subject's body, said electrode assembly being formed of an electronic layer and an electrode layer, said electrode layer having a plurality of contact points for engagement with the surface of the subject's body; providing a calibration probe having an interior conductive surface and an exterior conductive surface, said interior conductive surface in contact with a patient's finger or wrist, said exterior conductive surface in contact with the patient's body; measuring, using the at least one multi-contact bio-potential electrode assembly, first short-lead ECG signal(s) in response to electrical activity of the heart;

sensing, using the calibration probe, one or more ECG biopotential calibration long leads; producing, using a processing unit, a transfer function during calibration using said measured first short-lead ECG signal(s) and said ECG bio-potential calibration long leads; and computing, using said transfer function, estimated calibration long-lead ECG signal(s) based on second measured short-lead ECG signal(s) from said plurality of contact points. In an embodiment, Said ECG biopotential calibration long lead represents a component of a closed Kirchoff's voltage loop with a standard ECG long lead. In an embodiment, said ECG biopotential calibration long lead represents a standard ECG long lead signal. In an embodiment, said transfer function is identified using a system identification method, preferably employing a linear state-space model. In an embodiment, said system identification method initializes from a previous said transfer function. In an embodiment, Said processing unit determines the need for a new calibration step to re-identify the transfer function. In an embodiment, said transfer function computes estimated long-lead ECG signal(s) based on at least one other estimated long-lead ECG signal(s), measured long-lead ECG signal(s), or measured short-lead ECG signal(s) from said plurality of contact points. In an embodiment, said processing unit employs signal processing and analysis on said measured and estimated ECG signal(s) to detect and indicate abnormalities in ECG rhythm or patient's health state. In an embodiment, Said electronic layer includes a plurality of electrical contacts for attaching a plurality of extended lead wires for measurement of a plurality of long-lead signal(s). In an embodiment, said long-lead signal(s) represent standard ECG lead(s) with standard electrode locations. In an embodiment, said electrode assembly is placed on top of the cardiac area on the surface of the subject's skin. In an embodiment, said electrode assembly is placed at proximity to the cardiac area, such as next to or near the side of the heart. In an embodiment, said electrode assembly is placed within proximity to a fetal area of the maternal abdomen. In an embodiment, said electrode assembly is placed on at least one of: the left shoulder area, left arm, left side, right shoulder area, right arm, right side, upper frontal area, upper frontal abdominal area, abdominal area, upper dorsal area, or dorsal area of the subject's body. In an embodiment, said system is employed in conjunction with a standard ECG measurement system to improve performance against leads providing a noisy signal or disconnected leads. In an embodiment, said estimated long leads are converted into analog output signal(s). In an embodiment, the bio-potential electrical activity represents an electroencephalogram (EEG) for monitoring brain activity, or represents an electromyogram (EMG) for monitoring muscle activity, or represents fetal ECG (fECG) for monitoring heart activity, or represents evoked potentials (EVP) of any tissue, or represents a cardiac electrogram (EGM) for monitoring heart muscle activity, or represents an electroneurogram (ENG) for monitoring nerve activity. In an embodiment, the variation from measurement time to calibration time in the biopotential estimation error for any lead is used to evaluate the health state of the patient, medication effect on patient, or a need for recalibration.

In another embodiment, a lead is temporarily connected to the ECG sensing device at a third contact point, including standard left leg (leg, or hip) contact point, using either a dry or wet electrode, whereby in a calibration step a third waveform component is measured and used as an output signal of a model calibrated to the input short lead(s) signal(s) with a third transfer function model to identify the model parameters and coefficients, preferably using system identification methods, preferably using linear state-space system identification. In a subsequent step, the continuously measured short lead(s) from the patch are used as inputs to the said identified third transfer function model to continuously estimate the third component waveform. The continuously estimated third component waveform is subtracted from the continuously estimated first waveform component waveform to estimate a standard lead waveform, including lead III.

The continuously estimated third component waveform is subtracted from the continuously estimated second component waveform to estimate a standard lead waveform, including lead II.

It is understood by those skilled in the art that the order and polarity of measurements for Lead I, II, or III, or any of their component leads, can be done in any order to complete the Kirchoff voltage loop defining the circuit path inclusive of leads I, II, III.

In yet another embodiment, as depicted in FIGS. 18 and 19, the ECG sensing device, in the form of a wearable ECG sensing patch or flat band or arm band, is attached to the body, preferably upper front or side torso or chest area, for continuously acquiring short lead ECG signals using the electrode layer contact points on the interior of the device engaging the body in attachment. The exterior surface of the device is enabled with dry contact electrode(s) layer representing a reference contact on the device that the patient uses to touch their left hand, finger, wrist, or arm and their right hand, finger, wrist, or arm to establish calibration of the short leads with the reference long lead. Depending on where the patch is positioned on or off a standard lead contact location, the reference long lead can represent either a standard lead or a component waveform of a standard lead, respectively. One finger, hand, wrist, or arm, preferably left, touches the reference contact on the device while the other finger, hand, wrist, or arm, preferably right, touches a standard contact point. Whereas the left and right arm act as a flexibly moving conductive surfaces connecting different body contact points on the skin or tissue and the reference contact. A bio-potential is then measured between any of the Short lead contact(s) (on the electrode layer) underneath the device and the reference contact point which is in short circuit with one hand (e.g. left or right) which is in turn in short circuit with the other hand (e.g. right or left) which in turn is in short circuit with the target standard contact point. Therefore the biopotential is measured between any of the short leads contact points and the target standard contact point. In order for the ECG signal to be conducted across the two arms as opposed to through other higher impedance pathways through the skin, the total impedance through the arms need to be less than the impedance, or preferably short circuit, through other skin pathways, which will make that path the path of least resistance for conducting the ECG biopotential. The impedance of the path along the two arms can be reduced using a conductive metal wire line between the two arms, preferably copper, flexibly extended between the left and right arms, wrists, hands, or fingers contact points. In this embodiment we make use of the fact that the two hands can mechanically extend to connect various standard leads contact points and the ECG sensing device's reference electrode, creating a natural calibration lead wire.

In yet another embodiment, the ECG bio-potential sensing device between the electrode contact points adapted for attachment to a body part, including the front or back or sides of the torso, shoulders, chest area, neck, or hips whereas the attached device, including a patch, flat band, neck band, or arm band is enabled with two dry contacts electrodes on its exterior surface. During calibration step, the patient touches the dry contact electrodes with the right and left fingers, hands, wrists, or arms simultaneously and the long lead representing standard lead I is measured between the two dry contact electrodes. The standard lead I is used as model output along with substantially simultaneously acquired short lead(s) as model input(s) to define the transfer function relationship of the model identified preferably using system identification methods and preferably using state space linear system models. The benefits of such state space linear models is that they are adaptive to any orientation of placement of the patch or band and thus renders their ability to estimate the output long lead in sensitive to placement orientation on the body across placements relative to the standard leads. Similarly because they are adaptive in nature, they are insensitive to amplitude variations due to electrodes sensitivity variations across placements or electrode type or production lot to lot variability.

In all of the discussion herein, the reference to a transfer function is preferably defined using a linear or nonlinear system identification methods, as described herein, and preferably using a state-space linear system model as described in herein as well. Either a single input single output (SISO) model, or a multiple input single output (MISO) model, or a single input multiple output (SIMO) model, or a multiple input multiple output (MIMO) model can be used.

User error control during the calibration process is critical to detect and fix, such as misplacement of the flexibly moving body part on the wrong standard contact point, or confusing left from right arm, for example. User calibration error can be detected and controlled by monitoring the direction of the R wave of the QRS complex of the ECG waveform, and to determine whether that should be positive or negative relative to the ECG waveform baseline. Also user calibration error can be detected and controlled by comparison of the acquired or estimated ECG waveform with other acquired or estimated ECG waveform or reference template waveforms to determine if comparison output has a sufficient variance above a threshold acceptable level to warrant us to alert the user to a potential calibration error or problem and the need to repeat the calibration step. Similarly, partial or full detachment of the short leads contact points from the body can be detected with loss of signal and alerting can be established. Furthermore, the device can be equipped with an accelerometer for detection of patient activity and adaptive filtering of motion artifacts from the ECG short leads or estimated ECG long leads. Motion artifacts can commonly affect the ECG sensory signal and can be adaptively filtered by using known in the art adaptive filters such as least mean square (LMS) filter, or normalized least mean square (NLMS), or Recursive Least Square (RLS), or QR decomposition based RLS (QR-RLS) adaptive filters with the motion activity signal (e.g. measured using the accelerometer) used as the noise input and the motion corrupted measured or estimated ECG as the desired input, and the output representing the desired motion-filtered ECG waveform. Also, during calibration process, the correlation of an estimated output signal from the model and a measured output signal from the calibration lead or its components can indicate acceptability of the calibration process. If the correlation coefficient was above a threshold (for example, greater than 90%), then the calibration process may be indicated to be repeated.

The discussion of systems and methods above on leadless wireless ECG application can be similarly extended and applied to other electrophysiologic (EP) waveforms, including EEG or EMG or neural monitoring, equivalently. Electrophysiologic signals can vary in frequency content and band, however, the same processing methods and steps apply on other

Fetal and Maternal ECG:

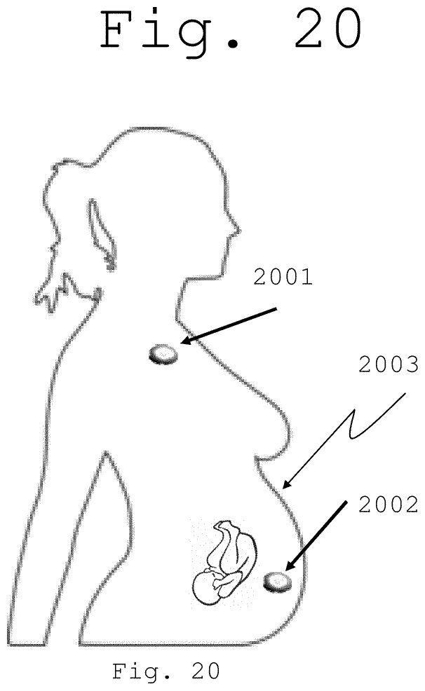

In reference to FIGS. 20, 21, and 22 depict preferred embodiments of monitoring configurations by placement of either two wearable sensing ECG patches, or a single wearable sensing ECG patch and an extension (tethered) lead wires, on a pregnant woman to measure mixed maternal and fetal ECG from an abdominal area skin surface contact points, and substantially maternal only ECG from across contact points on skin surface area further away such as the shoulder area of the mother. If two patches 2001 and 2002 are used on a pregnant mother 2003, as depicted in FIG. 20, then the two patches are preferably in communication with each other, in either wired or preferably wirelessly communication link, or alternatively both patches are in wireless communication with a common base station, for measuring the respective ECG waveforms substantially simultaneously from the short leads of each patch 2001 and 2002.

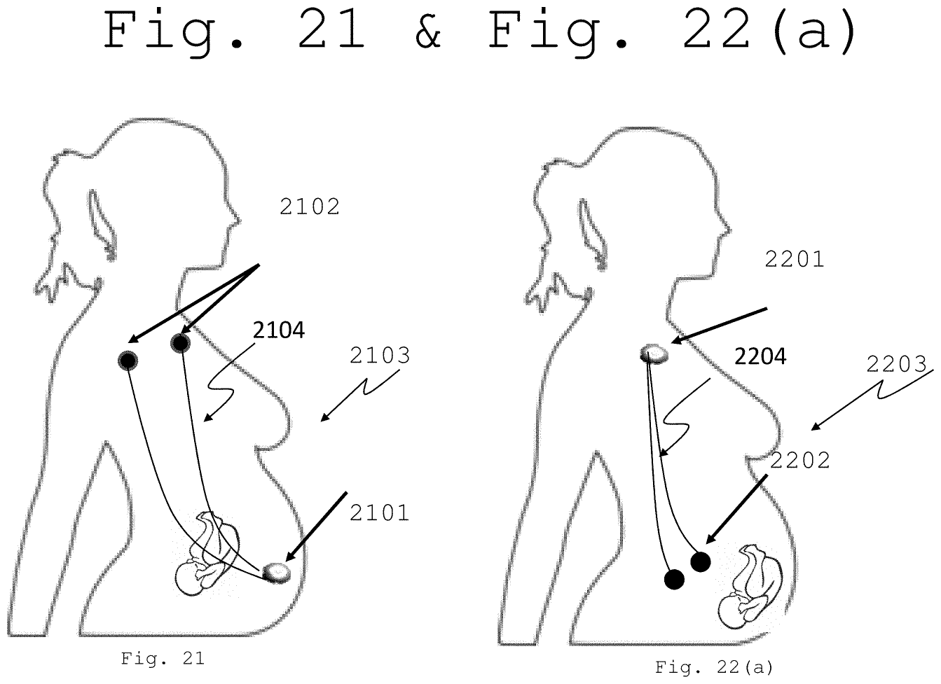

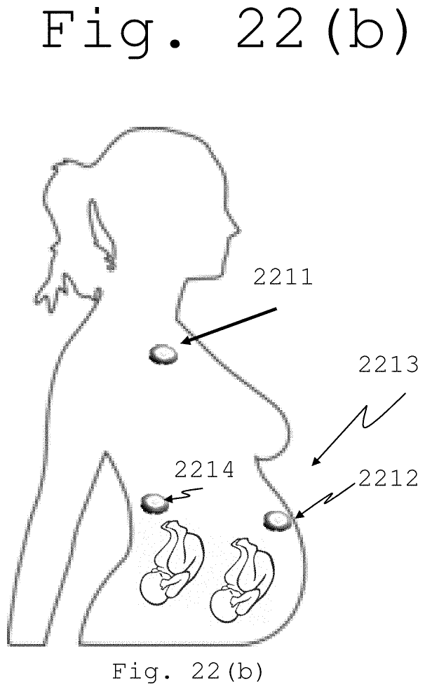

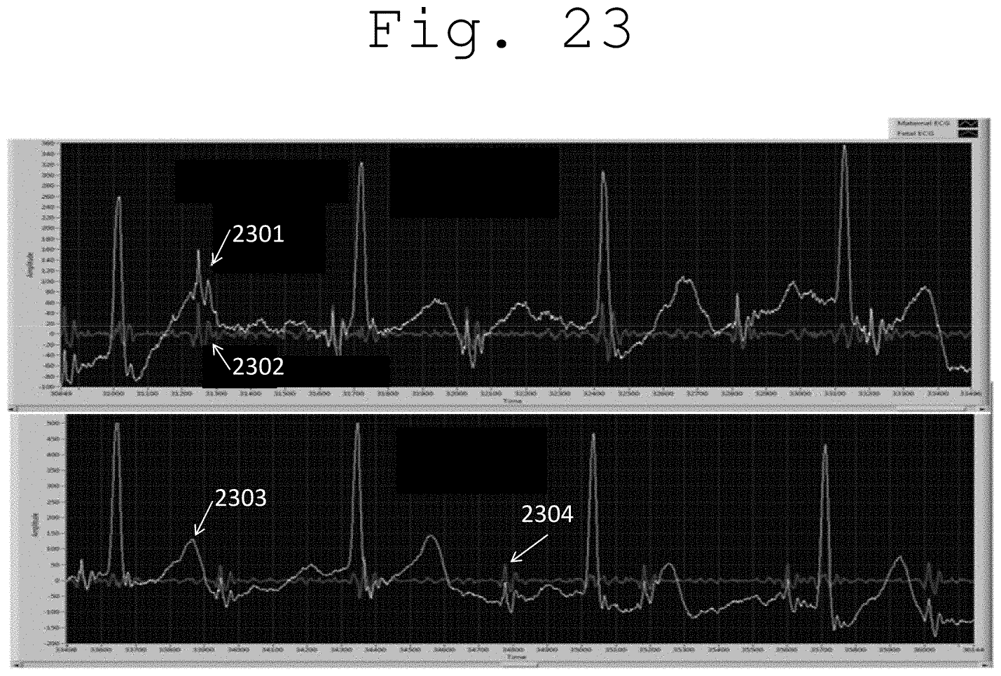

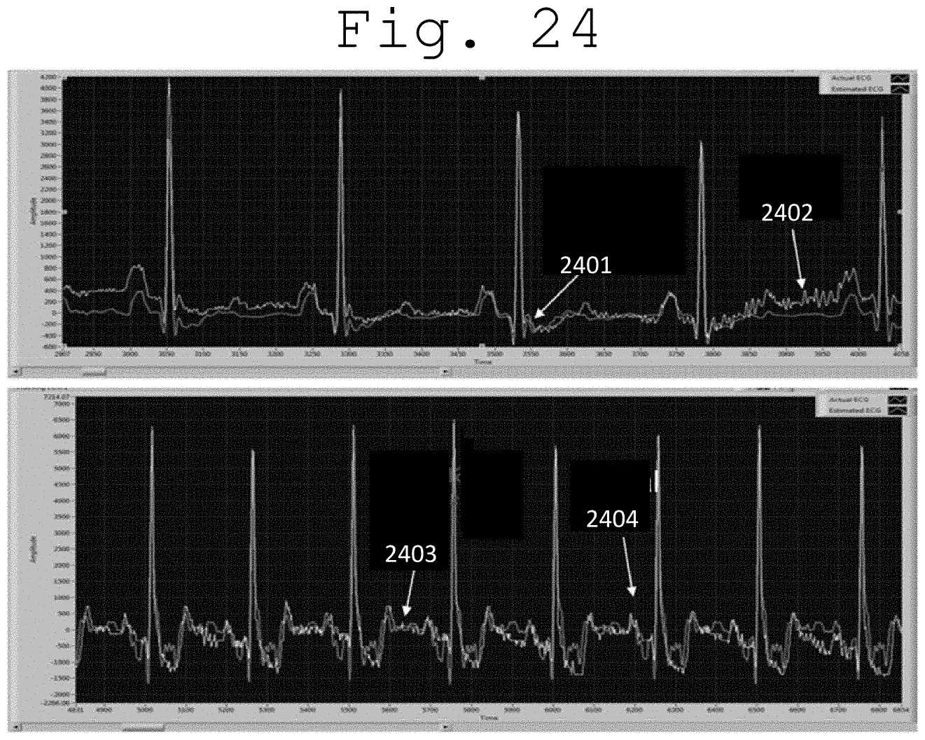

Depicted in FIG. 21, is the alternative configuration of a single patch 2101 is used to measure mixed (maternal and fetal) abdominal ECG with an extension lead wires 2104 to measure maternal ECG between maternal upper abdomen or shoulder body contacts 2102. Alternatively as depicted in FIG. 22(a), a single patch 2201 is used to measure maternal ECG using its short leads, with an extension lead wires 2204 to measure mixed (maternal and fetal) abdominal ECG between abdominal contact points 2202. In all three embodiments of FIG. 20, 21, or 22, the ECG sensing device is enabled with a processor that during a first calibration step computes the transfer function between the maternal ECG leads (model inputs) and the mixed fetal and maternal ECG (model outputs), preferably using linear or non-linear system identification methods, and more preferably using linear state space models. In a second step the identified system transfer function is used along with continuously acquired shoulder (or upper torso) maternal-only ECG signal is used as model inputs to estimate mixed abdominal maternal-fetal ECG component signal as model outputs. In a third step the estimated abdominal maternal-only ECG component is subtracted from the measured mixed fetal and maternal abdominal ECG waveform 2301 or 2303 to yield the final abdominal fetal ECG component alone 2302 or 2304, respectively. When a patch is used at either the maternal only ECG or mixed maternal and fetal ECG (abdominal) locations, the plurality of short leads ECG signals measured at one of these locations are used as model inputs or model outputs, while, substantially simultaneously, a plurality of additional ECG leads signals can be measured at the other location and used as model outputs or model inputs, respectively. The system identification can be applied using a MIMO, MISO, SIMO, or SISO configurations. The system identification transfer function between the input maternal ECG and the output mixed maternal and fetal ECG will emphasize or model maternal ECG components as primary signal and will not model or will suppress fetal ECG components as it is considered extraneous noise since it is substantially not present in the maternal-only ECG signals (shoulder or upper torso maternal ECG leads). Therefore by estimating, as an output signal from the model, the abdominal maternal ECG at mixed maternal-fetal ECG lead location, we can then easily subtract off the measured abdominal maternal ECG component using its model estimated output signal as a substitute from the mixed maternal-fetal ECG measured signal, yielding only the abdominal fetal ECG component alone as in the FIG. 23.

In another embodiment as indicated in FIG. 22(b), when a plurality of fetal babies are involved in the pregnancy, including with a twin or triplet babies, a plurality of system identification transfer functions need to be established between the maternal-only ECG (acquired with ECG sensing device 2211) and the plurality of mixed maternal and fetal ECG waveforms (acquired with ECG sensing devices 2212 and 2214). The first step will apply a system identification method to identify a transfer function, preferably linear state space model, to model the relationship between maternal-only ECG waveform (from 2211) as an input signal with each of the waveforms of the plurality of fetal ECG from each baby (from 2212 and 2214) as model output signals. In the mixed maternal-fetal abdominal ECG signals, the maternal ECG will be the primary component due to its high power, and therefore the system identification model, preferably state space linear model, will model and represent the maternal ECG as the primary signal and will de-emphasize the fetal ECG as secondary (extraneous or noise) components. The output estimated ECG signal will be that of maternal ECG at the abdominal location without fetal ECG signal. The plurality of fetal ECG components are considered noise since it is reduced or not present in the substantially maternal-only ECG leads acquired from a shoulder or upper torso area. The estimated mapped abdominal maternal ECG for each of the measured target abdominal locations will be subtracted from the measured abdominal mixed maternal-fetal ECG yielding fetal-only ECG waveform(s) at each of said target abdominal measurement locations. We preferably use a shoulder or upper torso site where measured bio-potential represents substantially maternal-only ECG, and that is used as an input into the identified system model, preferably linear state space model, and estimate the model output of abdominal maternal ECG only at an abdominal lead location. We then subtract off the model output estimate from the plurality of measured abdominal mixed (maternal and fetal) ECG component, yielding a plurality of the extracted abdominal fetal ECG components alone. The previous step is repeated for a plurality of model output(s) abdominal lead location(s), where each of the selected lead location is at a proximity to each of the fetal babies in the mother's abdomen. With every repeated step, we extract the fetal ECG from the mixed maternal-fetal ECG.

The plurality of extracted fetal ECG waveforms at each of said selected abdominal locations 2212 and 2214 can furthermore be separated from each other by using secondary steps of transfer function identification, preferably using linear or nonlinear system identification, and more preferably using linear state space models, to identify relationships across the said extracted fetal ECG waveforms from the original mixed maternal and fetal ECG. In a first step, using a first said extracted fetal ECG waveform at a first said maternal abdominal (fetal) location of 2212 as a system (model) input and a second said extracted fetal ECG waveform at a second said maternal abdominal (fetal) location of 2214 as a system (model) output. Then obtaining a relationship transfer function, preferably using a linear or nonlinear system identification model, and more preferably using linear state space model, between said first (at 2212) and said second (at 2214) extracted fetal ECG waveforms. In a subsequent second step, using continuously extracted said first fetal ECG waveform at said first maternal abdominal (fetal) location of 2212 as a system input, and using the said identified model (from previous first step) to estimate the model output representing said first fetal ECG waveform component at said second maternal abdominal (fetal) location of 2214. Then subsequently, in a third step, subtracting said estimated first fetal ECG output waveform component at said second maternal abdominal (fetal) location of 2214 from extracted said second fetal ECG at said second location of 2214 to obtain residual second fetal-only ECG waveform, effectively separating the individual babies first and second ECG waveforms from each other.

This process is repeated across the plurality of maternal abdominal locations representing different locations at proximity distance to each of the fetal babies until all fetal babies ECG waveforms are separated. The stronger waveform components (modes) that are common or shared between the inputs and outputs of the linear state space model are emphasized as primary components in the output estimates while uncommon or noise components are de-emphasized or suppressed in the output estimates as they are considered uncorrelating extraneous noise components.

In another embodiment of the transfer function definition, preferably using system identification steps, and more preferably using linear state space model, a MIMO, MISO, SIMO, or SISO configuration is used. A plurality of first extracted fetal ECG waveform(s) at a plurality of first maternal abdominal (fetal) location(s) are used as system (model) input(s). Similarly, a plurality of second extracted fetal ECG waveform(s) at a plurality of second maternal abdominal (fetal) location(s) are used as system (model) output(s).

In all of the above discussion, if abdominal ECG can not be obtained due to a surgical procedure, such as a C-section, then ECG can be measured from a body side (lateral) location or dorsal location for each of the distinct bio-potential lead locations which will yield equivalent results to the use of abdominal ECG locations.

In yet another embodiment, the target fetal ECG is extracted from the mixed maternal and fetal ECG as a first step yielding the target fetal ECG as a desired model output, and maternal plus other fetal ECG components as modeled as undesired noise components. A mixed fetal plus maternal ECG is used as input to a model being identified with a transfer function, preferably using a system identification method, and more preferably using a linear state space model, with the output being an estimated maternal-only ECG at a shoulder or upper torso location. This will yield an estimated maternal-fetal ECG at the shoulder as desired output, which when subtracting measured shoulder maternal-only ECG, yields the fetal ECG as a (noise or undesired) residual ECG waveform.

If more than one fetal baby is present, this process is repeated to yield another residual fetal ECG for each of the remaining babies. A first residual fetal ECG is then selected and used as an input into another transfer function being identified, preferably using system identification methods, and more preferably using linear state space model, with the output being a second (another) target residual fetal ECG. The estimated output will contain the extracted second estimated output fetal ECG with the first input fetal ECG represented as noise or undesired signal component. The undesired component can be extracted by repeating this step with using the second fetal ECG as input and first fetal ECG as output, or by simply subtracting said extracted estimated second ECG component from measured ECG at second location, or by simply subtracting an extracted estimated first ECG component from measured fetal ECG at first fetal location. This process is repeated until all fetal ECG components for each of the babies are extracted.

In yet another preferred embodiment, in addition to the previously said use of single input single output (SISO) models or transfer functions, a more efficient processing would include the use of a multiple input multiple output (MIMO) or multiple input single output (MISO) or multiple output single input (SIMO) transfer functions, preferably system identification methods, and more preferably linear state space models in the estimation of output(s) as either mixed maternal-fetal, extracted maternal, and/or fetal ECG using input(s) as either mixed maternal-fetal, extracted maternal, and/or fetal ECG waveform.

EEG, EOG, EMG, or Neurologic Source Separation, and Undesired Signal or Artifacts Removal:

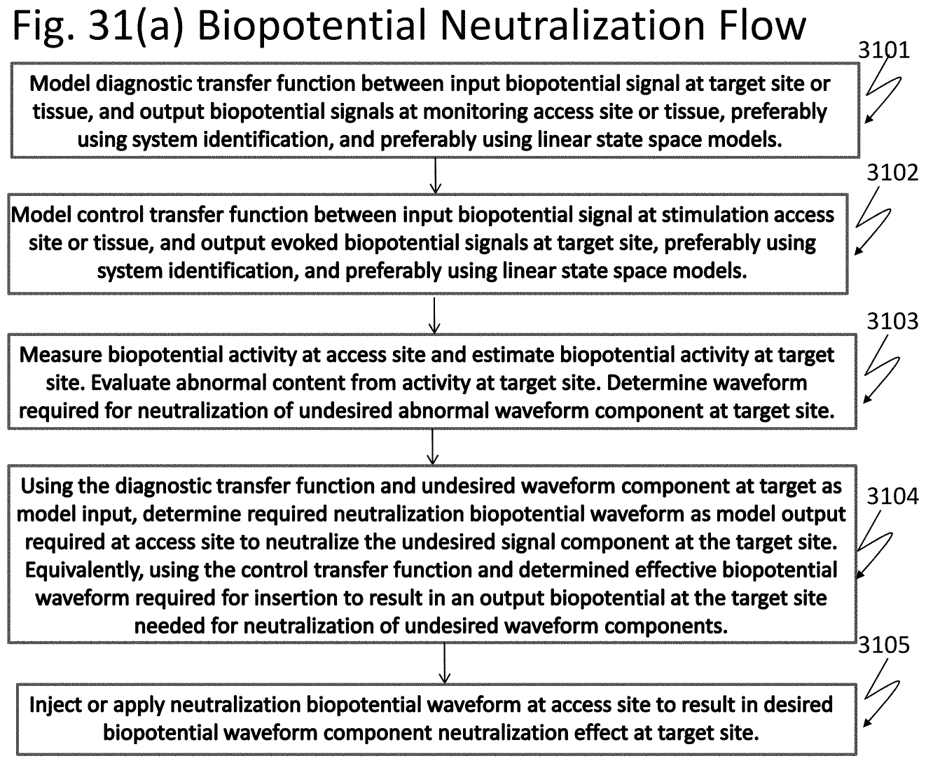

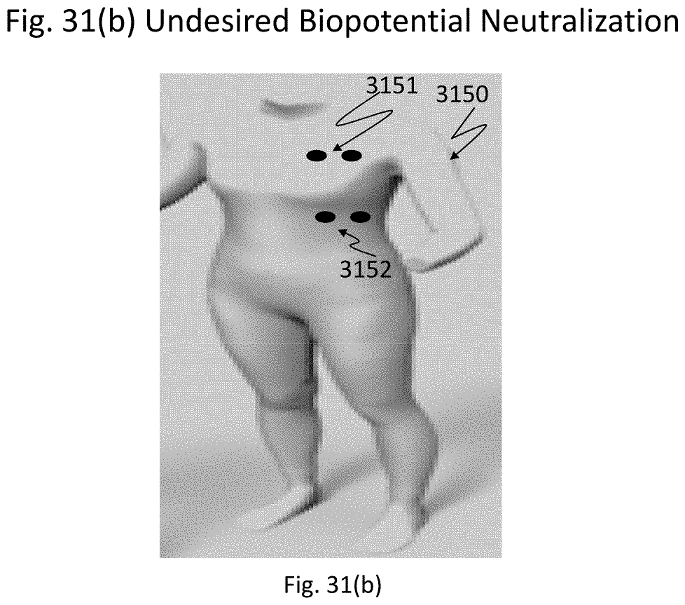

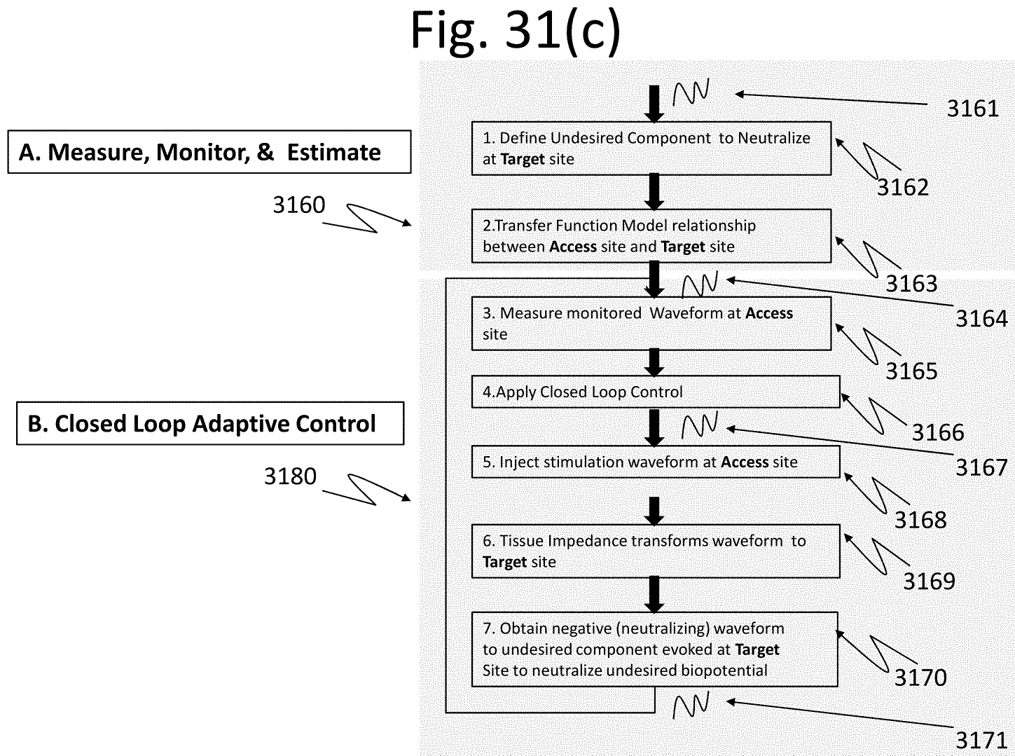

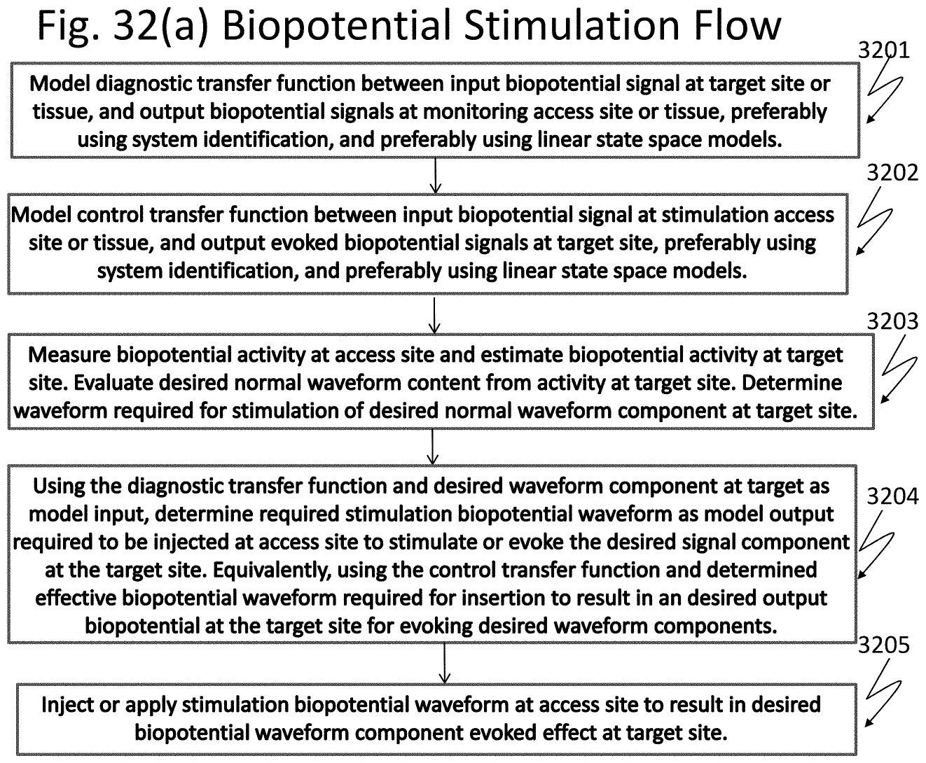

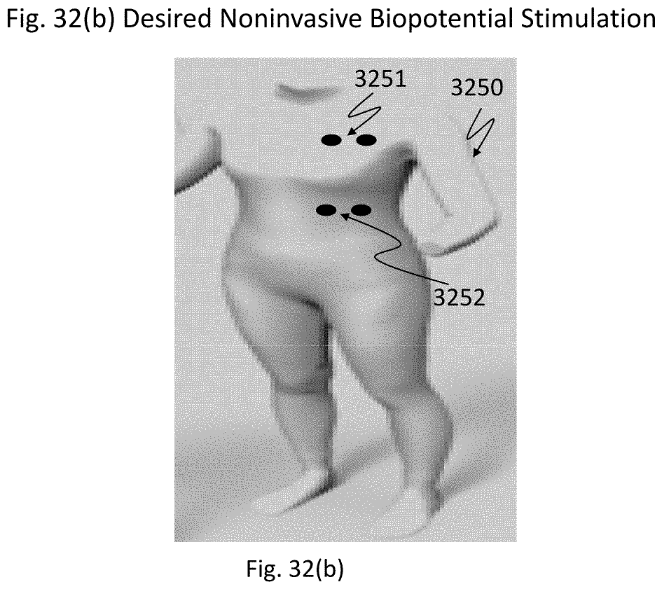

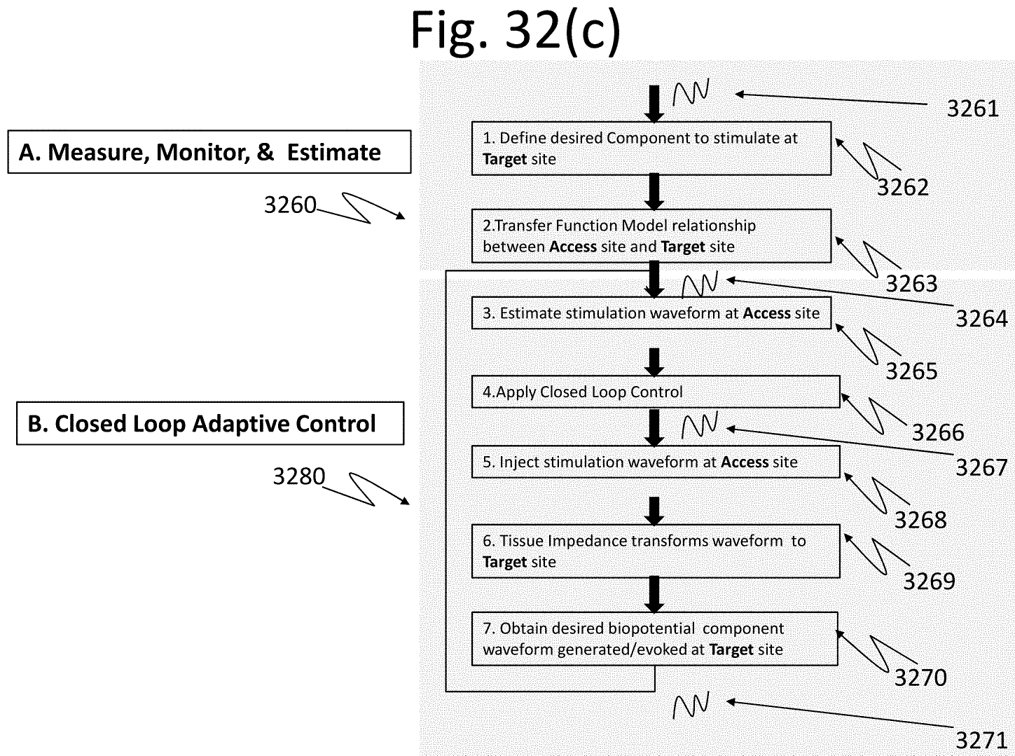

FIGS. 31(a), 31(b), 32(a) and 32(b) relate to the following discussion. The above discussion on maternal and fetal ECG (electrocardiogram) separation can be extended to other applications in electrophysiologic signals, including EGM, EEG, EMG, and EOG, and generalized to interfering biopotential waveforms extraction or signal separation, as well as removal or filtering of in-band noise sources or artifacts or undesired biopotentials interfering with desired biopotential signals. For example, for applications separating EMG (electromyogram) and EEG (electroencephalogram) waveform components, separating ECG and EEG waveforms, separating EOG (electrooculogram) and EMG waveforms, separating EOG and EEG waveforms, separating left EOG from right EOG waveforms, separating components or sources of EEG from other components or sources of EEG waveforms, or separating EGM (cardiac electrogram) components or sources from other EGM components or sources, or separating EMG components or sources from ECG components or sources, or separating EMG components or sources from other EMG components or sources, or separating neurological biopotential components from other neurological biopotential components, or separating evoked biopotential components or sources from other evoked biopotential components or sources, or separating undesired motion artifacts components from any of EEG or ECG or EMG or EOG or EGM or neurological or evoked biopotential waveforms. Whereby a first component or source is used as input(s) and a second component or source is used as output(s) into one or more transfer functions, preferably using linear or non-linear system identification methods, and more preferably using a linear state space model, in either a SISO, MISO, SIMO, or MIMO configurations. Same fundamental concepts apply, as in our previous discussion on extracting fetal ECG from mixed maternal-fetal ECG, with the only difference being is that the input and output biopotential signals may contain different frequency spectral content as compared to other type of biopotential signals. Generally, for different types of signal sources, the structure of relationship transfer functions for the system and methods are the same, except the model order may vary, and the types of inputs and outputs waveforms may differ based on the application need for waveforms to be separated. This source separation method is effective even when the two signals are overlapping in the same frequency spectral bands in their content. It is therefore advantageous over traditional low pass, band pass, or high pass filtering methods, and is also advantageous over adaptive filtering methods because it does not require continuous adaptation during the separation of the sources in real time, rather the adaptation of the model is applied only during the model identification step.

The above method for signal source separation can be more generalized and expanded to include any type of two waveforms types desired to be separated including voice or speech, or measured sensory signals whether, acoustic, optical, electromagnetic, electrical, magnetic, or mechanical energy signals, and whether the source is biological, chemical, or physical in nature. The previously described method of signal source separation of biopotential waveforms can also be applied to separate waveforms from two separate sources that are overlapping in frequency on top of each other. For example the method can be applied for separation of heart sounds using an array of acoustic sensors, or monitoring of respiration breathing sounds and their separation from other sounds including cardiac sounds, or speech, or more generally laryngeal sounds including snoring sounds. Similarly, more generally in separation of ambient or undesired noises from primary desired signal. Similarly, in separation of desired speech or sound from a plurality of undesired speech or sound sources, including snoring source separation, Additionally, other applications are in separation of sources of cardiac arrhythmia in ECG or cardiac fibrillation as useful in identifying its source as originating from Atrial or Ventricular region or tissue, or as originating from left or right cardiac chamber source, or as originating from a specific section or tissue source within a particular cardiac chamber. Similarly, another application is in the separation of sources of seizure in the brain as originating from a particular tissue section or region in the brain. More generally, system identification can be applied for separation of sources of interference in a waveform.

In each of the above examples at least two sensors are used whereas the first sensor is measuring a desired primary biopotential signal source and an undesired (interfering) secondary biopotential signal source, and whereas the at least second sensor is measuring the said first sensor's primary biopotential signal source as an undesired secondary biopotential signal source and the said first sensor's undesired biopotential signal source as now the second sensor's desired primary biopotential signal source. The at least two signals from the at least two sensors are now used as either input(s) or output(s) to linear or nonlinear system identification models, preferably linear state space models, including SISO, MISO, SIMO, or MIMO structures or configurations.

To separate out the primary and secondary modes or biopotential signal sources in same method as described above for separating maternal and fetal ECG as in FIGS. 20, 21, 22(a), 22(b), and 23. The system identification method results in de-emphasizing, filtering, or suppression of secondary modes or noise waveform components and emphasis or retention of the primary modes or desired waveform components in the estimation process output. This biopotential signal source separation using system identification process, preferably linear state space models, can be re-iterated multiple times to further reduce the power or content of undesired biopotential signal sources and enhance the biopotential signal source separation process performance. In each iteration, we use the previous separated output of desired and undesired biopotential signal components as model inputs and outputs to the next biopotential signal source separation step by system identification.

In another embodiment, the system identification methods identifying a transfer function, preferably using linear state space model, can also be used to identify the transfer function modeling the relationship between inputs represented by at least one of the standard ECG lead(s), that are connected and measured, to outputs, represented by the other standard ECG leads that are either disconnected, or connected but noisy with undesired artifacts, signal noise (including power line frequency 60 Hz or 50 Hz), experiencing baseline shifts due to pressure on the electrodes or peeling off of the electrode, or due to motion artifacts during patient activity. In a first step, when the input ECG standard lead(s) (for example, at least one of lead I, II, III, V1, V2, V3, V4, V5, V6) and the output ECG standard lead(s) (for example at least one of residual leads I, II, III, V1, V2, V3, V4, V5, V6) are acquired with minimal noise, we use system identification methods, preferably state space linear systems, to identify the system transfer function between input(s) and output(s), which yields the transfer function model structure and its coefficients. In a second step, we use the identified transfer function relationship from the previous step with continuously acquired model input(s) waveform(s) to estimate model output(s) waveform(s). When the ECG monitoring system experiences a disconnected, noisy, artifact related, wandering baseline, or motion related lead channel, then the ECG monitoring system replaces the troubled lead channel measured waveform with the estimated waveform from the model outputs, which is generated using the previously identified model and its related input waveforms currently acquired. This invention advantageously reduces disconnected leads alarms, and lost or noisy data. In a preferred embodiment, in addition to SISO models, either MIMO or SIMO or MISO model structures can be used for identified transfer function relationships.

This recalibration procedure facilitates to identify systems that are fairly similar yet slightly optimized to meet better quality estimation requirements of the output standard “long-leads”. Furthermore, it is possible to constrain the estimation of the system parameters identified within a reasonable range from the previously identified system parameters (with upper and/or lower boundary limits), in order to avoid divergence to an unacceptable state of the identified system.

In summary, we described a biopotential measurement and separation method for measurement of electrical activities from two or more bio-potential sources in a subject's body, comprising:

providing at least one multi-contact bio-potential electrode assembly adapted for attachment to the subject's body, said electrode assembly being formed of an electronic layer and an electrode layer, said electrode layer having a plurality of contact points for engagement with the surface of the subject's body;

measuring, using the at least one multi-contact bio-potential electrode assembly, first short-lead bio-potential input signal(s) in response to electrical activity from a first bio-potential source in the subject's body; measuring, using the at least one multi-contact bio-potential electrode assembly, first long-lead bio-potential input signal(s) in response to electrical activity from a second bio-potential source in the subject's body;

producing, using a processing unit, a transfer function using the first measured short-lead bio-potential input signal(s) and first measured long lead output signals; using said transfer function to compute estimated long-lead bio-potential output signal(s) based on second measured short-lead bio-potential input signal(s) from said plurality of contact points; and subtracting said estimated long-lead bio-potential output signal(s) from second measured long-lead bio-potential output signal(s), thereby identifying residual signal(s) component(s) present in the output signals, said residual signal(s) component(s) substantially representing electrical activity from one or more sources other than the first bio-potential source. In an embodiment, at least one biopotential short lead(s) and/or long lead(s) of a first electrode assembly provides the calibration biopotential lead(s) output signal(s) to at least another short lead(s) and/or long lead(s) input signal(s) of either the first or a second electrode assembly. In an embodiment, localization of said residual signal(s) electrical biopotential source within tissue is determined by at least two successive iterations of residual signal(s) computation. In an embodiment, output bio-potential signal(s) represents mixed maternal-fetal ECG, and input bio-potential signal(s) represents maternal-only ECG, for monitoring heart activity. In an embodiment, output bio-potential signal(s) represents mixed electroencephalogram (EEG) and electromyogram (EMG), and input biopotential signal(s) represents only EEG, for monitoring brain activity. In an embodiment, output biopotential signal(s) represents mixed electrocardiogram (ECG) and electroencephalogram (EEG), and input biopotential signal(s) represents only EEG, for monitoring brain activity. In an embodiment, output biopotential signal(s) represents mixed electrocardiogram (ECG) and electromyogram (EMG), and input biopotential signal(s) represents only ECG, for monitoring heart activity. In an embodiment, output biopotential signal(s) represents mixed electroencephalogram (EEG) and electrooculogram (EOG), and input biopotential signal(s) represents only EEG, for monitoring brain activity. In an embodiment, output biopotential signal(s) represents mixed normal electrocardiogram (ECG) and abnormal ECG, and input biopotential signal(s) represents only normal electrocardiogram (ECG), for monitoring heart activity. In an embodiment, said residual signal(s) represents abnormal cardiac electrocardiogram or arrhythmia of heart activity. In an embodiment, output biopotential signal(s) represents mixed normal cardiac electrograms (EGM) and abnormal EGM, and input biopotential signal(s) represents only normal EGM, for monitoring heart muscle activity. In an embodiment, said residual signal(s) represent abnormal cardiac electrogram (EGM) of heart activity. In an embodiment, output biopotential signal(s) represents mixed normal electroencephalogram (EEG) and abnormal EEG, and input biopotential signal(s) represents only normal EEG, for monitoring brain activity. In an embodiment, said residual signal(s) represents abnormal brain encephalogram (EEG) of brain activity. In an embodiment, output biopotential signal(s) represents mixed normal electromyogram (EMG), and abnormal EMG, and input biopotential signal(s) represents only normal EMG, for monitoring muscle activity. In an embodiment, said residual signal(s) represent abnormal electromyogram (EMG), of muscle activity. In an embodiment, output biopotential signal(s) represents mixed normal electroneurogram (ENG) and abnormal ENG, and input biopotential signal(s) represents only normal ENG, for monitoring nerve activity. In an embodiment, said residual signal(s) represent abnormal electroneurogram (ENG) of nerve activity. In an embodiment, said residual signal(s) represents an evoked potential (EVP) of tissue activity. In an embodiment for continuous real-time calibration and separation of biopotentials, said first short-lead bio-potential input signal(s) and said second short-lead bio-potential input signal(s) are the same; and said first long-lead bio-potential output signal(s) and said second long-lead bio-potential output signal(s) are the same. In an embodiment, said residual signal(s) from said one or more sources represent stimulated evoked potentials provided at said plurality of contact points for said long-lead biopotential contacts. In an embodiment, said residual signal(s) from said one or more sources are substantially neutralized by providing an evoked potential representing the inverse of said residual signal(s) at said plurality of contact points for said long-lead biopotential contacts.

The leadless wireless ECG measurement system and method of the present invention has the following advantages over the prior art as follows:

(1) it provides clinical equivalence to standard lead ECG measurements;
(2) it utilizes a personalized per patient calibration so as to ensure accuracy;
(3) it reduces cost to the patient and health care giver, and reduces time for care giver to sort out wires;
(4) it reduces substantially motion artifacts due to elimination of leads (especially for Holter monitoring and stress monitors);
(5) it reduces opportunities for infection due to wires exposure to body fluids;
(6) it increases comfort for patients by elimination of wires;
(7) it eliminates leads-off alarms due to tugged wires;
(8) it eliminates wrong lead connection attachment;
(9) it produces a standard multiple-lead ECG with a single wireless patch application, thereby reducing number of leads required to produce a fully diagnostic 12-lead, 5-lead, or 3-lead ECG; and
(10) it produces potential expansion markets which include Stress ECG monitoring, Holter monitoring, and continuous surface ECG monitoring via implanted Pacemakers or internal and external defibrillators.

From the foregoing detailed description, it should be clearly understood that the leadless ECG measurement system is composed of at least one multi-contact electrode assembly attached to the patient body and a remote monitor in communication with the at least one multi-contact electrode assembly. The electrode assembly includes an electrode layer with a plurality of contacts for contacting the skin's surface on the patient, preferably made of flexible membrane polymer, for comfortable fit to the skin's curvature, and an electronic patch layer disposed on top of the electrode layer, to which the electronic components are mounted.

This system of the present invention can also be used as just an add-on or supplementary to the standard measured 12-lead ECG system, as an enhancement providing substitute lead estimation to measured leads when needed. For example, in case we loose a lead's contacts (e.g. electrode peals off) or a lead experiences motion or noise artifacts in the signal, then we can compensate for the measurement noise error (suppressing its presence) or completely replace the faulty measured standard lead with a calculated estimate of that standard lead. The estimates can be derived by using data from other good standard leads as inputs and identified models relating these inputs to the faulty output signal now being estimated. This strategy enhances the acquisition of standard 12-lead ECG by making it more robust to noise factors. In other words, leadless ECG algorithms and methods can be used as an auxiliary to existing ECG platforms and not just as a stand-alone platform.

Model estimated ECG signals can be further processed with algorithms for detection, recognition, classification, and alerting of abnormal ECG patterns including Arrhythmia ECG, Bradycardia ECG, Tachycardia ECG, bundle branch block, cardiomyopathy, Dysrhythmia, Atrial or Ventricular fibrillation, Alternan ECG, ischemia or stenosis of arteries, congestive heart failure, myocardial infarction, etc. can be done to enable such active detection of abnormalities and alarming against such events. The electronic patches can optionally have an alarm speaker that delivers the alarm, or can transmit the alarm status to the wireless communication monitor if connected for further annunciating the alarm, or email messaging the alarm status and information which may include a sample of the abnormal digital ECG waveform data for immediate viewing of the alarmed abnormal ECG segment, or telephoning an emergency contact phone number with the alarm message, or text messaging the alarm message to a text message receiving phone number. Alternatively the alarm events can be stored in memory in case there was no communication link to the remote monitor. Cross-referencing of all event information across the electronic patches can also occur to match events detected independently on each of the electronic patches, if applicable. A table of such cross-referencing events can be generated at the monitor, or the information can be equally indicated on a graphical representation of each of the channels/patches acquired or calculated channels. The monitor can also display a graphical approximate physical location of those acquired or calculated bio-potential channels represented relative to a human body diagram.

The discussion of the present invention has thus far focused mainly on electrocardiogram (ECG) waveforms processing; however, it should be clearly understood that ECG is only one of several bioelectric physiologic signals of interest that are measured on the surface of the skin. Other signals of interest include electroencephalography (EEG), electromyography (EMG), electrooculography (EOG), electrogram (EGM), neurograms (NGM), and evoked potentials.

Cardiac Electrogram (EGM) to Surface ECG Mapping:

The discussion below is in reference to FIG. 24. Implanted pacemakers typically measure Electrograms (EGM) directly on the cardiac muscle (either internally or externally to the cardiac muscle). A relationship system transfer function, preferably using system identification methods, and more preferably using linear state space models, in either SIS, SIMO, MISO, or MIMO structure and of any model order (preferably order 1 to 1000), can be identified mapping as model input(s) EGM waveform(s) into model output(s) of surface ECG waveform(s), preferably in standard 12-lead ECG configuration of leads I, II, III, V1, V2, V3, V4, V5, V6 and augmented leads aVF, aVR, aVL. This method provides continuous estimated standard surface ECG monitoring by the pacemaker and simplifying the interpretation of EGM by clinicians, regardless where the placement is for the physical pacing leads or electrodes on the cardiac muscle was. The surface ECG view of cardiac muscle EGM offers a continuous diagnostic interpretation that is more intuitive to understand in its details and content versus an otherwise much more difficult to interpret EGM signals. Depending on where the affected cardiac tissue is, and where the placement of the measuring leads contacts are, an EGM may offer different information for different patients, however, a surface ECG is a more common standard perspective for cardiac electric activity interpretation, as in the FIG. 24. Depicted are two examples of actual measured surface ECG signals, 2402 or 2404, being presented as model outputs to a linear state space models with inputs being cardiac electrogram (EGM) signal source on the cardiac muscle. After model identification of its structure and coefficients, said identified models are used with the input cardiac EGM signals to produces as output the estimated surface ECG signals 2401 or 2403, respectively. Said models are relating as input cardiac electrogram (EGM) and output surface ECG, which is advantageously useful when we implanting an internal pacemaker or defibrillator with leads on the cardiac muscle to apply a first calibration step to identify the model structure and coefficients, preferably linear state space model, linking the measured EGM inputs with standard surface ECG outputs. In a second step, said identified model is used to produce surface ECG signals as estimated output, from the directly measured cardiac EGM signals, as model inputs. Estimated surface ECG output signals are advantageously much easier to interpret than the source EGM signal for presence of abnormalities or arrhythmias and their location or direction of propagation.

In another alternative embodiment, evoked bio-potentials can also be used in identifying the system relating the evoked input(s) and the measured output bio-potential response(s). For example, the evoked input can include an optical light stimulation of varying intensity at a plurality of frequencies and amplitudes, or an auditory pulse or waveform at a varying plurality of frequencies and amplitudes, or an electric stimulation signal injected onto the skin or muscle typically of relatively higher voltage and low current characteristics (such as for EMG stimulation) also at a plurality of frequencies and amplitudes. In any form, the evoking signals can act as inputs while measured evoked bio-potentials can act as outputs, and the plurality of inputs and plurality of outputs are then presented to the system identification tools, preferably linear state space models, for model coefficients and structure identification that provides best correlation between estimated output signals and actual output signals. This can be advantageously used to assess effects of factors influencing the physical system being modeled, including disease state or medication dosage, on the evoked potential signal strength and delay in timing. Applications include auditory testing and neural conduction testing and evaluation in general.

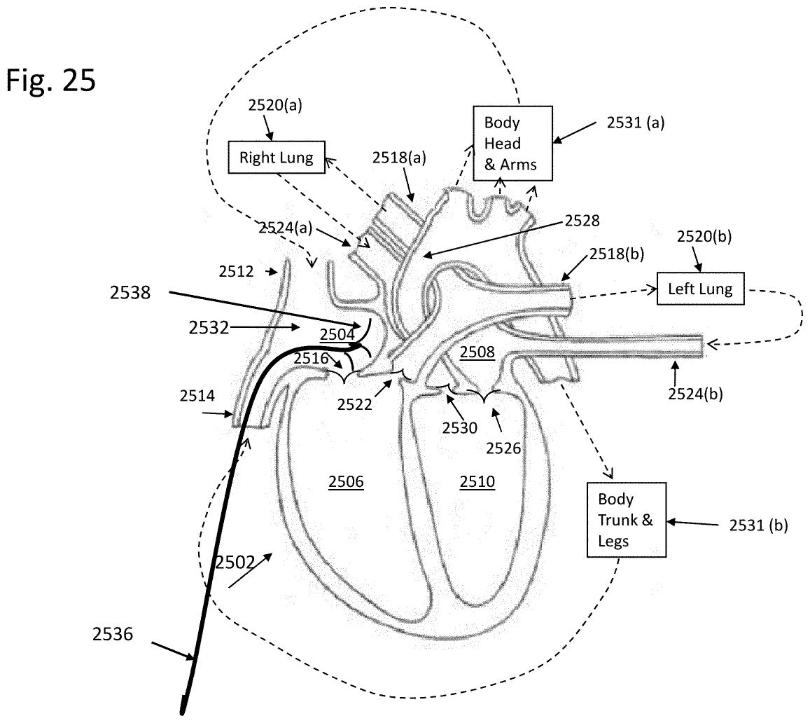

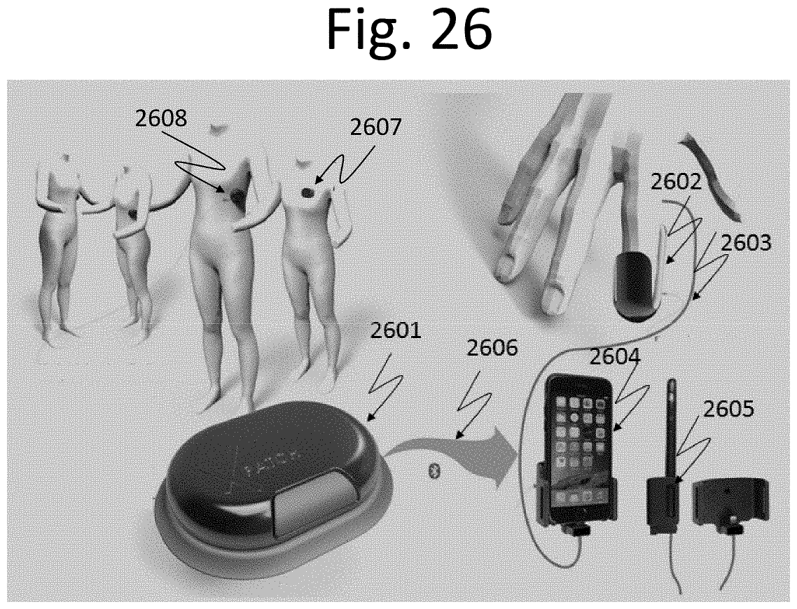

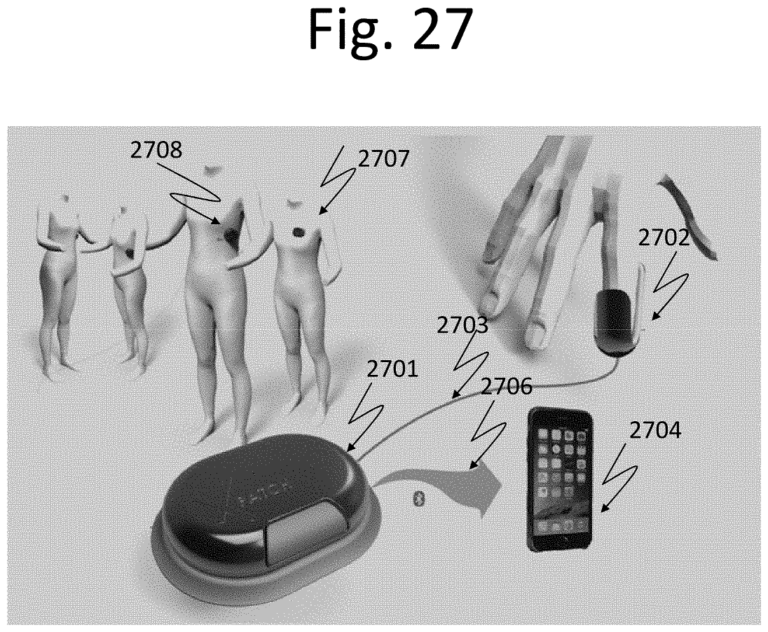

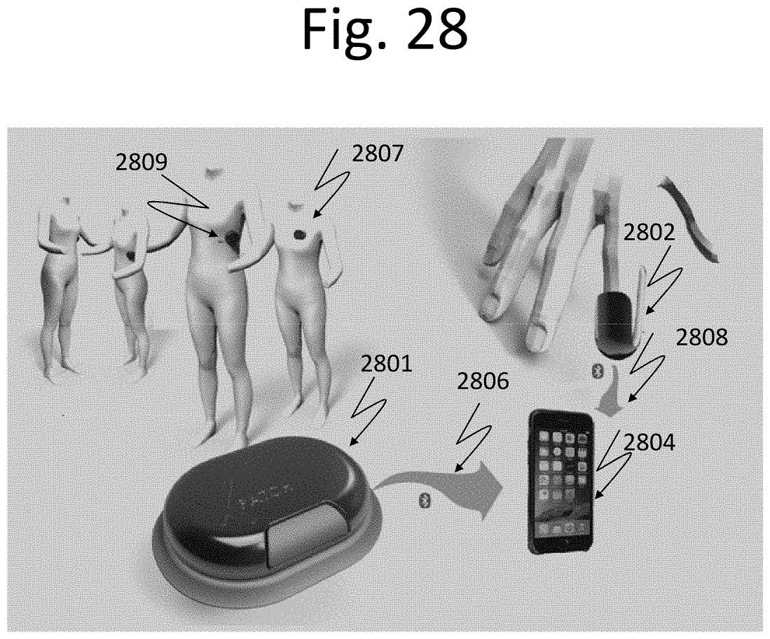

With attention directed to FIG. 25 of the drawings, there is illustrated a diagram of the cardio-vascular system and its cycle. The heart 2502 consists of four chambers; namely, the right atria 2504, the right ventricle 2506, the left atria 2508 and the left ventricle 2510. The right atria 2504 receives carbon dioxide-filled blood which is returning from the body through the superior vena cava 2512 and the inferior vena cava 2514 where they join in the central venous. The right ventricle 2506 receives the blood from the right atria 2504 via a tricuspid valve 2516 located between the two chambers. The right ventricle 2506 pumps the blood via a pulmonary valve 2522 into the pulmonary artery 2518 which branches into the right pulmonary artery 2518(a) for carrying the blood to the right lung 2520(a), and the left pulmonary artery 2518(b) for carrying the blood to the left lung 2520(b). After receiving oxygen in the lungs 2520(a) and 2520(b), the oxygen-filled blood is returned to the left atria 2508 of the heart 2502 via the right pulmonary vein 2524(a) and the left pulmonary vein 2524(b). The blood is then passed into the left ventricle 2510 via mitral valve 2526. Next, the left ventricle 2510 pumps the blood via the aortic valve 2530 into the aorta 2528 which branches into the ascending aorta for delivery throughout the upper body 2531(a) heads and arms, and the descending aorta 2531(b) for delivery throughout the lower body including the trunk and legs via the network of arteries, capillaries, and finally returned to the heart 2502 via the superior vena cava 2512 and the inferior vena cava 2514, which both merge into the center venous 2532. Both the right atria 2504 and the left atria 2508 pump simultaneously, and both the right ventricle 2506 and the left ventricle 2510 pump simultaneously. In every pumping cycle, each chamber undergoes an expansion cycle called diastole followed by a contraction cycle called systole. On the ECG waveform, the Atrial diastolic phase extends between the S in the QRS complex until the start of the following P wave, while atrial systole is between the start of the P wave and extends through the following S in the QRS complex. Similarly, the Ventricular systolic phase is between the R in QRS-complex and the following end of the T-wave, while ventricular diastolic phase is between end of the T-wave and the following R in the QRS-complex. The atrial systolic phase occurs mostly during ventricular diastolic phase with a small fractional overlap to ventricular systole, while the atrial diastolic phase starts with the ventricular systolic phase but also extends and overlaps a substantial portion of the ventricular diastolic phase. Atrial diastolic phase has a longer duty cycle as compared to Atrial systolic phase. A probe catheter, 2536, is advanced into a cardiac chamber, preferably through a vascular vessel, which is used to sense and measure electric biopotential activity directly on the cardiac muscle. A plurality of sensing electrodes wires, 2538, with electrical conductive contact points and preferably mechanically supportive protective and electrically insulating sheeting around each wire are advanced to touch the cardiac muscle tissue and record electrogram activity directly from the cardiac muscle.

Source Separation and Localization and Treatment:

FIGS. 31 (a), 31 (b), 31 (c), 32 (a), 32(b), and 32 (c) relate to the following discussion. In another embodiment, the source separation algorithm discussed herein provides source localization for cardiac tissue to identify sources of abnormal biopotential waveform generation or effects, including ECG arrhythmia or fibrillation. The same discussion provided herein applies for all other biopotential signal types, including, electroencephalography (EEG), electromyography (EMG), neural recordings, electrooculagraphy (EOG), and evoked potentials type signals. Source separation for the EEG signals, whether measured on the skin surface or directly internally on brain tissue can help identify sources for seizures and tremors in the brain tissue. The discussion of the present invention on leadless measurement implementation, source separation, and therapeutic neutralization of abnormal signals and their impact, is intended to extend equivalently to the brain as an organ, replacing cardiac muscle tissue with brain tissue. Source separation can be performed on measurements of naturally stimulated biopotentials or as a result of (externally) evoked biopotentials. Evoked biopotentials are external stimulation of the tissue with electrical signals at an appropriate frequency range for stimulating the target tissue and at a user selected periodicity, including pacing. Simple conductive tissue does not add electric current or new waveform frequency components to the signal content of the stimulus signal, however tissue that provides an electric source adds an electric current frequency components to the signal content, resulting in an arrhythmia or a tremor or fibrillation in a muscle. Tissue can also act in impacting electrical pathway conduction properties, including signal blocking, rerouting, or delaying. We are not only assessing the ability of muscle tissue to conduct electric current as it normally is expected to, but also its ability to either block (consume or delay or redirect current) or add (i.e. stimulate with additional current) current to the pacing traveling wave. Typically, electric current that is generated by the natural cardiac pacemaker is consumed with every cycle, so that there is no accumulation of current from one cycle to the next. Typically, the heart achieves an electrical silence between one pulse (cycle) and the next. When the muscle tissue conducts the electric current, we expect to see all of the frequency components from the input signals into the state-space model mapped (via the transfer function relationship) to the output signal components of the state-space model, unless they were suppressed by zeros of the transfer function. In other words, the adaptively identified system using linear or nonlinear system identification methods, preferably linear state-space models, should converge on a solution that represents a transfer function that effectively models the relationship between the input lead biopotential signal and the output lead biopotential signal, and estimates the output signal from the input signal, so that the difference between the estimated output signal and the actual output signal has minimal residual estimation error. However, when the muscle tissue exhibits abnormal symptoms including block (reroute), delay, or stimulate with additional signals, The results of the subtraction of the model's estimated output signal from the actual measured output signal is indicated by one of the following observations:

1—Block or Reroute Signals: Blocking or rerouting signals is a characteristic of the tissue conductivity. The system transfer function estimated output will have suppressed signal components relative to input response if the suppression is part of the conductive pathway tissue being modeled in this relationship. If the suppression if not part of the conductive pathway itself but is occurring due to the interaction of the output measured lead with extraneous sources resulting in the neutralization or removal of signal components that otherwise should be in the output signal, then the subtraction of the estimated output from the actual measured output lead signal will result in negative values in the resulting residual signal or waveform.
2—Stimulate or Add Signals: The system transfer function actual output will contain additional components on top of the actual estimated output signal (i.e. system response) to the input stimulus signal, the subtraction of the estimate output from the actual output will result in positive values in the resulting residual signal or waveform.
3—Delay Signals: Delaying signals is a characteristic of the tissue performance. The system transfer function estimated output may not converge on a solution with a reasonable minimal residual error, especially when using linear transfer functions, preferably state-space linear models, as these models do not perform well in representing delay relationships between inputs and outputs in general, such as when the delay order (i.e. equivalent to number of sampled steps) is larger than the model order (i.e. number of states). Model order can be increased to estimate the delay order until convergence of estimated output and actual output occurs.

FIGS. 32(a), 32(b), 32(c) relate to the following discussion. We need a measurement and modeling step 3201, where in the biopotential at a patient's 3250 target tissue site 3252 (typically not easily accessible such as on the cardiac muscle) (presented as either model input or output) is related using a transfer function 3263 to the biopotential at an access tissue site 3251 (presented as either model output or input, respectively) (typically easily accessible such as on the surface of the skin or subdermal). The transfer function relationship is preferably identified using a system identification method. The identified system relationship, preferably state-space model, will capture and represent the tissue properties of the tissue relating or connecting the input signal and the output signal. Similarly in a stimulation step 3202, a model is identified describing the transfer function relationship between an access tissue site signal (typically easily accessible such as a contact on the patient's skin or subdermal) is stimulated with an injected stimulation signal (presented as either model input or output), and generating an evoked biopotential at a target tissue site (presented as model output or input, respectively). Wherein the access tissue sites and/or the target tissue sites in both the measurement step 3201 and the stimulation step 3202 can be either the same sites or different sites. In 3203, an evaluation of desired normal waveform content in the target tissue site is determined 3262, and in 3204 the required biopotential waveform that is needed to be injected (stimulated) at access tissue site to generate the desired biopotential waveform at the target tissue site (evoked) is created 3265, and then injected in step 3205. Due to dynamic nature of biopotentials, a model free closed loop controller 3266 provides a controller output signal 3267 applied to minimize the residual error difference signal between the measured desired biopotential 3271 and the defined desired biopotential 3261.

The system model represents a dynamic relationship between the input and output signals depicting the sources of waveform morphological features that are indicated in the output signal as being a result of the direct input signal features or due to tissue property or if they are due to presence of external sources (undesired, noisy, or interfering signal source) that are contributing to affecting the output signal waveform morphology, for example, an external source that is stimulating or generating the electric current, such signals are herein termed a biopotential Source signal. By comparison, a blocked or filtered input signal component that does not appear in the output signal will be captured or represented by the model's transfer function as having a zeros at that frequency that is suppressing these filtered or suppressed components. By comparison, a pole of the transfer function at a particular frequency will amplify or strengthen the representation of that waveform frequency component in the output signal waveform. For state-space functions, the state variables represent the unforced dynamics relationship between the inputs and outputs of the model. If an input signal frequency component is not mapped to an output signal frequency component while there is no zeros in the transfer function that is explicitly suppressing these frequency components, it can be suppressed in the output biopotential signal as a result of external biopotential signal sources that are interfering with the output and effectively neutralizing, or cancelling out and removing that frequency component. In that case, the subtraction of the estimated output signal from the actual output signal will result in a negative value for that output signal component being suppressed by an external interfering source. Such a signal is herein termed a biopotential Sink signal. A Delayed signal, depending on the delay steps relative to the model order (number of states), may be represented by the model if model order is larger than the delay steps, given the sampling frequency, or may otherwise not be represented causing nonconvergence of the model on a solution for the output signal. Delay can be estimated using a number of standard methods including cross-correlation between the input and output model signals. A delay characteristic can be variable for a tissue region of interest as well. Such delays estimations are herein termed a Delay signal.

Electrical biopotential Source or biopotential Sink localization can be successfully be achieved using successive steps of source separation process looking for added signal components with tissue that adds biopotential signals (stimulating electrical current), neutralize biopotentials, or delay biopotentials. Using a series of sequential application steps involving the process of measuring of biopotential sources, adaptive modeling of the relationships between these sources, identifying source versus sink tissue characteristics and relationships to surrounding tissue, relating these identified sources and sinks to an anatomical spatial location in three dimensions creating a map for conductive pathways and their properties, and associating these electrical sources and sinks maps to physical cardiac anatomy using imaging methods that allow registration, orientation, and co-location of electrical map with the physical map. Similarly, interfering conduction pathways and biopotentials can be identified using electrical source localization using successive source separation process application as stated above, except we are looking for any missing (subtracted or removed) signal components instead of added signal components.

This source separation process can be conducted on the cardiac tissue at any desired spatial resolution in the direction of interest, or region of interest, and at any orientation desired by the clinician. The measurement lead contact points define a grid of points that can be defined by a uniform pattern, or, preferably, more flexibly a non-uniform grid of points representing the contact points of the electrical leads from which biopotential measurements were obtained. The biopotential leads are measured between the contact points of the sensing probe contacting the cardiac tissue. If the lead probe happens to be floating in the blood stream and not touching the cardiac tissue then the tissue impedance parameters defined by the model coefficients (poles and zeros of the transfer function) will indicate a markedly different pole-zero map as compared to the impedance parameters defined by the model coefficients representing biopotential measured from the cardiac tissue directly.

An electric biopotential Source and biopotential Sink location map is indicated on a display to the user as a color heat map which is co-located with anatomical features and spatial location in three dimensions. The color-heat map is an indication of how strong of an biopotential signal source or sink the tissue is at a particular location, as determined by the integral of the source/sink signal.

Electric biopotential and current can also be sensed in the blood stream, however, the impedance of the blood stream is markedly different than the impedance of the muscle tissue, as represented in the modeled transfer function coefficients or poles and zeros which have a different location pattern reflecting different tissue impedance properties and conduction characteristics between the input signals and output signals. Therefore, we can advantageously use this method to differentiate between a blood and tissue contact points between the contacts of an input lead and the contact of an output lead. This invention uses the poles and zeros of the transfer function, identified using the system identification methods, preferably state-space to indicate the impedance of the medium or tissue conducting the input signal into the output signal, and guides the user to whether it is representative of cardiac muscle tissue or blood stream. In another embodiment, the electric impedance is measured directly and a decision support system that classifies the impedance measurement as being representative of muscle medium or blood medium guides the user or clinician in placement of the electrodes contact points for purposes of measurement or therapy.

The procedure described above is repeated below in simplified steps:

1—Select target lead to be tested if contributing to an unknown biopotential source or biopotential sink. A target lead is typically oriented along a line, and generally approximately parallel to other reference leads, Ref1, Ref2, . . . , Refn (or simply Refx), also typically oriented along a line, where x=1, 2, . . . n. Perform this step and all the subsequent steps for all x reference leads which are selected to localize the region of interaction of the target lead and reference lead(s).
2—Identify using system identification methods, a linear or nonlinear transfer function relationship, preferably state-space linear model, between EGM target biopotential and EGM reference biopotential Refx. In a subsequent step, estimate said Refx output signal using the identified transfer function model and the EGM target input signal. Then compute (EGM Residual x)=(actual measured EGM Refx)−(estimated EGM Refx). If path between EGM biopotential target lead and EGM biopotential Ref1 lead is pure conduction path, then estimate should have a minimum spectral content in the EGM Residual1 signal, otherwise there are external sources interacting and adding to or subtracting from (neutralizing) the spectral content of the output Refx signal. In another embodiment, the transfer function configuration can be preferably selected as MIMO, MISO, SIMO type models which can more advantageously define a region of reference lead(s) interactions between the target lead(s) instead of a single reference lead and single target lead as in the previously described SISO model.
3—If the integral (area under the curve) of Residual x has a positive value above a minimum threshold, then the estimated target lead is experiencing interaction with external biopotential sources, and if the Residual x has a negative value below a maximum threshold, then the estimated target lead is experiencing external biopotential sink.
4—The region of interest can be expanded (zoomed-out) or detailed (zoomed-in) to detail the full region of normal tissue versus abnormal tissue and their interaction, and the results are displayed to the user for clinical assessment and therapy determination.
5—Optionally, a relationship transfer function can be established with the between the internal cardiac electrogram biopotential (EGM) leads signals, and the body surface Electrocardiogram (ECG) biopotenial leads. The relationship transfer function can use EGM as input or output and ECG as output or input, respectively. This step enables the mapping via this transfer function of either normal an abnormal leads to surface ECG so we can better understand the presentation of the abnormality on the surface ECG, given specific abnormal pattern in comparison to normal pattern. These mapping transfer functions are defined using linear or nonlinear system identification methods, preferably using linear state-space models.

Mapping Biopotential Tissue Sources, Sinks, and Circuits:

FIGS. 31 (a), 31 (b), 31 (c), 32 (a), 32 (b), 32 (c), 33(a), 33(b), 33(c), and 33(d) relate to the following discussion.

With each successive step in identifying the characteristics of a region of tissue in electrical signal conduction and stimulation or prevention which determines the role this tissue plays as either normal conductive tissue, or as an abnormal blocking tissue, a stimulating tissue, or as a delaying tissue. This process enables the generation of a map of the probe location relative to the patient body, or body's organ, or heart, or a vascular system artery or vein, the heart, the lungs, the aortic artery, the pulmonary vein and pulmonary artery, and ascending vena cava, and descending vena cava, and central venous, and jugular, and the carotid, and the external or internal cardiac muscle features including valves and chambers, and its structure and geometry. The probe navigation in the body can function similarly to the GPS system by using timed radio frequency waves or acoustic frequency waveforms, preferably outside of the audible spectrum, that is transmitted to or from the probe from or to fixed receivers or transmitters that are located in space outside of the patient body with a minimum of three such fixed points in space for receivers and/or transmitters (or simply transceivers). A series of radio or acoustic signal pulses, preferably square waves, are transmitted and received between the probe and the fixed transceivers. The relative time of flight of the transmitted or received signal between the probe and the fixed transceivers will indicate the position of the probe at all time. A fourth fixed transceiver will provide depth information of the probe. In one embodiment, radio waves are used whereby each lead acts as an antenna that either transmits and receives radio waves or modulated radio waves, preferably amplitude or frequency or phase modulated, and more preferably producing a discernible level shift such as a square wave with a selectable duty cycle and period, of spectral frequency content that is appropriate to the wavelength the small distances representative of external transceiver distance from the patient, such as 1 meter to 10 meters. In another embodiment when acoustic waves are used, the transceiver has a piezoelectric crystal that is above of converting electric waveform to/from sound waveforms. The probe is advanced into the patient body using a catheter, preferably using a guide wire, to the location of interest on the myocardium.

The catheter probe also comprises a plurality of electrodes wires forming contacts that measures the biopotential electrogram (EGM) on desired locations of the myocardium or vascular blood vessels or within cardiac chambers. Preferably, the probe, as depicted in FIG. 25 label 36, comprises of flexible electrodes wires, preferably insulated from each other with electrically insulating sheets, and when extended out of the probe lumen will form the measurement leads and contacts for biopotential measurement. Preferably, the plurality of electrode wires will form an umbrella, as depicted in FIG. 25 label 38, shaped wire guided grid that extend out of the shaft forming the catheter probe's lumen when an electrical measurement of the EGM biopotential is desired. The wire grid opens up as it is extended to create a concave pattern (umbrella) of a plurality of wires grid, each wire grid having a plurality, one or more, of electrical conductive contact points that are capable of communicating their sensed signals through the probe's lumen to the probe's connected biopotential measurement electronics. In one embodiment, the lead wires umbrella have a plurality of interconnections, preferably flexible, for structural integrity and support of the concave shaped wire grid. In one embodiment each electrode wire is formed by a flexible thin wire that is formed into a sheet that is made of either fiber optic or plastic or nylon type sheet. The plurality of the wire grid can be extended as a group and retracted as a group to form the umbrella shape. Each wire can flexibly bend to adapt to different cardiac tissue curvature and varying lengths across the tissue walls where biopotential is measured at. In a preferred embodiment the inner part of the plurality of sheets are made from fiber optics that conduct a source light into the cardiac chambers and blood vessels, and enables a formation of a visual picture with a camera of the internal structures of the cardiac muscle, and also a can provide for pulse oximetry measurement of oxygen content and saturation and hemoglobin value by conducting red and infra-red light wavelengths. In a preferred embodiment, the transmitted light through the fiber optics can provide images of the internal heart chambers in specific light wavelengths such as within the visible spectrum, in infra-red frequency range, or ultra-violet frequency range, or preferably at a frequency that is not absorbed by the blood but is absorbed by the muscle tissue, to render blood fluid invisible, blood or light with specific polarization of the wavelength, such as vertical or horizontal polarization, that is useful in detection of status of tissue scarring and its locations or to make visible any cardiac muscle or blood vessel tissue abnormalities. In a preferred embodiment said wavelength is X-ray radio waves and said wire lead is a conductive wire guide for said radio wave. In another preferred embodiment, said fiber optic sheets conduct laser light that can indicate the location of the probe's electrode sheets and their contacts, which can also imaged with the micro-electronic camera attached to the probe itself or to the probe's electrodes fiber optic sheets. In another embodiment said laser conducted within the fiber optics can be used for ablation of the abnormal tissue to cease its role in impacting normal rhythm or abnormal rhythm generation.

Each of the electrode wires also has the ability to locate itself relative to the probe or relative to the external fixed transceivers using either radio waves or acoustic waves that defines the location of each lead wire contact points or tip individually within the patient body or body's organ or heart. When acoustic waves are used, the speed of sound in the medium along with triangulation of one transducer's position or distance relative to other leads or the probe is considered to estimate the electrodes themselves will be able to function as individual probes transmitting and receiving the electric radio or acoustic wave with the fixed positioning transceivers. In one embodiment the radio frequency transmitted in the same wire performing the biopotential measurement is outside the bio-signal spectral content so the same wire can be used for measurement and location at the same time, yielding advantageously more precise anatomical spatial position and electrical measurement co-localization.

Computerized software systems will display a map of the cardiac muscle tissue depicting the electric function relative to the position of the probe to indicate an anatomic image of the area explored or mapped by the probes or plurality of probes. Merging of the electric anatomic map, preferably with color heat map indicating electrical sources and sinks, with a physical anatomic images of ultrasound, MRI, or CT, or X-Ray, or nuclear medicine images, or by visible light camera images is also preferable to enable a physiologic understanding of the underlying physiology with the electric mapping functions. Variable degree of spatial resolution in the electrical mapping process can be selected from coarse to fine to enable a zoom-in or zoom-out about the region of interest in the electrical map to understand its characteristics and fine details. This process of zoom-in/zoom-out advantageously allows a rapid convergence to a particular abnormal tissue region of interest to define its physical boundaries and electrical characteristics and relationships to other normal or abnormal tissue by comparison as well as its relationship to physical anatomic features defined by anatomic imaging methods including MRI, CT, Ultra-sound, X-ray and functional imaging methods including nuclear medicine, SPECT, or PET imaging methods. This zoom-in/zoom-out approach enables a hierarchical mapping process to rapidly eliminate measurement locations or regions outside of the interest area, which may not be useful for the physician in the treatment process itself and may waste valuable time during the surgical procedure.

Cardiac Arrhythmia or Fibrillation Neutralization, and Evoked Potential Pacing Therapy:

Another object of this invention is to define novel therapeutic methods for arrhythmia and fibrillation treatment by applying electric biopotential neutralization (EBN) either globally or locally to the abnormal tissue. Please refer to FIGS. 31(a), 31(b), and 31(c) for the following discussion. Global correction to the abnormal rhythm involves the following steps:

To apply therapeutic treatment of identified abnormal rhythm, and using a plurality of measurement electrodes, the following steps are applied.

1—Step 3101, Measure EGM on cardiac muscle or major vessels or blood using plurality of probe's lead wires.
2—Step 3102, Map or transform EGM leads to standard body surface ECG leads via transfer function that is identified using said linear or nonlinear system identification methods, preferably linear state space.
3—Step 3103, Identify ECG rhythm correction desired with a target waveform morphology, a template, that would be targeted or desired for the body surface ECG.
4—Step 3104, Use model free adaptive control to derive the corrective ECG waveform that would neutralize the abnormal ECG waveform components and render the target template normal ECG waveform components, or simply subtract the target desired waveform from the actual ECG to identify the difference waveform between them which will be described here in as the Correction Waveform (CW).
5—Step 3105, Generate the Correction Waveform stimulus pulse and apply on same electrodes measuring the ECG locations turning the ECG sensing electrodes into stimulation contacts for CW application that produces ECG rhythm correction waveform (ECG CW) on measured ECG.
6—Step 3170, The ECG CW stimulated correction waveform will transfer using tissue characteristics directly to the target EGM to apply a corrective signal on the target EGM (EGM_CW). The transfer function that models the relationship between the body surface ECG as an input waveform and intra-cardiac EGM as an output waveform can be identified adaptively using said linear or nonlinear system identification methods, preferably linear state-space methods. The EGM_CW will define the local electric waveform correction desired in order to affect the surface body measured ECG according to the target desire template waveform morphology, thus neutralizing abnormal waveform components. Induced correction or localized EGM stimulus (EGM_CW) at myocardial lead can be measured and for localized corrected EGM can be evaluated for efficacy. Residual error can be provided into a closed loop feedback into an adaptive control system, preferably model free adaptive control, that fixes the correction EGM_CW applied until the residual error is minimized, or desired target body surface ECG is obtained. The tissue impedance typically will reduce the function to reduce current and maximize voltage, so in order to make induced EGM correction waveforms effective, the applied current level on the body surface ECG electrodes will need to be high enough to overcome tissue impedance to produce a current level that is effective locally in neutralizing abnormal EGM components.
7—Alternatively apply EGM rhythm correction directly on (body) surface ECG lead (higher current levels may be required to transfer applied lead potential at skin surface to effective neutralizing EGM signal on the cardiac muscle).
8—In a preferred embodiment, a plurality of leads are implanted into the cardiac muscle for pacing the muscle with a localized rhythm corrective EGM_CW that is continuously adapted to produce a target desired template pattern of body surface ECG waveform morphology. The local EGM is measured (at site 3152, step 3162) and simultaneously the body surface ECG is measured (at site 3151, step 3165), a linear or nonlinear system identification method, preferably linear state space, is used to define the relationship transfer function between surface ECG (as input) and EGM (as output) (3163, 3101, 3102), the measured ECG is compared to the target ECG waveform morphology (3103), and a closed loop feedback controller 3166, preferably using a model free adaptive controller (including MFA (defined in U.S. Pat. No. 6,055,524 included herein by reference) or a PID controller (proportional integral differential) to provide feedback correction waveform 3167 of measured waveform 3164 (process variable) to achieve target waveform 3171 (set point), or alternatively simply a subtraction of the template from the actual measured ECG, a corrective ECG CW is produced (3161, 3104), then the ECG CW is applied to the previously adaptively identified transfer function to produce a target EGM_CW, then the EGM_CW is used to correct the arrhythmia or abnormality in cardiac rhythm or fibrillation by locally applying EGM biopotential (3168, 3105) with a stimulating current level to induce a local EGM correction 3169 that results in the desired target rhythm 3170 as measured at the body surface ECG.

In another embodiment, the local EGM (biopotential signal on cardiac muscle) correction to the abnormal rhythm involves the following steps:

1—Step 3165, Obtain EGM measurement on a myocardial lead.
2—Identify local EGM rhythm correction 3167 required directly and define desired target waveform morphology.
3—Step 3166, Apply closed loop control systems, preferably model free controller (MFA or PID), to provide feedback correction waveform 3167 of measured waveform 3164 (process variable) to achieve target waveform 3171 (set point), or simple subtraction of desired target waveform 3171 from actual measured EGM waveform 3164 to define the EGM_CW 3167 required to apply by stimulating the tissue to produce the desired target (correct) rhythm locally as measured by the EGM. The EGM measuring leads in this case can be at proximity, preferably within 1 mm, to the EGM stimulation leads on the cardiac muscle. Application of a corrective EGM by local stimulus at the myocardial lead will produce a localized component correction which requires only small current levels to induce such correction effectively.

EEG, EMG, and Neural Modulation or Stimulation Therapy:

Similar equivalent arguments to the entire discussion and spirit of the present invention of leadless ECG, can be applied to these other bioelectric physiologic signals of neurological interest. The spirit of this instant invention, without loss of generality, can be equivalently applied and extended to other frequency ranges or spectral bands, and topographic area of measurement to be applied to EEG, EMG, or EOG. Similar electronic components and wireless transmission protocols and informational exchange can be applied for measurement of such bioelectric physiologic signals.

More specifically, for leadless EEG (electroencephalogram) application the system, algorithms and methods described above for leadless ECG can be extended for equivalent application to EEG electrophysiologic waveforms. While EEG has a different frequency spectral band than ECG, the system identification methods, preferably linear state space method, model tissue bioimpedances as the system linking two leads, and not the frequencies content within the input and output waveforms of the model. Therefore while keeping the structure of the model fixed (i.e. linear state space model) the model order and coefficients will vary to describe the behavior and characteristics of different tissue types or different tissue segments of the same type which are represented by different tissue bioimpedances being modeled and therefore different states and differential equations. The biopotential measuring system design or stimulation system design need to take into account modifications required for amplification, filtering, and digitization operating at a different frequency spectral band and different waveform amplitudes, including filtering frequency band, number of channels, and amplification gain to make it fit for the EEG application.

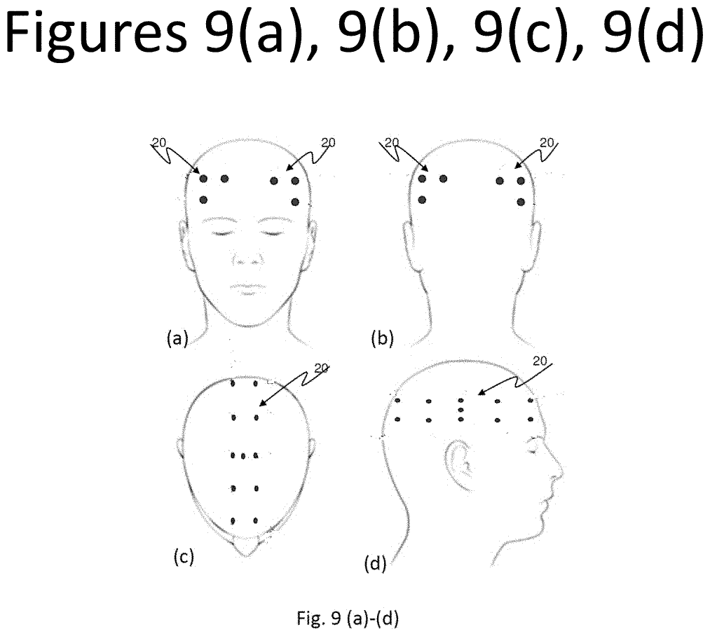

In FIGS. 9(a) through 9(d), there are illustrated various locations that the electrode assembly of the present invention may be placed on the head's scalp surface of the patient. One of the electrode assembly's plurality of contact points serves as a reference electrode contact point for the remaining contact points. The leadless EEG device can equivalently be applied for the measurement of EEG as a bio-potential for the purpose of creating inter-channel mapping transfer functions (models defined using system identification methods, preferably linear state-space models). These mapping transfer functions can be used to estimate one EEG lead as a transfer function output using one or more EEG leads as transfer function inputs, and therefore significantly reduce the number of acquired EEG leads during real-time EEG monitoring by supplementing with estimated or calculated EEG leads (as model outputs) using a reduced or minimal number of acquired short EEG leads (as model inputs). Furthermore, estimated EEG leads can be used to further estimate other EEG leads as needed, with accumulative residual estimation error nonetheless.

By reducing the number of acquired EEG channels to form a full montage down to one, two, or three channels, for example, one can substitute the measurement of the remaining EEG channels by a calculation of the estimated EEG for these channels using the minimal set of measured EEG channels as input and a plurality of identified system models relating the measured input channels with the remaining output channels.

Calculated standard lead EEG waveforms can be estimated using the input short lead EEG waveforms and a system model of transfer function relationship between the standard lead (as model output) and an input short lead, such as that acquired with a wearable sensor for example. Alternatively, The short lead used as input could also represent another standard lead itself as needed. In other words, some of the estimated output standard leads can be used in a subsequent step as input EEG leads into a secondary system identification process step, in order to reduce estimation errors and better identify a secondary transfer function model, preferably linear state-space model, to estimate other standard EEG leads as its outputs. Therefore, with the models estimated available, a few EEG standard leads can be then measured, and the rest can be estimated with fairly high accuracy. This will allow for significantly reduced number of leads for acquisition of full EEG montage leads.

It is understood to those skilled in the art that the above discussion on ECG and EEG can be equivalently applied for EGM, EMG, EOG, or other Neural biopotentials, without loss of generality.

In addition, the conventional direct (linear or non-linear) or blind system identification method previously described can be applied in a novel application for monitoring of bio-potential activity generation sources in the brain. A plurality of measured bio-potentials on each hemisphere of the brain can be used adequately to estimate the generating source signals and location of the signals. This can be accomplished by using “short-lead” signals to estimate other leads as output of system identified models, then comparing the model output signals with actual measured signals from such modeled leads. The comparison yields difference signals representing unique or novel informational components for these channels while removing common informational components. This method can be used to isolate these components as being sourced from either the frontal lobe, or the posterior lobe, or from either the left hemisphere, the right hemisphere, or classified as being crossing over from one hemisphere to the other. Such segregation is important for isolation of the source of abnormal bio-potential activity, and then the determination of its location. Sources of abnormal bio-potential activity can include tumors, seizure activation sites, Parkinson source trigger tissue, Alzheimer reduction and asynchronization in electric activity, and/or effect of sedative medications on each of the hemispheres as well as aggregate effect estimation.

In a similar application to aforementioned application of ECG arrhythmia and fibrillation therapeutic treatment and correction, brain abnormalities represented by EEG waveform abnormalities can also be corrected and treated. Brain abnormalities include, but are not limited to, seizures abnormal stimulation, and Parkinson related tremors, can be neutralized or substantially reduced by application of stimulation pattern that represents the reverse or neutralization of these undesired or abnormal stimulation frequencies.

An object of this invention is to define novel therapeutic methods for seizures and Parkinson treatment by applying electric biopotential neutralization (EBN) either globally or locally to the abnormal tissue. Global correction to the abnormal rhythm involves the following steps to apply therapeutic treatment of identified abnormal rhythm, and using a plurality of measurement electrodes, the following steps are applied:

1—Measure using a plurality of probe's lead wires localized neural biopotential on or within the brain tissue, or in major brain blood vasculature vessels or brain chambers, this defined biopotential is herein called a Neurogram (NGM).
2—Steps 3101 and 3102, Map or transform NGM leads to standard biopotential encephalograph (EEG) leads measured or acquired on the patient's head surface simultaneously to the NGM. Adaptively identify a relationship transfer function between the NGM (as transfer function input or output) and EEG (as transfer function output or input, respectively) using said linear or nonlinear system identification methods, preferably linear state space models.
3—Identify EEG rhythm correction desired 3161, typically preferably we would target neutralization of EEG to zero due to its general unpredictability and lack of periodicity, leading to a target waveform that would force the abnormal biopotential (signal) waveform pattern to be neutralized or substantially reduced for the duration of the abnormal event or for a user defined duration or pre-determined duration. The correction biopotential waveform 3171 that would neutralize the abnormal EEG pattern to achieve the target, which is preferably a zero biopotential voltage across the measured EEG electrode contacts, to be applied to the patient's head surface EEG electrode contacts. The stimulation leads contacts are at proximity, preferably within 1 mm, to the EEG measurement leads.
4—(Step 3104) Use model free adaptive control 3166 to derive the corrective EEG waveform 3167 that would be injected 3105 and propagate across tissue impedances 3171 to neutralize the abnormal EEG waveform components and render the target template normal EEG waveform components. Alternatively, simply subtract the target desired waveform 3164 from the actual measured EEG 3161 to identify the difference waveform between them which will be described here in as the Correction Waveform (CW) 3167.
5—Generate the Correction Waveform stimulus biopotential waveforms using a signal generator and apply the CW biopotential on stimulation electrodes contacts for CW application 3168, which produces EEG rhythm correction waveform (EEG_CW) on measured EEG.
6—The EEG_CW stimulated correction waveform will be conducted using tissue impedance characteristics 3169 directly to the target lead on the brain tissue with abnormal measured NGM where we apply the equivalent corrective signal to the target tissue NGM (NGM_CW) 3171. (3101, 3102) The transfer function that models the relationship between the head's surface EEG as an input waveform and intra-brain NGM as an output waveform can be identified adaptively using said linear or nonlinear system identification methods, preferably linear state-space methods. The NGM_CW will define the local electric waveform correction desired in order to affect the head's surface measured EEG according to the target desire template waveform morphology, thus neutralizing abnormal waveform components. Induced correction or localized NGM stimulus (NGM_CW) at a brain tissue lead can be measured 3161 and for localized corrected NGM can be evaluated for efficacy. Residual error can be provided into a closed loop feedback into an adaptive control system 3166, preferably model free adaptive control, that fixes the correction NGM_CW applied 3167 until the residual error is minimized, or desired target head's surface EEG is obtained. The tissue impedance typically will function to reduce current and maximize voltage, so in order to make induced NGM correction waveforms effective, the applied current level on the head's surface EEG electrodes will need to be high enough to overcome reductions due to tissue impedance to produce a local current level that is effective enough in neutralizing abnormal NGM components.
7—Alternatively we can apply NGM rhythm correction directly using surface EEG lead, however, higher current levels may be required due to current losses with tissue impedances between the application contacts on the brain surface to produce the desired correction pattern at the local NGM lead site.
8—In another preferred embodiment, a plurality of leads are implanted into the brain tissue for stimulating the tissue with a localized rhythm corrective NGM_CW 3167 that is continuously adapted to produce a target desired neutralization of the body surface EEG waveform morphology 3171. In Step, 3165 The local NGM is measured and simultaneously the head's surface EEG is measured, a linear or nonlinear system identification method, preferably linear state space, is used to define the relationship transfer function between EEG (as input) and NGM (as output) 3163, the measured EEG 3164 is compared to the target EEG waveform morphology 3161, and using close loop controller 3166, preferably a model free adaptive controller (such as MFA (defined in U.S. Pat. No. 6,055,524 included herein by reference) or PID (proportional integral differential), to provide feedback correction waveform 3167 of measured waveform (process variable) to achieve target waveform (set point) 3161, or alternatively simply a subtraction of the target EEG waveform 3161 (including zero electric biopotential) from the actual measured EEG 3164, a corrective EEG_CW 3167 is produced (i.e. negation of abnormal EEG pattern), then the EEG_CW is applied 3168 to the previously adaptively identified transfer function to produce a target NGM_CW 3167, then the NGM_CW is used to correct the abnormality biopotential rhythm in brain by locally applying NGM biopotential 3169 with a stimulating current level 3167 to induce a local NGM correction that results in the desired target desired biopotential waveform 3171 which can also be measured at the body surface EEG.

In another embodiment, the local NGM correction to the abnormal rhythm involves the following steps (applicable subset steps from FIGS. 31(a), 31(b), 31(c) are indicated):

1—NGM measurement on a brain tissue lead.
2—Step 3162, Identify local NGM rhythm correction 3161 required directly and define desired target waveform morphology. In a preferably embodiment, the desired target 3161 is neutralizing the local NGM to zero biopotential for the duration of the treatment application.
3—Step 3166, Apply closed loop feedback controller, preferably model free controller (MFA or PID), to provide feedback correction waveform 3167 of measured waveform (process variable) 3164 to achieve target waveform (set point) 3171. Alternatively, a simple subtraction of desired target 3171 from actual measured NGM 3164 to define the NGM_CW 3167 required to apply by stimulating the brain tissue to produce the desired target (correct) rhythm locally as measured by the NGM. The NGM measuring leads in this case can be at proximity, preferably within 1 mm, to the NGM stimulation leads on the brain tissue. Application of a corrective NGM by local stimulus at the brain tissue lead will produce a localized component correction which requires only small current levels to induce such correction effectively.

Severed Neural Pathway Conduction:

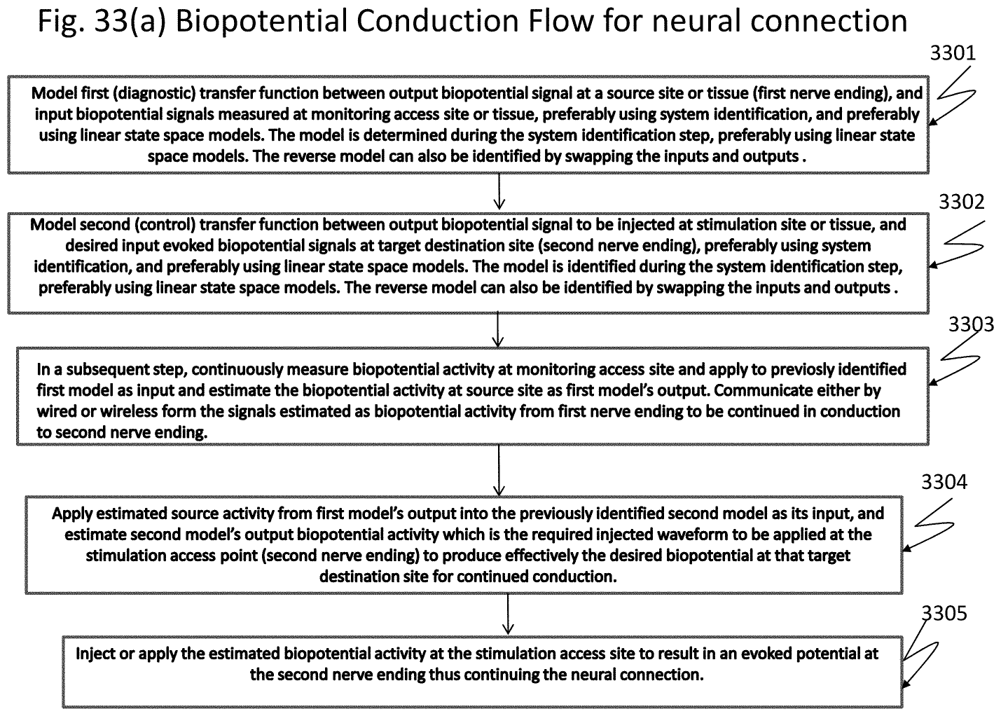

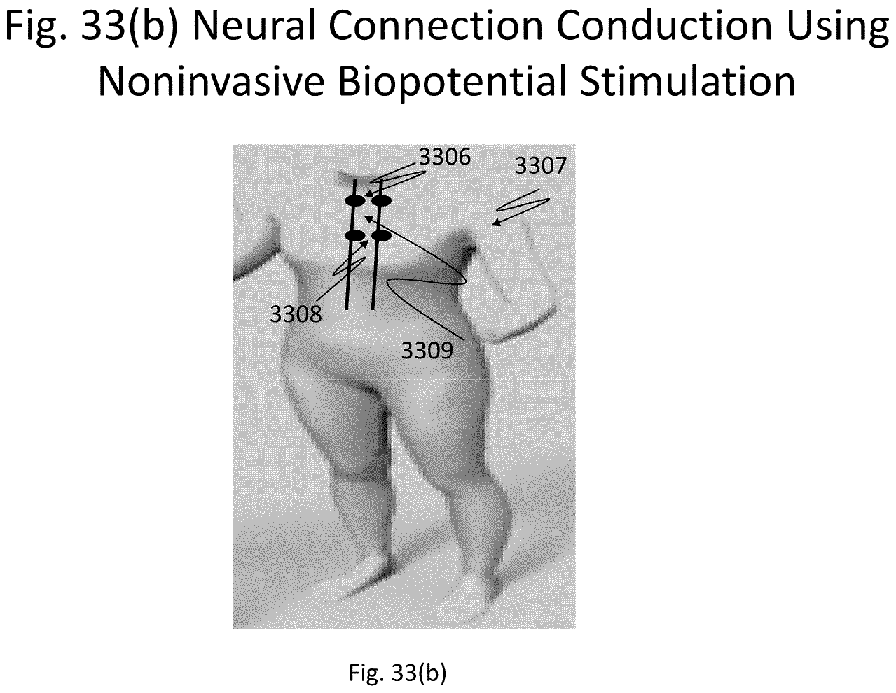

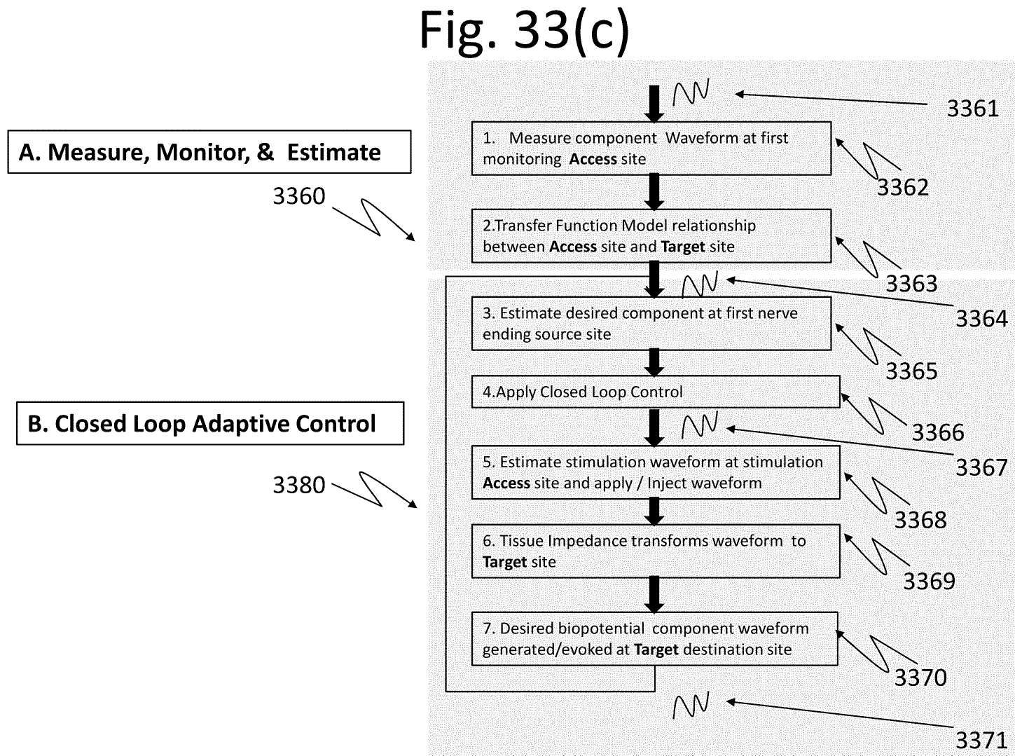

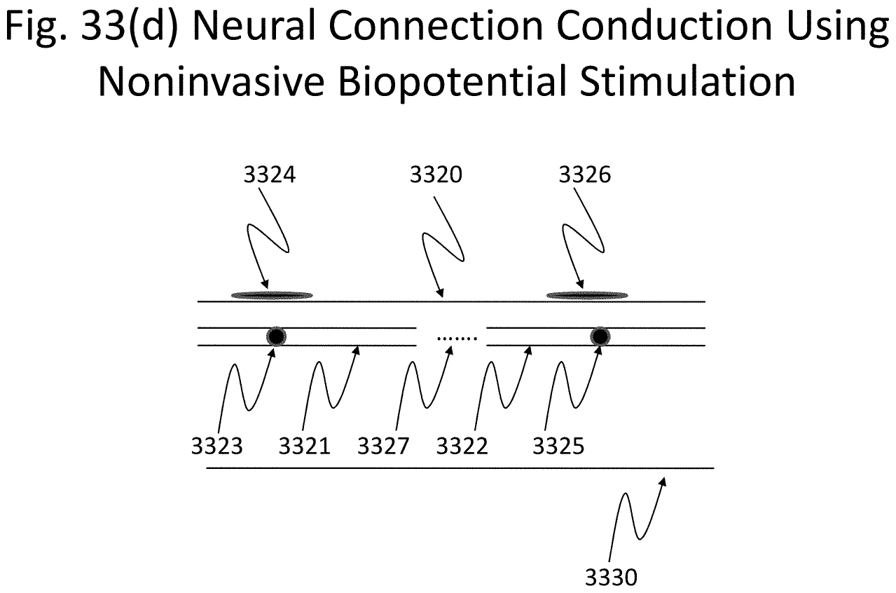

The following discussion relate to FIGS. 33(a), 33(b), 33(c), and 33(d).

Electrical Anesthesia:

Another object of this invention is to define the application of above mentioned stimulation and control for effective electrical current neutralization in the brain tissue using electrical leads to produce electrical anesthesia (or E-ANESTHESIA) which results in effective global neutralization of the brain activity using external stimulation of biopotential at a plurality of leads contact points that are applied to the patient's head surface. This is advantageous to the current practice of chemically induced anesthesia in that it has substantially lower risk on the brain tissue health, and is applied on demand and for the desired duration of the surgical procedure, effectively acting to switch patient's state of consciousness on-off on demand by substantially neutralizing (i.e. substantially zeroing) the EEG brain activity (similar to Propofol anesthetic effect on brain function), or alternatively by randomly exciting with gaussian white noise content in the EEG signals the brain (similar to Nitrous Oxide anesthetic effect on brain function). This process effectively leads to substantially instantaneous recovery for the patient and does not lead to long term side effects of the drugs such as memory loss, or potentially death due to the adverse drugs side effects. Also the EEG electrical biopotential neutralization waveform can be reduced to a (minimal) level (above zero) that does not result in respiratory suppression by not completely neutralizing that region in the brain. This can be accomplished by gradual increase in the amplitude of the complete neutralization biopotential until loss of consciousness is achieved without respiratory suppression. The recovery process can be applied (by reducing EEG suppression corrective stimulation) instantly or gradually based on the preference of the clinician for pain management and muscle relaxation and patient's brain recovery performance. The above can also be used for pain treatment applications for medical reasons and muscular relaxation of patient for medical and non-medical applications.

As thus far described, the leadless ECG (or leadless EEG) system of the present invention models the patient's body's ability to conduct ECG (or EEG, respectively) and the effects of varying skin impedances. The identified parameters and models describe the transfer functions (system models) between the patient's measured bio-potentials at specific points on the surface of the skin (or internal to it if electrodes are subcutaneous or implantable). However, dynamic changes to these modeled parameters can also be indicative of the dynamic state changes in the patient's body over time, given fixed measurement positions of the electrodes contacts. Automated remodeling of the patient's transfer function (system model) relating the skin surface ECG's (EEG's) allows for dynamic noninvasive monitoring of the patient's body dynamics. Dynamic changes in the identified modeled parameters that describe the patient's body can be used as non-invasive indicators used to frequently monitor physiologic factors that influence such model parameters. Some of these factors are cardiac output, and hydration status level, and effects of induced vasoconstriction or vasodilatation of the vascular system.

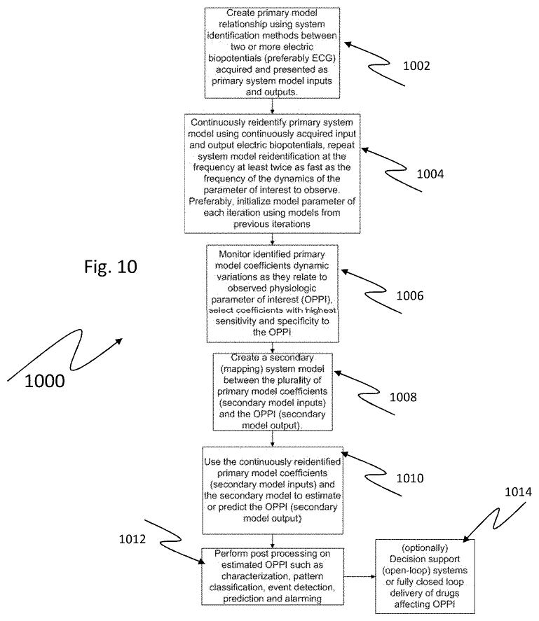

With attention directed to FIG. 10 of the drawings, there is illustrated a flow diagram 1000 of an exemplary embodiment of a physiologic parameter monitoring procedure for dynamic non-invasive monitoring of the patient's body dynamics. In step 1002, identification of a primary model (structure, and order) and its parameters (values) that best-fits a physiologic relationship between selected plurality of input physiologic variables or input analytic variables and selected plurality of output physiologic variables or output analytic variables is created. Such relationship between inputs and outputs has to be meaningful physically, chemically, or electrically for the model to reflect reality. Model optimization by selecting the right model order and model structure that minimizes output estimation errors from actual outputs is important.

In step 1004, repeatedly re-identifying the model parameters (using same model structure and order as primary) utilizing new information of the plurality of inputs and plurality of outputs at same measurement locations as before is continuously and repeatedly re-identified. Model re-identification occurs at a frequency and interval that are meaningful for the underlying model change. The model sampling frequency is required to be at least twice as fast as the expected change in dynamics of the model parameters in order to obtain representative reflection of the dynamic system changes that is presented in the model parameters variations. The previously identified model may always be used as part of the initial conditions for re-identifying the model parameters or optimizing its parameters goodness of fit as measured with estimated outputs difference from actual outputs.

Each of the parameters within the identified model structure for dynamic variations through time that are representative or correlating with the dynamic variations in target physiologic or analytic parameters of interest is monitored in step 1006. In other words, do any of the system model parameters reflect underlying system model changes (as opposed to input changes) that offer a correlating relationship with a target physiologic or analytic parameter. For example, the system relating a plurality of ECG waveforms describes or maps the underlying tissue electric properties and those properties changing through time may be correlated with a number of variables of interest. For example, a plurality of model parameters may offer more sensitivity and specificity to cardiac output or fluidic hydration status that is altering the electrical properties of the tissue within which the ECG is measured by variable hydration state. Another parameter of the identified model may offer more sensitivity or specificity to pain or stress experienced by oncology patients or patients undergoing surgery. Furthermore, another parameter of the identified model may offer greater sensitivity and specificity to glucose level in a patient.

In step 1008, a secondary identified model function is created so to relate at least one of a plurality of the primary model parameters variation over time and a plurality of the primary model state functions to the desired target physiologic or analytic parameter (or “observed physiologic parameter of interest”—OPPI) with high sensitivity and specificity. This allows one to effectively estimate and predict the desired target physiologic or analytic parameter using only the inputs and outputs required for the primary model identification and continuous re-identification at a selected frequency. For example, use ECG bio-potential waveforms input and output to monitor glucose, or respiration, or cardiac output, or hydration status.

In step 1010, For example, using ECG bio-potentials as input(s) and output(s) into the primary system identified model parameters, one can determine parameters with variations or dynamics that are most sensitive and/or specific to chest fluidic content, and can therefore use that for detecting abnormal events occurrence such as detection of congestive heart failure conditions. Similarly, one or more coefficients of the best fit system identified model, preferably state space, can be linked with its sensitivity and specificity to glucose as its concentration changes over time, and can therefore identify a relationship between the varying glucose concentration and variations to one or more coefficient(s) describing the system model underneath.

Such relationship between a desired observable (desired output) physiologic or chemical effect on the system represented by a variation of the (measured input) system model coefficients over time can further be well defined by a secondary system identification step relating the two input and output variables. Therefore, the primary system identification step 1002 provided a description of the physiologic and anatomic system between non-invasively measured bioelectric surface potentials, and the repeating of such system description provides insights into its variation over time due to some parameter that is desired to be observed. On the other hand, the secondary system identification step 1008 establishes such relationship between the varying system-identified coefficients of the model and the desired varying observable so that we can predict the later (as output) using the former (as input) into the secondary system-identified model.

Post processing on the estimated observed physiologic parameter of interest, such as characterization, pattern, classification, event detection, prediction, and alarming is performed in step 1012. Finally, in optional step 1014 decision support (open-loop) systems or fully closed loop may be used to provide delivery of drugs affecting the observed physiologic parameter of interest.

While there has been illustrated and described what is at present considered to be a preferred embodiment of the present invention, it will be understood by those skilled in the art that various changes and modifications may be made, and equivalents may be substituted for elements thereof without departing from the true scope of the invention. In addition, many modifications may be made to adapt a particular situation or material to the teachings of the invention without departing from the central scope thereof. Therefore, it is intended that this invention not be limited to the particular embodiment disclosed as the best mode contemplated for carrying out the invention, but that the invention will include all embodiments falling within the scope of the claims.

### APPENDIX

### References

1. System Identification: Theory for the User, Lennart Ljung, Prentice Hall, 2nd edition, 1999.
2. System Identification: A Frequency Domain Approach Rik Pintelon and Johan Schoukens Wiley-IEEE Press, 1st edition, January, 2001
3. Blind Equalization and System Identification: Batch Processing Algorithms, Performance and Applications, Springer, 1st Edition, January 2006.
4. Linear estimation, Kailath, Sayed, Hassibi. Prentice Hall, 2000.
5. Multivariable System Identification For Process Control, Y. Zhu, Elsevier Science; 1 edition (October, 2001)
6. Modeling of Dynamic Systems, L. Ljung, Prentice Hall; 1 edition, May 1994.

What is claimed is:

1. A biopotential measurement and calibration system for leadless electrocardiographic (ECG) measurement of electrical activity of the heart in a subject's body, the system comprising:
at least one multi-contact bio-potential electrode assembly adapted for attachment to the subject's body, said electrode assembly being formed of an electronic layer and an electrode layer;
said electrode layer having a plurality of contact points for engagement with the surface of the subject's body and configured to measure short-lead ECG signal(s) in response to electrical activity of the heart;
a calibration probe having an interior conductive surface and an exterior conductive surface, wherein said calibration probe is capable of sensing one or more ECG biopotential calibration long leads when (1) said interior conductive surface is in contact with a patient's finger or wrist, and (2) said exterior conductive surface is in contact with patient's body; and
a processing unit configured to produce a transfer function during calibration based on the measured short-lead ECG signal(s) from said plurality of contact points and said one or more calibration long leads; and thereafter uses said transfer function to compute estimated calibration long-lead ECG signal(s) based on the measured short-lead ECG signal(s) from said plurality of contact points.

2. The system of claim 1, wherein said ECG biopotential calibration long lead represents a component of a closed Kirchoff's voltage loop with a standard ECG long lead.

3. The system of claim 1, wherein said ECG biopotential calibration long lead represents a standard ECG long lead signal.

4. The system of claim 1, further comprising a monitor in communication with at least one multi-contact electrode assembly, wherein said monitor is configured to receive at least one of said transfer function, measured short lead ECG signal(s), measured or estimated calibration long lead ECG signal(s), measured or estimated long lead ECG signal(s), measured or estimated standard long lead ECG signal(s), for displaying said ECG signal(s) and other meaningful information.

5. The system of claim 1, wherein an electrode assembly is coupled to a transceiver unit to receive at least one of said transfer function, measured short lead ECG signal(s), measured or estimated calibration long lead ECG signal(s), measured or estimated long lead ECG signal(s), measured or estimated standard long lead ECG signal(s), for processing said ECG signal(s) and other meaningful information.

6. The system of claim 1, wherein said leadless ECG system is wireless, said electronic layer includes a transceiver unit for transmitting and receiving wireless communications with a base station or a monitor, and said base station or monitor includes a wireless transceiver for transmitting and receiving communications with said contacts of the electrode assembly, wherein said wireless communications received by said wireless transceiver include at least one of said transfer function, measured short lead ECG signal(s), measured or estimated calibration long lead ECG signal(s), measured or estimated long lead ECG signal(s), or measured or estimated standard long lead ECG signal(s).

7. The system of claim 1, wherein said leadless ECG system is wireless, and said calibration probe includes a transceiver unit for transmitting and receiving wireless communications with a base station or a monitor, and said base station or monitor includes a wireless transceiver for transmitting and receiving communications with said calibration probe, wherein said wireless communications received by said wireless transceiver include at least one of said transfer function, measured short lead ECG signal(s), measured or estimated calibration long lead ECG signal(s), measured or estimated long lead ECG signal(s), or measured or estimated standard long lead ECG signal(s).

8. The system of claim 1, wherein said calibration probe is wireless, and said calibration probe includes a transceiver unit for transmitting and receiving wireless communications with a base station or a monitor, and said base station or monitor includes a wireless transceiver for transmitting and receiving communications with said calibration probe, wherein said wireless communications received by said wireless transceiver include at least one of said transfer function, measured short lead ECG signal(s), measured or estimated calibration long lead ECG signal(s), measured or estimated long lead ECG signal(s), or measured or estimated standard long lead ECG signal(s).

9. The system of claim 1, wherein said at least one multi-contact bio-potential electrode assembly is in communication with at least a second multi-contact bio-potential electrode assembly, said communications including at least one of said transfer function, measured short lead ECG signal(s), measured or estimated calibration long lead ECG signal(s), measured or estimated long lead ECG signal(s), measured or estimated standard long lead ECG signal(s), and other meaningful information.

10. The system of claim 1, wherein said processing unit is disposed in said electronic layer of said electrode assembly.

11. The system of claim 1, wherein said processing unit is disposed in said calibration probe.

12. The system of claim 1, wherein said processing unit is disposed in said base station or monitor.

13. The system of claim 1, wherein said transfer function is identified using a system identification method.

14. The system of claim 1, wherein said transfer function is identified using a system identification method employing a linear state-space model.

15. The system of claim 1, wherein said processing unit determines the need for a new calibration step to re-identify the transfer function.

16. The system of claim 1, wherein said transfer function computes estimated long-lead ECG signal(s) based on at least one other estimated long-lead ECG signal(s), measured long-lead ECG signal(s), or measured short-lead ECG signal(s) from said plurality of contact points.

17. The system of claim 1, wherein said processing unit employs signal processing and analysis on said measured and estimated ECG signal(s) to detect and indicate abnormalities in ECG rhythm or patient's health state.

18. The system of claim 1, wherein said electronic layer includes a plurality of electrical contacts for attaching a plurality of extended lead wires for measurement of a plurality of long-lead signal(s).

19. The system of claim 1, wherein said long-lead signal(s) represent standard ECG lead(s) with standard ECG electrode locations on the body.

20. The system of claim 1, wherein said electrode assembly is placed on top of or at proximity to the cardiac area, including next to or near the heart.

21. The system of claim 1, wherein said electrode assembly is placed within proximity to a fetal area of the maternal abdomen, and the bio-potential electrical activity contains fetal ECG (fECG) for monitoring fetal heart electrical activity.

22. The system of claim 1, wherein the bio-potential electrical activity represents evoked potentials (EVP) of any tissue.

23. The system of claim 1, wherein said electrode assembly is placed in contact with the heart muscle and the bio-potential electrical activity represents cardiac electrograms (EGM) for monitoring heart muscle activity.

24. A biopotential measurement and calibration method for leadless electrocardiographic (ECG) measurement of electrical activity of the heart in a subject's body, the method comprising:
providing at least one multi-contact bio-potential electrode assembly adapted for attachment to the subject's body, said electrode assembly being formed of an electronic layer and an electrode layer, said electrode layer having a plurality of contact points for engagement with the surface of the subject's body;
providing a calibration probe having an interior conductive surface and an exterior conductive surface, said interior conductive surface in contact with a patient's finger or wrist, said exterior conductive surface in contact with the patient's body;
measuring, using the at least one multi-contact bio-potential electrode assembly, first short-lead ECG signal(s) in response to electrical activity of the heart;
sensing, using the calibration probe, one or more ECG biopotential calibration long leads;
producing, using a processing unit, a transfer function during calibration using said measured first short-lead ECG signal(s) and said ECG bio-potential calibration long leads; and
computing, using said transfer function, estimated calibration long-lead ECG signal(s) based on second measured short-lead ECG signal(s) from said plurality of contact points.

25. The method of claim 24, wherein said ECG biopotential calibration long lead represents a component of a closed Kirchoff's voltage loop with a standard ECG long lead.

26. The method of claim 24, wherein said ECG biopotential calibration long lead represents a standard ECG long lead signal.

27. The method of claim 24, wherein said transfer function is identified using a system identification method.

28. The method of claim 24, wherein said transfer function is identified using a system identification method employing a linear state-space model.

29. The method of claim 24, wherein said transfer function is identified using a system identification method that initializes from a previous said transfer function.

30. The method of claim 24, wherein said processing unit determines the need for a new calibration step to re-identify the transfer function.

31. The method of claim 24, wherein said transfer function computes estimated long-lead ECG signal(s) based on at least one other estimated long-lead ECG signal(s), measured long-lead ECG signal(s), or measured short-lead ECG signal(s) from said plurality of contact points.

32. The method of claim 24, wherein said processing unit employs signal processing and analysis on said measured and estimated ECG signal(s) to detect and indicate abnormalities in ECG rhythm or patient's health state.

33. The method of claim 24, wherein said electronic layer includes a plurality of electrical contacts for attaching a plurality of extended lead wires for measurement of a plurality of long-lead signal(s).

34. The method of claim 24, wherein said long-lead signal(s) represent standard ECG lead(s) with standard electrode locations.

35. The method of claim 24, wherein said electrode assembly is placed on top of the cardiac area on the surface of the subject's skin.

36. The method of claim 24, wherein said electrode assembly is placed at proximity to the cardiac area, such as next to or near the side of the heart.

37. The method of claim 24, wherein said electrode assembly is placed within proximity to a fetal area of the maternal abdomen.

38. The method of claim 24, wherein said electrode assembly is placed on at least one of: the left shoulder area, left arm, left side, right shoulder area, right arm, right side, upper frontal area, upper frontal abdominal area, abdominal area, upper dorsal area, or dorsal area of the subject's body.

39. The method of claim 24, wherein said system is employed in conjunction with a standard ECG measurement system to improve performance against leads providing a noisy signal or disconnected leads.

40. The method of claim 24, wherein said estimated long leads are converted into analog output signal(s).

41. The method of claim 24, wherein the bio-potential electrical activity represents an electroencephalogram (EEG) for monitoring brain activity.

42. The method of claim 24, wherein the bio-potential electrical activity represents an electromyogram (EMG) for monitoring muscle activity.

43. The method of claim 24, wherein the bio-potential electrical activity represents fetal ECG (fECG) for monitoring heart activity.

44. The method of claim 24, wherein the bio-potential electrical activity represents evoked potentials (EVP) of any tissue.

45. The method of claim 24, wherein the bio-potential electrical activity represents a cardiac electrogram (EGM) for monitoring heart muscle activity.

46. The method of claim 24, wherein the bio-potential electrical activity represents an electroneurogram (ENG) for monitoring nerve activity.

47. The method of claim 24, wherein the variation from measurement time to calibration time in the biopotential estimation error for any lead is used to evaluate the health state of the patient, medication effect on patient, or a need for recalibration.

48. A biopotential measurement and separation method for measurement of electrical activities from two or more bio-potential sources in a subject's body, the method comprising:
providing at least one multi-contact bio-potential electrode assembly adapted for attachment to the subject's body, said electrode assembly being formed of an electronic layer and an electrode layer, said electrode layer having a plurality of contact points for engagement with the surface of the subject's body;
measuring, using the at least one multi-contact bio-potential electrode assembly, first short-lead bio-potential input signal(s) in response to electrical activity from a first bio-potential source in the subject's body;
measuring, using the at least one multi-contact bio-potential electrode assembly, first long-lead bio-potential input signal(s) in response to electrical activity from a second bio-potential source in the subject's body;
producing, using a processing unit, a transfer function using the first measured short-lead bio-potential input signal(s) and first measured long lead output signals;
using said transfer function to compute estimated long-lead bio-potential output signal(s) based on second measured short-lead bio-potential input signal(s) from said plurality of contact points; and
subtracting said estimated long-lead bio-potential output signal(s) from second measured long-lead bio-potential output signal(s), thereby identifying residual signal(s) component(s) present in the output signals, said residual signal(s) component(s) substantially representing electrical activity from one or more sources other than the first bio-potential source.

49. The method of claim 48, wherein at least one biopotential short lead(s) and/or long lead(s) of a first electrode assembly provides the calibration biopotential lead(s) output signal(s) to at least another short lead(s) and/or long lead(s) input signal(s) of either the first or a second electrode assembly.

50. The method of claim 48, wherein localization of said residual signal(s) electrical biopotential source within tissue is determined by at least two successive iterations of residual signal(s) computation.

51. The method of claim 48, wherein output bio-potential signal(s) represents mixed maternal-fetal ECG, and input bio-potential signal(s) represents maternal-only ECG, for monitoring heart activity.

52. The method of claim 48, wherein output bio-potential signal(s) represents mixed electroencephalogram (EEG) and electromyogram (EMG), and input biopotential signal(s) represents only EEG, for monitoring brain activity.

53. The method of claim 48, wherein output biopotential signal(s) represents mixed electrocardiogram (ECG) and electroencephalogram (EEG), and input biopotential signal(s) represents only EEG, for monitoring brain activity.

54. The method of claim 48, wherein output biopotential signal(s) represents mixed electrocardiogram (ECG) and electromyogram (EMG), and input biopotential signal(s) represents only ECG, for monitoring heart activity.

55. The method of claim 48, wherein output biopotential signal(s) represents mixed electroencephalogram (EEG) and electrooculogram (EOG), and input biopotential signal(s) represents only EEG, for monitoring brain activity.

56. The method of claim 48, wherein output biopotential signal(s) represents mixed normal electrocardiogram (ECG) and abnormal ECG, and input biopotential signal(s) represents only normal electrocardiogram (ECG), for monitoring heart activity.

57. The method of claim 48, wherein said residual signal(s) represents abnormal cardiac electrocardiogram or arrhythmia of heart activity.

58. The method of claim 48, wherein output biopotential signal(s) represents mixed normal cardiac electrograms (EGM) and abnormal EGM, and input biopotential signal(s) represents only normal EGM, for monitoring heart muscle activity.

59. The method of claim 48, wherein said residual signal(s) represent abnormal cardiac electrogram (EGM) of heart activity.

60. The method of claim 48, wherein output biopotential signal(s) represents mixed normal electroencephalogram (EEG) and abnormal EEG, and input biopotential signal(s) represents only normal EEG, for monitoring brain activity.

61. The method of claim 48, wherein said residual signal(s) represents abnormal brain encephalogram (EEG) of brain activity.

62. The method of claim 48, wherein output biopotential signal(s) represents mixed normal electromyogram (EMG), and abnormal EMG, and input biopotential signal(s) represents only normal EMG, for monitoring muscle activity.

63. The method of claim 48, wherein said residual signal(s) represent abnormal electromyogram (EMG), of muscle activity.

64. The method of claim 48, wherein output biopotential signal(s) represents mixed normal electroneurogram (ENG) and abnormal ENG, and input biopotential signal(s) represents only normal ENG, for monitoring nerve activity.

65. The method of claim 48, wherein said residual signal(s) represent abnormal electroneurogram (ENG) of nerve activity.

66. The method of claim 48, wherein said residual signal(s) represents an evoked potential (EVP) of tissue activity.

67. The method of claim 48, wherein said first short-lead bio-potential input signal(s) and said second short-lead bio-potential input signal(s) are the same; and said first long-lead bio-potential output signal(s) and said second long-lead bio-potential output signal(s) are the same.

68. The method of claim 48, wherein said residual signal(s) from said one or more sources represent stimulated evoked potentials provided at said plurality of contact points for said long-lead biopotential contacts.

69. The method of claim 48, wherein said residual signal(s) from said one or more sources are substantially neutralized by providing an evoked potential representing the inverse of said residual signal(s) at said plurality of contact points for said long-lead biopotential contacts.
